(19) United States
(12) Patent Application Publication
(10) Pub. No.: US 2024/0207442 A1
(43) Pub. Date: Jun. 27, 2024

(54) ALL-IN-ONE DENDRIMER-BASED LIPID NANOPARTICLES ENABLE PRECISE HDR-MEDIATED GENE EDITING IN VIVO

(71) Applicant: The Board of Regents of The University of Texas System, Austin, TX (US)

(72) Inventors: Daniel J. SIEGWART, Dallas, TX (US); Lukas FARBIAK, Dallas, TX (US)

(73) Assignee: The Board of Regents of The University of Texas System, Austin, TX (US)

(21) Appl. No.: 18/556,614

(22) PCT Filed: Apr. 22, 2022
(86) PCT No.: PCT/US2022/026001

§ 371 Date: Oct. 20, 2023

Related U.S. Application Data
(60) Provisional application No. 63/178,453, filed on Apr. 22, 2021.

(51) Int. Cl. A61K 48/00; A61K 9/51; C12N 9/22; C12N 15/10; C12N 15/11; C12N 15/88; C12N 15/90

(52) U.S. Cl. CPC A61K 48/0041; A61K 9/5123; A61K 48/005; C12N 9/22; C12N 15/102; C12N 15/11; C12N 15/88; C12N 15/907; C12N 2310/20

(57) ABSTRACT

In some aspects, the present disclosure provides compositions comprising one or more of each of the following nucleic acids: (1) a mRNA; (2) a sgRNA; and (3) a DNA; and a lipid nanoparticle comprising at least one ionizable lipid; wherein the each of the nucleic acids are encapsulated within the lipid nanoparticle, and pharmaceutical compositions thereof. The present disclosure also provides methods employing said compositions and/or pharmaceutical compositions, such as methods of repairing genes, methods of performing homology directed repair on the genome, and methods of treating diseases or disorders.

### CROSS-REFERENCE

This application claims the benefit of U.S. Provisional Application No. 63/178,453, filed on Apr. 22, 2021, the entire contents of which are hereby incorporated by reference for all purposes.

### STATEMENT REGARDING FEDERALLY SPONSORED RESEARCH OR DEVELOPMENT

This invention was made with government support under Grant No. R01 EB025192-01A1 awarded by the National Institutes of Health (NIH). The government has certain rights in the invention.

### BACKGROUND

### 1. Field

The present invention relates generally to the fields of nucleic acid delivery compositions. For example, in certain aspects, it relates to compositions formulated for delivery of a combination of one or more of the following nucleic acids: mRNA, sgRNA, and DNA; and a lipid nanoparticle comprising at least one ionizable lipid; wherein the each of the nucleic acids are encapsulated within the lipid nanoparticle. For another example, more particularly, it relates to lipid nanoparticle compositions for delivery of such combinations for the treatment of diseases or disorders.

### 2. Description of Related Art

In the short time since its discovery in 2013 (Jinek et al., 2012; Cong et al., 2013; Mali et al., 2013; Doudna and Charpentier, 2014; Sander and Joung, 2014; Wang et al., 2017), CRISPR/Cas (clustered regularly interspaced short palindromic repeat/CRISPR-associated (Cas) protein) has been quickly applied to the treatment of diseases such as Thalassemia, Sickle Cell Disease, Familial Hypercholesterolemia, Duchenne Muscular Dystrophy, cancer, and Leber congenital amaurosis (Long et al., 2016; Min et al., 2019a; Min et al., 2019b; Jo et al., 2019; Wei et al., 2020; Li et al., 2018; Xu et al., 2019; Wang et al., 2019). However, progress to date for in vivo editing has largely been limited to gene knockouts via an error-prone DNA repair mechanism known as Non-Homologous End Joining (NHEJ). True correction of genetic disease and cancer mutations will require Homology Directed Repair (HDR), an approach currently hindered by the lack of carriers that can mediate this complex DNA repair pathway. As such, there exists a significant need for such carriers.

### SUMMARY

In some aspects, the present disclosure provides A composition comprising a lipid composition (e.g., nanoparticle) assembled with a gene or transcript editing composition, wherein:
(A) the gene or transcript editing composition comprises one or more of the following nucleic acids (1)-(3):
   (1) a polynucleotide comprising a sequence encoding a polynucleotide-guided nuclease;
   (2) a guide polynucleotide (e.g., configured to complex with at least a portion of a target gene or transcript, or a polynucleotide comprising a sequence that encodes the guide polynucleotide); and
   (3) a donor polynucleotide (e.g., configured to repair a modified target gene or transcript), and
(B) the lipid composition comprises at least one ionizable lipid.

In some aspects, the present disclosure provides compositions comprising:
(A) one or more of each of the following nucleic acids:
   (1) a polynucleotide comprising a sequence encoding for a polynucleotide-guided nuclease such as an mRNA;
   (2) a guide polynucleotide, particularly a polynucleotide which has been configured to complex with at least a portion of a target gene or transcript or a polynucleotide with a sequence that encodes for such a guide polynucleotide such as a sgRNA; and
   (3) a donor polynucleotide, particularly a polynucleotide configured to repair a modified target gene or transcript such as a DNA;
(B) a lipid nanoparticle comprising at least one ionizable lipid;
wherein the each of the nucleic acids are encapsulated within the lipid nanoparticle.

In some embodiments, the mRNA encodes a gene editing protein. In some embodiments, the mRNA encodes a Cas protein. In some embodiments, mRNA encodes a Cas9 protein. In some embodiments, the DNA is a single stranded DNA. In further embodiments, the single stranded DNA is donor template sequence. In some embodiments, the DNA corrects an error in a genome of a cell. In further embodiments, the composition is capable of correcting an error in the genome of the cell. In some embodiments, the DNA is complementary to a portion of the genome of a cell. In some embodiments, the genome of the cell is the genome of a patient. In some embodiments, the DNA is at least 80% complementary. In further embodiments, the DNA is at least 90% complementary. In still further embodiments, the DNA is at least 95% complementary. In yet further embodiments, the DNA is at least 98% complementary. In further embodiments, the DNA is at least 99% complementary. In some embodiments, the DNA contains a modification relative to the genome of a cell. In some embodiments, the genome of the cell does not encode for the wild type gene. In some embodiments, the DNA encodes for the wild type gene.

In some embodiments, the mRNA comprises from about 250 nucleotides to about 15,000 nucleotides. In further embodiments, the mRNA comprises from about 500 nucleotides to about 5,000 nucleotides. In still further embodiments, the mRNA comprises from about 800 nucleotides to about 2,500 nucleotides. In some embodiments, the sgRNA comprises from about 25 nucleotides to about 500 nucleotides. In further embodiments, the sgRNA comprises from about 50 nucleotides to about 300 nucleotides. In still further embodiments, the sgRNA comprises from about 80 nucleotides to about 200 nucleotides.

In some embodiments, the DNA comprises from about 25 nucleotides to about 2,500 nucleotides. In some embodiments, the DNA comprises from about 25 nucleotides to about 200 nucleotides. In some embodiments, the DNA comprises from about 50 nucleotides to about 300 nucleotides. In some embodiments, the DNA comprises from about 80 nucleotides to about 200 nucleotides.

In some embodiments, the composition comprises a weight ratio of mRNA to sgRNA from about 10:1 to about 1:5. In further embodiments, the weight ratio of mRNA to sgRNA is from about 5:1 to about 1:3. In still further embodiments, the weight ratio of mRNA to sgRNA is from about 3:1 to about 1:2. In yet further embodiments, the weight ratio of mRNA to sgRNA is 2:1, 1:1, or 1:2. In some embodiments, the composition comprises a weight ratio of mRNA to DNA from about 2:1 to about 1:20. In further embodiments, the weight ratio of mRNA to DNA is from about 1:1 to about 1:10. In still further embodiments, the weight ratio of mRNA to DNA is from about 1:2 to about 1:8. In yet further embodiments, the weight ratio of mRNA to DNA is 1:3 or 1:4. In some embodiments, the composition comprises a weight ratio of sgRNA to DNA from about 4:1 to about 1:10. In further embodiments, the weight ratio of sgRNA to DNA is from about 2:1 to about 1:8. In still further embodiments, the weight ratio of sgRNA to DNA is from about 1:1 to about 1:4. In yet further embodiments, the weight ratio of sgRNA to DNA is 2:3 or 1:2.

In some embodiments, the ionizable lipid is a cationic lipid. In some embodiments, the ionizable lipid is a dendron or dendrimer. In some embodiments, the ionizable lipid is a compound of the formula:

Core-Repeating Unit-Terminating Group (D-I)

wherein the core is linked to the repeating unit by removing one or more hydrogen atoms from the core and replacing the atom with the repeating unit and wherein:
the core has the formula:

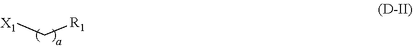

wherein:
X₁ is amino or alkylamino_{(C≤12)}, dialkylamino_{(C≤12)}, heterocycloalkyl_{(C≤12)}, heteroaryl_{(C≤12)}, or a substituted version thereof;
R₁ is amino, hydroxy, or mercapto, or alkylamino_{(C≤12)}, dialkylamino_{(C≤12)}, or a substituted version of either of these groups; and
a is 1, 2, 3, 4, 5, or 6; or
   the core has the formula:

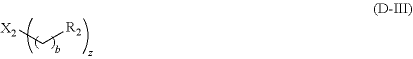

wherein:
X₂ is N(R₅)_{y};
R₅ is hydrogen, alkyl_{(C≤18)}, or substituted alkyl_{(C≤18)}; and
y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3; or
   the core has the formula:

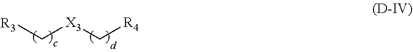

wherein:
X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

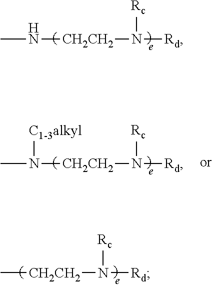

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
   c and d are each independently 1, 2, 3, 4, 5, or 6; or
   the core is alkylamine_{(C≤18)}, dialkylamine_{(C≤36)}, heterocycloalkane_{(C≤12)}, or a substituted version of any of these groups;
      wherein the repeating unit comprises a degradable diacyl and a linker;
      the degradable diacyl group has the formula:

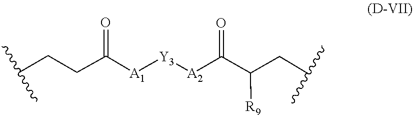

wherein:
A₁ and A₂ are each independently —O—, —S—, or —NRₐ—, wherein:
Rₐ is hydrogen, alkyl_{(C≤6)}, or substituted alkyl_{(C≤6)};
Y₃ is alkanediyl_{(C≤12)}, alkenediyl_{(C≤12)}, arenediyl_{(C≤12)}, or a substituted version of any of these groups; or a group of the formula:

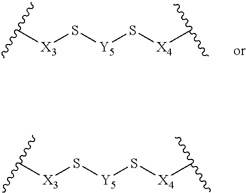

wherein:
   X₃ and X₄ are alkanediyl_{(C≤12)}, alkenediyl_{(C≤12)}, arenediyl_{(C≤12)}, or a substituted version of any of these groups;
   Y₅ is a covalent bond, alkanediyl_{(C≤12)}, alkenediyl_{(C≤12)}, arenediyl_{(C≤12)}, or a substituted version of any of these groups; and
   R₉ is alkyl_{(C≤8)} or substituted alkyl_{(C≤8)};
      the linker group has the formula:

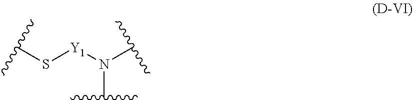

wherein:
Y₁ is alkanediyl_{(C≤12)}, alkenediyl_{(C≤12)}, arenediyl_{(C≤12)}, or a substituted version of any of these groups; and
   wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
   the terminating group has the formula:

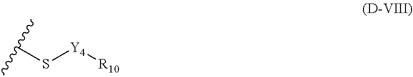

wherein:
Y₄ is alkanediyl_{(C≤24)}, alkanediyl_{(C≤24)}, or a substituted version thereof;
R₁₀ is hydrogen, amino, carboxy, hydroxy, or aryl_{(C≤12)}, alkylamino_{(C≤12)}, dialkylamino_{(C≤12)}, N-heterocycloalkyl_{(C≤12)}, —C(O)N(R₁₁)-alkanediyl_{(C≤6)}-heterocycloalkyl_{(C≤12)}, —C(O)-alkylamino_{(C≤12)}, —C(O)-dialkylamino_{(C≤12)}, —C(O)—N-heterocycloalkyl_{(C≤12)}, wherein:
R₁₁ is hydrogen, alkyl_{(C≤6)}, or substituted alkyl_{(C≤6)};
wherein the final degradable diacyl in the chain is attached to a terminating group;
n is 0, 1, 2, 3, 4, 5, or 6;
   or a pharmaceutically acceptable salt thereof.

In some embodiments, the core is further defined by the formula:

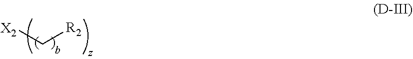

wherein:
X₂ is N(R₅)_{y};
   R₅ is hydrogen or alkyl_{(C≤8)}, or substituted alkyl_{(C≤18)}; and
y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3.

In some embodiments, the core is further defined as:

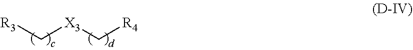

wherein:
X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

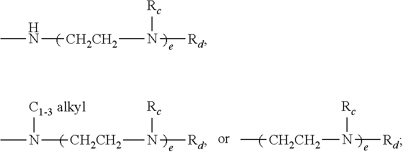

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
c and d are each independently 1, 2, 3, 4, 5, or 6.

In some embodiments, the core is further defined as:

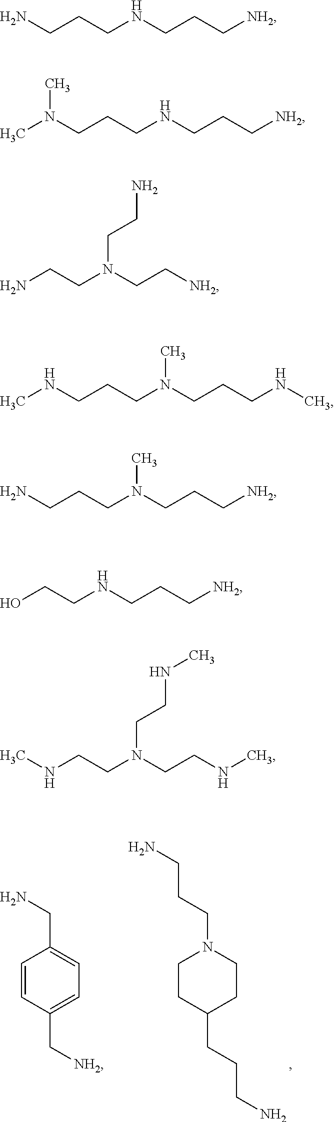

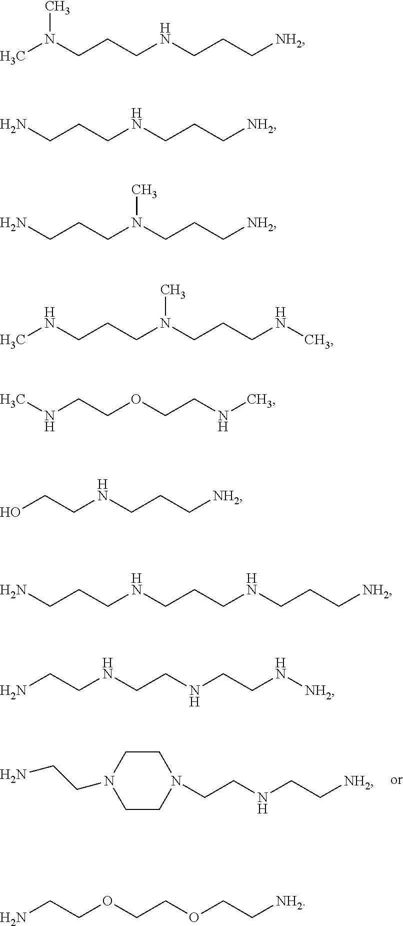

In some embodiments, the core is further defined as:

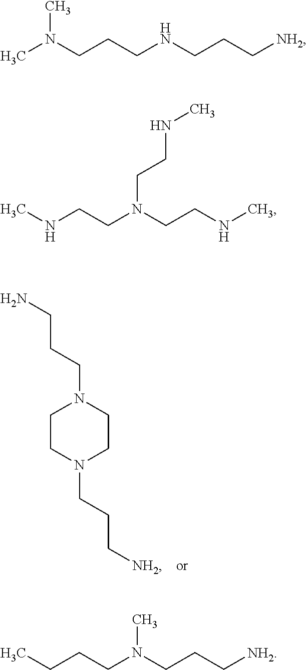

In some embodiments, the core is further defined as:

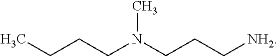

In some embodiments, A₁ and A₂ are O. In some embodiments, Y₃ is alkanediyl_{(C≤12)} or substituted alkanediyl_{(C≤12)}. In some embodiments, Y₁ is alkanediyl_{(C≤12)} or substituted alkanediyl_{(C≤12)}.

In some embodiments, the terminating group is further defined as:

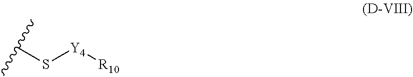

wherein:
Y₄ is alkanediyl_{(C≤18)} or alkenediyl_{(C≤18)}; and
R₁₀ is hydrogen.

In some embodiments, the terminating group is further defined as:

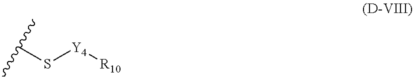

wherein:
Y₄ is alkanediyl_{(C≤18)}; and
R₁₀ is hydrogen.

In some embodiments, the dendrimer or dendron is further defined as:

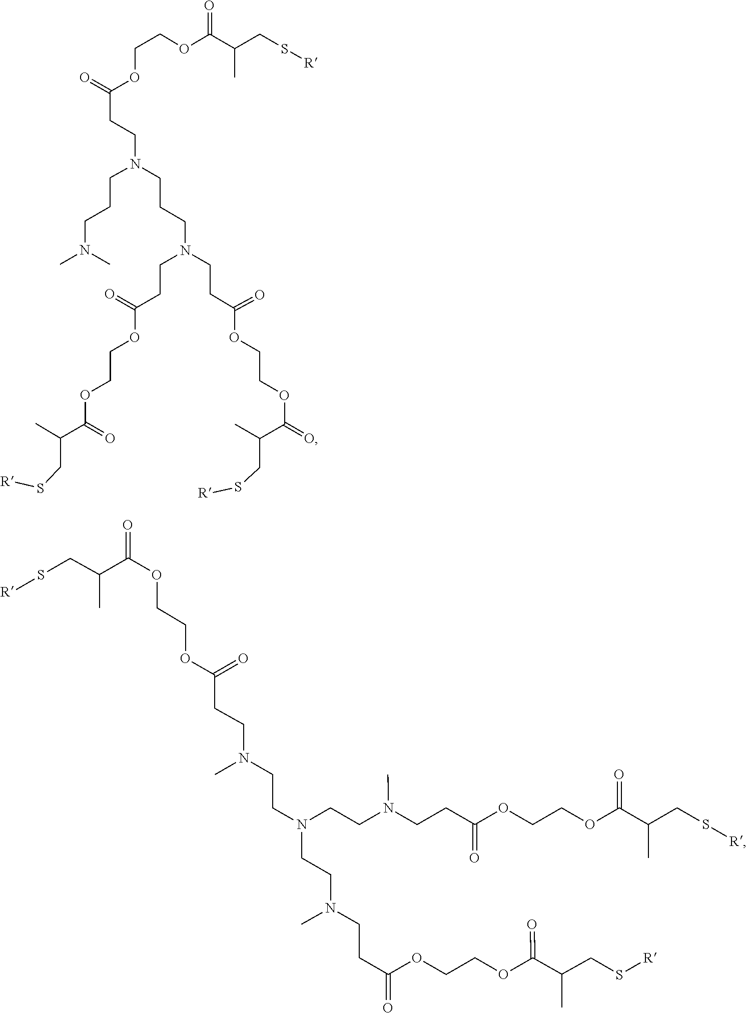

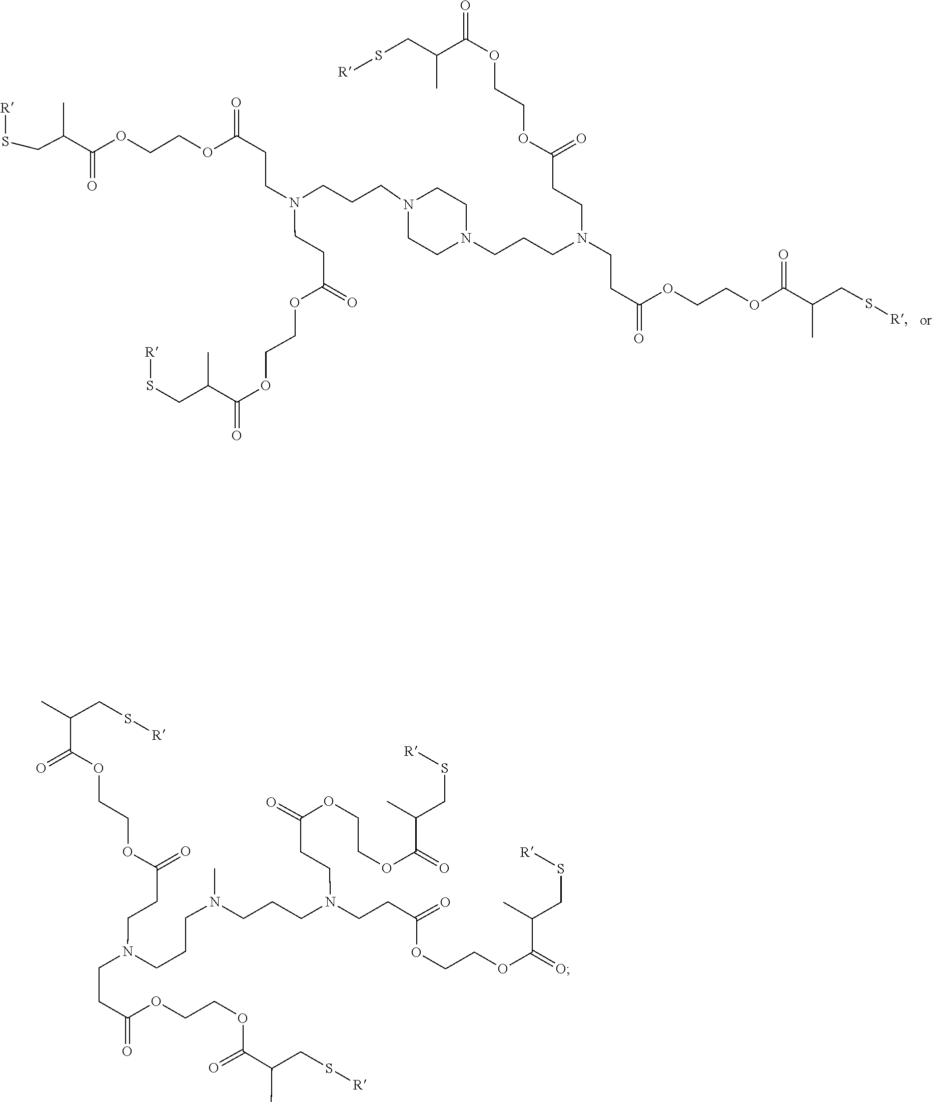

wherein:
R′ is alkyl_{(C≤18)}, alkenyl_{(C≤18)}, or a substituted version thereof.

In some embodiments, the dendrimer or dendron is further defined as:

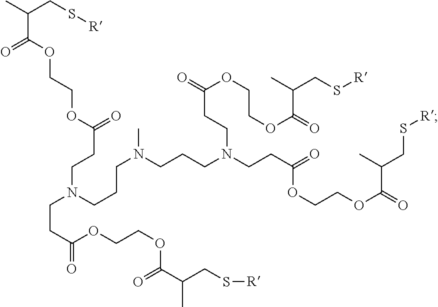

wherein:
R′ is alkyl_{(C≤18)}, alkenyl_{(C≤18)}, or a substituted version thereof.

In some embodiments, the dendrimer or dendron is further defined as:

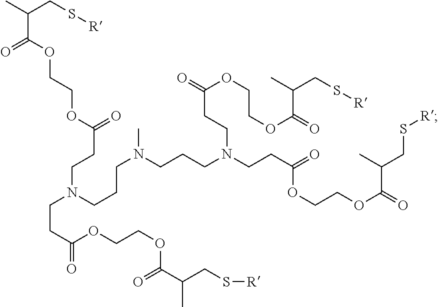

wherein:
R′ is alkyl_{(C6-18)}.

In some embodiments, the composition comprises a molar ratio from about 15 to about 60 of the ionizable lipid relative to any other lipids. In further embodiments, the molar ratio is from about 25 to about 50 of the ionizable lipid. In still further embodiments, the molar ratio is from about 30 to about 45 of the ionizable lipid.

In some embodiments, the composition further comprises a phospholipid. In further embodiments, the phospholipid comprises one or two long chain alkyl or alkenyl groups, a glycerol or a sphingosine, one or two phosphate groups, and a small organic molecule, wherein the small organic molecule is an amino acid, a sugar, or an amino substituted alkoxy group. In still further embodiments, the phospholipid is 1,2-distearoyl-sn-glycero-3-phosphocholine (DSPC) or 1,2-dioleoyl-sn-glycero-3-phosphoethanolamine (DOPE). In some embodiments, the phospholipid is DOPE. In some embodiments, the composition comprises a molar ratio from about 5 to about 50 of the phospholipid relative to any other lipids. In further embodiments, the molar ratio is from about 10 to about 45 of the phospholipid. In still further embodiments, the molar ratio is from about 20 to about 40 of the phospholipid. In some embodiments, the composition further comprises a steroid, such as cholesterol. In some embodiments, the composition comprises a molar ratio from about 10 to about 60 of the steroid relative to any other lipids. In further embodiments, the molar ratio is from about 15 to about 50 of the steroid. In still further embodiments, the molar ratio is from about 25 to about 50 of the steroid.

In some embodiments, the composition further comprises a PEGylated lipid. In further embodiments, the PEGylated lipid comprises a PEG component from about 1000 to about 10,000 daltons. In some embodiments, the PEG lipid is a PEGylated diacylglycerol. In some embodiments, the PEG lipid is further defined by the formula:

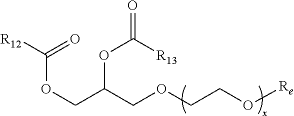

wherein:
R₁₂ and R₁₃ are each independently alkyl_{(C≤24)}, alkenyl_{(C≤24)}, or a substituted version of either of these groups;
Rₑ is hydrogen, alkyl_{(C≤8)}, or substituted alkyl_{(C≤8)}; and
x is 1-250.

In some embodiments, the PEG lipid is dimyristoyl-sn-glycerol or a compound of the formula:

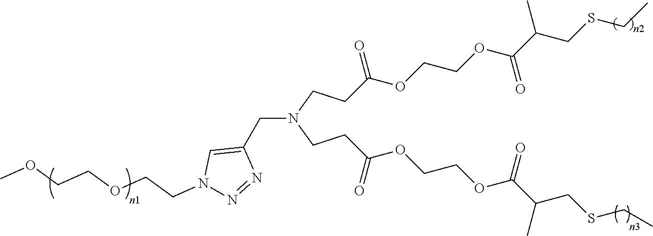

wherein:
n₁ is 5-250; and
n₂ and n₃ are each independently 2-25.

In some embodiments, the composition comprises a molar ratio from about 0.25 to about 12.5 of the PEGylated lipid relative to any other lipids. In further embodiments, the molar ratio is from about 0.5 to about 10 of the PEGylated lipid. In still further embodiments, the molar ratio is from about 1 to about 6 of the PEGylated lipid.

In some embodiments, the composition comprises a molar ratio of lipid components to nucleic acid components of from about 1,000:1 to about 5,000:1. In further embodiments, the composition comprises a molar ratio of lipid components to nucleic acid components of from about 2,000:1 to about 4,000:1. In still further embodiments, the composition comprises a molar ratio of lipid components to nucleic acid components of about 2,500:1. In some embodiments, the composition comprises 4AC3-SC8, cholesterol, DOPE, and DMG-PEG2000. In some embodiments, the composition comprises a molar ratio of 4AC3-SC8:cholesterol:DOPE:DMG-PEG2000 of from about 38.5:30:30:1.5.

In some embodiments, the composition comprises an N:P ratio of from about 1:1 to about 20:1. In further embodiments, the N:P ratio is from about 2:1 to about 10:1. In still further embodiments, the N:P ratio is from about 4:1 to about 8:1. In some embodiments, the composition results in a homology directed repair rate of at least 1%. In further embodiments, the composition results in a homology directed repair rate of at least 5%. In still further embodiments, the composition results in a homology directed repair rate of at least 15%. In yet further embodiments, the homology directed repair rate is at least 25%. In further embodiments, the homology directed repair rate is at least 50%. In some embodiments, the composition has an indel rate of less than 5%. In further embodiments, the indel rate is less than 2%. In still further embodiments, the indel rate is less than 1%.

In other aspects, the present disclosure provides compositions for use in homology directed repair comprising:
(A) one or more of each of the following nucleic acids:
   (1) a polynucleotide comprising a sequence encoding for a polynucleotide-guided nuclease such as an mRNA;
   (2) a guide polynucleotide, particularly a polynucleotide which has been configured to complex with at least a portion of a target gene or transcript or a polynucleotide with a sequence that encodes for such a guide polynucleotide such as a sgRNA; and
   (3) a donor polynucleotide, particularly a polynucleotide configured to repair a modified target gene or transcript such as a DNA;
(B) a lipid nanoparticle comprising at least one ionizable lipid.

In still other aspects, the present disclosure provides pharmaceutical compositions comprising:
(A) a composition of the present disclosure; and
(B) a pharmaceutically acceptable carrier.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In further embodiments, the pharmaceutical composition is formulated for injection. In some embodiments, the pharmaceutical composition is formulated as a unit dose.

In yet other aspects, the present disclosure provides methods of repairing a gene comprising administering to a patient in need thereof a composition of the present disclosure. In some embodiments, the DNA encodes for the wild type allele of the gene to be repaired.

In other aspects, the present disclosure provides methods of performing homology directed repair on the genome of a cell comprising administering to a cell a composition of the present disclosure. In some embodiments, the cell is a plurality of cells. In further embodiments, the plurality of cells is a patient.

In still other aspects, the present disclosure provides methods of treating a disease or disorder in a patient comprising administering to the patient a therapeutically effective amount of a composition of the present disclosure. In some embodiments, the disease or disorder is a genetic disease or disorder. In some embodiments, the disease or disorder is associated with a mutation in one or more genes. In some embodiments, the method further comprises administering a second therapy to the patient. In some embodiments, the method further comprises administering the composition to the patient once. In some embodiments, the method further comprises administering the composition to the patient two or more times.

The terms “comprise” (and any form of comprise, such as “comprises” and “comprising”), “have” (and any form of have, such as “has” and “having”), “contain” (and any form of contain, such as “contains” and “containing”), and “include” (and any form of include, such as “includes” and “including”) are open-ended linking verbs. As a result, a method, composition, kit, or system that “comprises,” “has,” “contains,” or “includes” one or more recited steps or elements possesses those recited steps or elements, but is not limited to possessing only those steps or elements; it may possess (i.e., cover) elements or steps that are not recited. Likewise, an element of a method, composition, kit, or system that “comprises,” “has,” “contains,” or “includes” one or more recited features possesses those features, but is not limited to possessing only those features; it may possess features that are not recited.

Any embodiment of any of the present methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 shows 4A3-SC8 HDR dLNPs effectively orchestrate HDR-mediated gene editing in vitro and in vivo. All-in-one dLNPs containing three nucleic acids (Cas9 mRNA, sgRNA, and ssDNA) are able to escape the endosome and release nucleic acid cargoes into the cytoplasm. Cas9 mRNAs are then translated into Cas9 proteins which associate with sgRNAs to form ribonucleoprotein complexes (RNPs). RNPs/ssDNA template traverse the nuclear membrane and locate their target site in the genomic DNA creating a double-stranded break (DSB) wherein the ssDNA template containing a corrected sequence is incorporated into the genomic DNA.

FIG. 2 shows the internal molar ratios of 4A3-SC8, Cholesterol, DOPE, and PEG-DMG were varied in a systematic fashion to create particles with different lipid properties. Various formulations were screened where 50 ng of luciferase mRNA was delivered per well. Luciferase activity and cytotoxicity were measured (N=4+/−standard deviation). See Table 1 for formulation details.

FIGS. 3A-3F shows the chemical identity of dendrimers in dLNPs influences luciferase mRNA delivery efficacy across cell types. (FIG. 3A) Modular degradable dendrimers were synthesized containing an ionizable amine core, ester linkages, and alkyl-thiol peripheries via sequential Michael addition. (FIG. 3B) Four different amine cores and nine alkyl peripheries were selected to form a dendrimer library consisting of 36 distinct structures for efficacy assessment. (FIG. 3C) HEK293T, HeLa, and IGROV1 cells were transfected with dLNPs containing firefly luciferase mRNA at a dose of 12.5 ng (6.672 mM) and analyzed for fold increase in bioluminescence after normalization to background and viability (N=4). (FIG. 3D) All cells were assessed for viability following transfection with each of the dLNP formulations and exhibited minimal to no cytotoxicity (N=4). (FIG. 3E) Most dLNPs were approximately 100 nm in diameter and uniform (PDI<0.2; N=5). (FIG. 3F) mRNA encapsulation did not vary significantly between dLNP formulations (N=4).

FIG. 4 shows a dose response experiment was performed with dLNPs containing luciferase mRNA. The dLNPs were comprised of the 4A3 amine core and 9 different alkyl peripheries (SC5, SC6, SC7, SC8, SC9, SC10, SC11, SC12, SC14) in HeLa Cells. Notably, HeLa cells exhibited dramatic increases in luminescence in accordance with increasing dose of luciferase mRNA (N=4).

FIG. 5 shows a dose response experiment was performed with dLNPs containing luciferase mRNA. The dLNPs were comprised of the 4A3 amine core and 9 different alkyl peripheries (SC5, SC6, SC7, SC8, SC9, SC10, SC11, SC12, SC14) in IGROV1 cells (N=4).

FIG. 6 shows a dose response experiment was performed with dLNPs containing luciferase mRNA. The dLNPs were comprised of the 4A3 amine core and 9 different alkyl peripheries (SC5, SC6, SC7, SC8, SC9, SC10, SC11, SC12, SC14) in HEK293T cells (N=4).

FIG. 7 shows the ONE-Glo+Tox assay was used to assess cell viability in the dose response experiment performed with dLNPs containing luciferase mRNA. The dLNPs were comprised of the 4A3 amine core and 9 different alkyl peripheries (SC5, SC6, SC7, SC8, SC9, SC10, SC11, SC12, SC14). (N=4).

FIG. 8 shows quantitation of editing via NHEJ and HDR was performed via flow cytometry. In the above representative figures, HEK293 B/GFP cells with no fluorescence are shown as a negative control for gating purposes and quantification of editing via NHEJ wherein cells lose fluorescence due to the introduction of indels. Additionally, unedited cells are shown wherein blue fluorescence is observed. Finally, cells that have undergone gene correction via HDR are shown as indicated by a shift in the population of cells upwards on the GFP axis, indicating bright green fluorescence.

FIGS. 9A-9F show 4A3-SC8 dLNPs successfully induced HDR in HEK293 cells containing a GFP sequencing with a Y66H mutation via one-pot delivery of Cas9 mRNA, sgRNA, and a corrected ssDNA template. (FIG. 9A) HEK293 cells contain a single amino acid mutation in their GFP sequence (Y66H) that alters their fluorescence. Depending on the gene editing technique employed, the fluorescence can be eliminated (NHEJ) or restored to native GFP (HDR). (FIG. 9B) HEK293 (Y66H) cells were transfected with dLNPs containing 1000 ng of only Cas9 mRNA and sgRNA at a 2:1 ratio or dLNPs containing Cas9 mRNA, sgRNA, and ssDNA template at a 2:1:1 ratio and imaged using confocal microscopy for mutated GFP (blue) and GFP (green) signal. CellMask Orange was used to stain plasma membranes in merged images. The amount and type of gene editing induced were assessed using TIDER to analyze DNA sequencing following transfection with HDR dLNPs containing Cas9 mRNA:sgRNA ratios fixed at (FIG. 9C) 1:1, (FIG. 9D) 1:2, and (FIG. 9E) 2:1 (N=3). (FIG. 9F) Across all three fixed ratios of Cas9 mRNA:sgRNA similar trends in the amount of HDR, NHEJ, total efficacy, and unedited cells were observed, with the 2:1 Cas9 mRNA:sgRNA exhibiting the highest degree of all forms of editing (N=3).

FIG. 10 shows sequential delivery was achieved via a two-particle system wherein the first dLNPs were loaded only with Cas9 mRNA and administered to the cells at a dose of 500 ng. 24 h after transfection with the Cas9 mRNA dLNPs, a second round of particles containing either sgRNA only or sgRNA and ssDNA template were administered to the cells at doses of 250 ng for sgRNA only particles and 500 ng total nucleic acid for the particles containing sgRNA and ssDNA template.

FIG. 11 shows simultaneous delivery was achieved via multi or single particle systems. The first dLNP system consisted of three different particles each loaded only with one nucleic acid, either Cas9 mRNA 500 ng, sgRNA 250 ng, or ssDNA template 250 ng. All particles were administered to the cells at the same time point. The second system used two particles, one loaded with Cas9 mRNA (500 ng) and the other loaded with sgRNA and ssDNA template (500 ng total nucleic acid) which were administered to the cells at the same time point. Finally, a particle containing 1000 ng total nucleic acid at a 2:1:1 ratio of Cas9 mRNA:sgRNA:ssDNA template was administered to the cells.

FIG. 12 shows three 4A3-SC8 dLNPs were created using the same lipid composition and loaded with 250 ng sgRNA only, 250 ng sgRNA and 500 ng Cas9 mRNA, or 250 ng sgRNA, 500 ng Cas9 mRNA, and 750 ng ssDNA. The Ribogreen assay was used to evaluate nucleic acid encapsulation in each of the three 4A3-SC8 dLNPs revealing that the amount of encapsulated nucleic acid increased in each formulation. Encapsulation efficiency was >75% for all three dLNPs

FIG. 13 shows HDR dLNPs were characterized for size and polydispersity using dynamic light scattering.

FIG. 14 shows HDR dLNPs were created containing differing ratios of nucleic acid components wherein the ratio of Cas9 mRNA:sgRNA was fixed at one of three ratios (1:1, 1:2, or 2:1) by weight (1=250 ng) and the amount of ssDNA HDR template was titrated in at ratios of 0.5, 1, 2, 3, 4, 6, 8, or 10. Following treatment with HDR dLNPs, the cells were analyzed for GFP+ (HDR), BFP+ (unedited) or non-fluorescent (NHEJ) signal using flow cytometry. HDR was accomplished in all groups, however, the amount of HDR, NHEJ, and unedited cells changed with respect to ratio of nucleic acid components in each of the dLNP formulations (N=3).

FIGS. 15A-15E show HDR gene correction was achieved in vivo. (FIG. 15A) Subcutaneous xenograft HEK293 (Y66H) tumors were resected 5 days after injection of 4A3-SC8 HDR dLNPs for analysis by IVIS Lumina imaging, flow cytometry, confocal microscopy, and DNA sequencing. (FIG. 15B) IVIS Lumina imaging of tumors and organs revealed bright GFP signal in the tumors injected with 1:1:8, 1:1:3, and 2:1:3 HDR dLNPs and no detectable GFP signal in the tumors injected with PBS (N=3). (FIG. 15C) Average radiance of tumors in each HDR dLNP group was quantified revealing a significant difference between all three HDR dLNP and PBS injected tumors (for 1:1:8 p=0.0060, for 1:1:3 p=0.0106, for 2:1:3 p=0.0008; statistical analysis was performed using one-way ANOVA with Tukey's multiple comparisons test; N=3). (FIG. 15D) Tumors were frozen following resection and sectioned at a thickness of 7 mm for confocal imaging wherein GFP signal was clearly visible in tumors injected with HDR dLNPs. (FIG. 15E) gDNA was extracted from whole tumors and then sequenced to obtain HDR correction percentage. Analysis via TIDER revealed average HDR-mediated correction rates of 10.475% for 1:1:8 HDR dLNPs, 16.533% for 1:1:3 HDR dLNPs, and 20.325% for 2:1:3 HDR dLNPs (Statistical analysis was performed using one-way ANOVA with Tukey's multiple comparisons test, N=4 with exception of 1:1:3 where N=3).

FIG. 16 shows the sgRNA sequence was evaluated for target site cutting using an in vitro cutting assay. The sgRNA used in this assay was created using IVT. Cutting at the target site is indicated in the agarose gel image above by the presence of multiple bands in the group that was incubated with sgRNA and Cas9 protein.

FIG. 17 shows modified sgRNA was encapsulated along with Cas9 mRNA into dLNPs at a ratio of 2:1 by weight (500 ng Cas9 mRNA, 250 ng modified sgRNA). Similarly, 250 ng of modified sgRNA was encapsulated into dLNPs that did not contain Cas9 mRNA. The dLNPs were administered to HEK293 B/GFP cells and the cells were incubated at 37° C. for 48 h before being collected and prepared for analysis via flow cytometry. In the group receiving dLNPs containing both Cas9 mRNA and modified sgRNA there was significant gene editing via NHEJ as indicated by the large amount of non-fluorescent cells (white bar, 58%). Editing was further confirmed by controlling for antisense activity that is known to be induced by the presence of sgRNA in cells. As indicated above, when modified sgRNA was delivered to cells alone, the amount of non-fluorescent cells present was similar to the PBS control.

FIG. 18 shows nude mice bearing HEK293 B/GFP tumors were injected intravenously (IV) or intratumorally (IT) with 4A3-SC8 dLNPs containing luciferase mRNA at a dose of 0.25 mg/kg. 6 h post IT/IV injection, mice were administered d-luciferin and imaged using IVIS for luminescence. In mice injected IT with the Luc mRNA 4A3-SC8 dLNPs, imaging at 6 h showed bright luminescence in the tumor mass and no luminescence in other tissues. In mice injected IV with Luc mRNA 4A3-SC8 dLNPs, bright luminescence can be seen in the liver, but no luminescence is visible in the tumor mass. To ensure enough time passed for any potential leakage of dLNPs into systemic circulation, mice were imaged 24 h following the initial IV/IT injection of dLNPs. Again, luminescence was present only in the tumor mass of mice injected IT. In mice injected IV, luminescence was only present in the liver. Additionally, the tumor mass, heart, lungs, liver, kidneys, and spleen were resected from the mice and imaged for luminescence. Confirming previous images, bright luminescence was visible only in the tumor mass of mice injected IT, indicating no leak through of dLNPs into systemic circulation. Mice injected IV with dLNPs showed bright luminescence in the liver and minor luminescence in spleen, but no luminescence in tumor mass.

FIG. 19 shows Cas-OFFinder was used to identify 5 top off-target sites for PTEN in the mouse genome. Nude mice bearing HEK293 B/GFP tumors were injected intratumorally with a 2:1 ratio of Cas9 mRNA:PTEN sgRNA at a dose of 0.25 mg/kg and analyzed for on-target and off-target editing via Sanger DNA Sequencing and the T7E1 assay.

FIG. 20 shows Sanger DNA sequencing and the T7E1 assay revealed no on-target or off-target editing at any of the 5 predicted off-target sites in liver following IT injection with 4A3-SC8 dLNPs containing a 2:1 ratio of Cas9 mRNA:PTEN sgRNA at a dose of 0.25 mg/kg.

FIG. 21 shows Sanger DNA sequencing and the T7E1 assay revealed no on-target or off-target editing at any of the 5 predicted off-target sites in lung following IT injection with 4A3-SC8 dLNPs containing a 2:1 ratio of Cas9 mRNA:PTEN sgRNA at a dose of 0.25 mg/kg.

FIG. 22 shows Sanger DNA sequencing and the T7E1 assay revealed no on-target or off-target editing at any of the 5 predicted off-target sites in spleen following IT injection with 4A3-SC8 dLNPs containing a 2:1 ratio of Cas9 mRNA:PTEN sgRNA at a dose of 0.25 mg/kg.

FIG. 23 shows the Cas-OFFinder webtool was used to predict likely off-target editing sites for the B/GFP sgRNA. Five top potential off-target sites were amplified by PCR following transfection of HEK293 B/GFP cells with 4A3-SC8 dLNPs containing a 2:1 ratio of Cas9mRNA:sgRNA and then analyzed using the T7E1 assay. On-target amplicons showed clear cleavage bands of the correct predicted size but no editing at any of the five off-target sites.

FIG. 24 shows the Cas-OFFinder webtool was used to predict likely off-target editing sites for the B/GFP sgRNA. Five top potential off-target sites were amplified by PCR following transfection of HEK293 B/GFP cells with 4A3-SC8 dLNPs containing a 2:1 ratio of Cas9mRNA:sgRNA and then analyzed using the T7E1 assay and Sanger sequencing. Sequencing data revealed clear on-target editing with small, undefined peaks around the target cleavage site. In the off-target samples, sharp, clear peaks were present around the potential cleavage sites, indicating no Indel formation.

FIG. 25 shows flow cytometry was used to asses HDR efficiency of 1:1:3 4A3-SC8 HDR dLNPs in comparison to the commercially available reagents Lipofectamine 2000 and RNAiMAX which were both loaded with a 1:1:3 ratio of Cas9mRNA:sgRNA:ssDNA and administered to HEK293 B/GFP cells. Analysis for GFP+ cells revealed little HDR induction for the Lipofectamine 2000 (<5%) and RNAiMAX (<2%) transfection reagents in comparison to the 4A3-SC8 HDR dLNPs (>35%). Additionally, HEK293 B/GFP cells were transfected with 4A3-SC8 dLNPs containing scrambled sgRNA in place of B/GFP sgRNA (SCsgRNA), scrambled DNA template in place of ssDNA B/GFP template (SCssDNA), Cas9 mRNA and ssDNA but no sgRNA, and sgRNA and Cas9 mRNA but no ssDNA. In all cases other than the dLNP containing no ssDNA template HDR induction was <1%.

FIG. 26 shows flow cytometry was used to asses cytotoxicity after treatment with 4A3-SC8 dLNPs containing a 1:1:3 ratio of Cas9 mRNA:sgRNA:ssDNA (1000 ng of total nucleic acids) as well as Lipofectamine 2000 and RNAiMAX containing the same dose and ratio of nucleic acids. HEK293 B/GFP cells were plated in 500 uL of DMEM (10% FBS, 1% penicillin/streptomycin) in a 12 well plate at a density of 1.5×10⁵ cells/well and allowed to attach overnight at 37° C. prior to transfection. 24 h following transfection, an additional 1 mL of DMEM was added to each well. The cells were prepared for analysis via flow cytometry 48 h after initial transfection. No significant toxicity was observed as compared with PBS in cells treated with the 1:1:3 4A3-SC8 HDR dLNPs or RNAiMAX. However, ˜12% toxicity was present in cells treated with Lipofectamine 2000 (N=3+/−standard deviation; statistical analysis performed using one-way ANOVA with Dunnett's multiple comparisons test against PBS).

### DESCRIPTION OF ILLUSTRATIVE EMBODIMENTS

In some aspects, the present disclosure provides lipid nanoparticle compositions for use in the delivery of one or more of each of the following nucleic acids: (1) a mRNA; (2) a sgRNA; and (3) a DNA; and a lipid nanoparticle comprising at least one ionizable lipid; wherein the each of the nucleic acids are encapsulated within the lipid nanoparticle. These compositions may be used to treat diseases and disorders for which an mRNA, sgRNA, and DNA would be useful, such as diseases or disorders associated with a mutation in one or more genes.

### A. CRISPR Systems

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor (e.g., KRAB) or activator, to affect gene expression. Alternatively, a CRISPR system with a catalytically inactivate Cas9 further comprises a transcriptional repressor or activator fused to a ribosomal binding protein.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as a DNA template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

The elements of the CRISPR system can be introduced into a cell such that expression of the elements of the CRISPR system direct formation of the CRISPR complex at one or more target sites. Components can be delivered to cells as proteins and/or RNA. For example, a Cas enzyme can be delivered as an mRNA encoding the Cas enzyme, the guide RNA can be delivered as an sgRNA, and the DNA template for HDR can be delivered as a DNA.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce HDR.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### B. Ionizable Lipids

In some aspects of the present disclosure, composition containing compounds containing lipophilic and cationic components, wherein the cationic component is ionizable, are provided. In some embodiments, these cationic ionizable lipids are dendrimers, which are a polymer exhibiting regular dendritic branching, formed by the sequential or generational addition of branched layers to or from a core and are characterized by a core, at least one interior branched layer, and a surface branched layer. (See Petar R. Dvornic and Donald A. Tomalia in Chem. in Britain, 641-645, August 1994.) In other embodiments, the term “dendrimer” as used herein is intended to include, but is not limited to, a molecular architecture with an interior core, interior layers (or “generations”) of repeating units regularly attached to this initiator core, and an exterior surface of terminal groups attached to the outermost generation. A “dendron” is a species of dendrimer having branches emanating from a focal point which is or can be joined to a core, either directly or through a linking moiety to form a larger dendrimer. In some embodiments, the dendrimer structures have radiating repeating groups from a central core which doubles with each repeating unit for each branch. In some embodiments, the dendrimers described herein may be described as a small molecule, medium-sized molecules, lipids, or lipid-like material. These terms may be used to described compounds described herein which have a dendron like appearance (e.g., molecules which radiate from a single focal point).

While dendrimers are polymers, dendrimers may be preferable to traditional polymers because they have a controllable structure, a single molecular weight, numerous and controllable surface functionalities, and traditionally adopt a globular conformation after reaching a specific generation. Dendrimers can be prepared by sequentially reactions of each repeating unit to produce monodisperse, tree-like and/or generational structure polymeric structures. Individual dendrimers consist of a central core molecule, with a dendritic wedge attached to one or more functional sites on that central core. The dendrimeric surface layer can have a variety of functional groups disposed thereon including anionic, cationic, hydrophilic, or lipophilic groups, according to the assembly monomers used during the preparation. In some embodiments, the ionizable cationic lipid is a dendrimer or dendron further defined by the formula:

Core-Repeating Unit-Terminating Group (D-I)

wherein the core is linked to the repeating unit by removing one or more hydrogen atoms from the core and replacing the atom with the repeating unit and wherein:
the core has the formula:

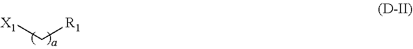

wherein:
   X₁ is amino or alkylamino_{(C≤12)}, dialkylamino_{(C≤12)}, heterocycloalkyl_{(C≤12)}, heteroaryl_{(C≤12)}, or a substituted version thereof;
   R₁ is amino, hydroxy, or mercapto, or alkylamino_{(C≤12)}, dialkylamino_{(C≤12)}, or a substituted version of either of these groups; and
   a is 1, 2, 3, 4, 5, or 6; or
the core has the formula:

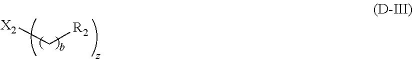

wherein:
   X₂ is N(R₅)_{y};
   R₅ is hydrogen, alkyl_{(C≤18)}, or substituted alkyl_{(C≤18)}; and
   y is 0, 1, or 2, provided that the sum of y and z is 3;
   R₂ is amino, hydroxy, or mercapto, or alkylamino_{(C≤12)}, dialkylamino_{(C≤12)}, or a substituted version of either of these groups;
   b is 1, 2, 3, 4, 5, or 6; and
   z is 1, 2, 3; provided that the sum of z and y is 3; or
the core has the formula:

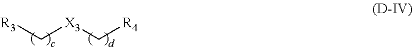

wherein:
   X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c})ₑR_{d},

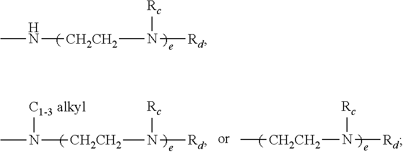

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
c and d are each independently 1, 2, 3, 4, 5, or 6; or
the core is alkylamine_{(C≤18)}, dialkylamine_{(C≤36)}, heterocycloalkane_{(C≤12)}, or a substituted version of any of these groups;
wherein the repeating unit comprises a degradable diacyl and a linker;
   the degradable diacyl group has the formula:

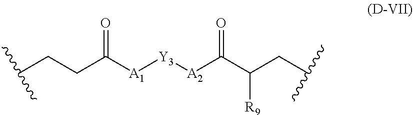

wherein:
   A₁ and A₂ are each independently —O—, —S—, or —NRₐ—, wherein:
   Rₐ is hydrogen, alkyl_{(C≤6)}, or substituted alkyl_{(C≤6)};
   Y₃ is alkanediyl_{(C≤12)}, alkenediyl_{(C≤12)}, arenediyl_{(C≤12)}, or a substituted version of any of these groups; or a group of the formula:

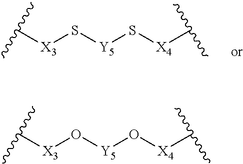

wherein:
   X₃ and X₄ are alkanediyl_{(C≤12)}, alkenediyl_{(C≤12)}, arenediyl_{(C≤12)}, or a substituted version of any of these groups;
   Y₅ is a covalent bond, alkanediyl_{(C≤12)}, alkenediyl_{(C≤12)}, arenediyl_{(C≤12)}, or a substituted version of any of these groups; and
   R₉ is alkyl_{(C≤8)} or substituted alkyl_{(C≤8)};
the linker group has the formula:

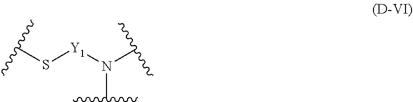

wherein:
   Y₁ is alkanediyl_{(C≤12)}, alkenediyl_{(C≤12)}, arenediyl_{(C≤12)}, or a substituted version of any of these groups; and
wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
the terminating group has the formula:

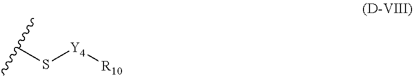

wherein:
   Y₄ is alkanediyl_{(C≤18)} or an alkanediyl_{(C≤18)} wherein one or more of the hydrogen atoms on the alkanediyl_{(C≤18)} has been replaced with —OH, —F, —Cl, —Br, —I, —SH, —OCH₃, —OCH₂CH₃, —SCH₃, or —OC(O)CH₃;
   R₁₀ is hydrogen, carboxy, hydroxy, or
   aryl_{(C≤12)}, alkylamino_{(C≤12)}, dialkylamino_{(C≤12)}, N-heterocycloalkyl_{(C≤12)}, —C(O)N(R₁₁)-alkanediyl_{(C≤6)}-heterocycloalkyl_{(C≤12)}, —C(O)-alkyl-amino_{(C≤12)}, —C(O)-dialkylamino_{(C≤12)}, —C(O)—N-heterocyclo-alkyl_{(C≤12)}, wherein:
   R₁₁ is hydrogen, alkyl_{(C≤6)}, or substituted alkyl_{(C≤6)};
   wherein the final degradable diacyl in the chain is attached to a terminating group;
   n is 0, 1, 2, 3, 4, 5, or 6;
      or a pharmaceutically acceptable salt thereof.

In some embodiments, the terminating group is further defined by the formula:

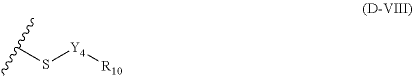

wherein:
Y₄ is alkanediyl_{(C≤18)}; and
R₁₀ is hydrogen.

In some embodiments, A₁ and A₂ are each independently —O— or —NRₐ—.

In some embodiments of the dendrimer or dendron of formula (D-I), the core is further defined by the formula:

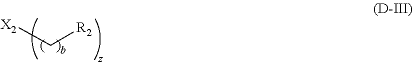

wherein:
X₂ is N(R₅)_{y};
   R₅ is hydrogen or alkyl_{(C≤8)}, or substituted alkyl_{(C≤18)}; and
   y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3.

In some embodiments of the dendrimer or dendron of formula (D-I), the core is further defined by the formula:

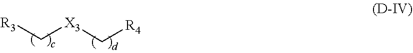

wherein:
X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

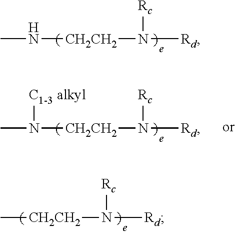

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
c and d are each independently 1, 2, 3, 4, 5, or 6.

In some embodiments of the dendrimer or dendron of formula (I), the terminating group is represented by the formula:

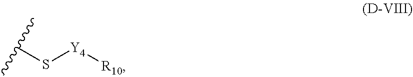

wherein:
Y₄ is alkanediyl_{(C≤18)}; and
R₁₀ is hydrogen.

In some embodiments of the dendrimer or dendron of formula (D-I), the core is further defined as:

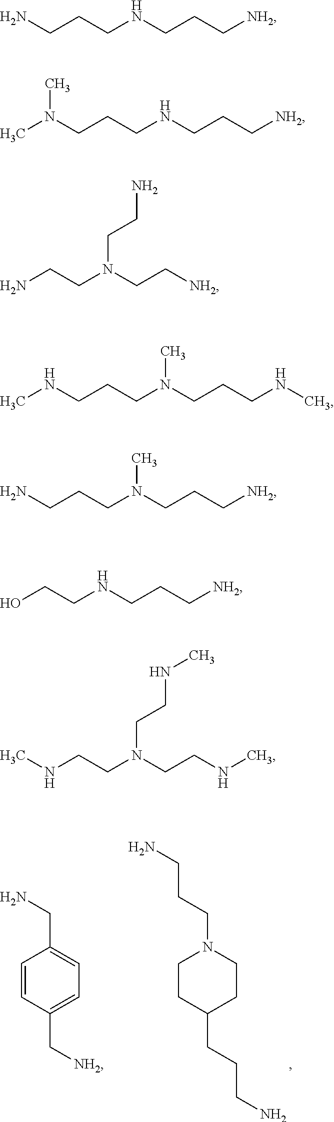

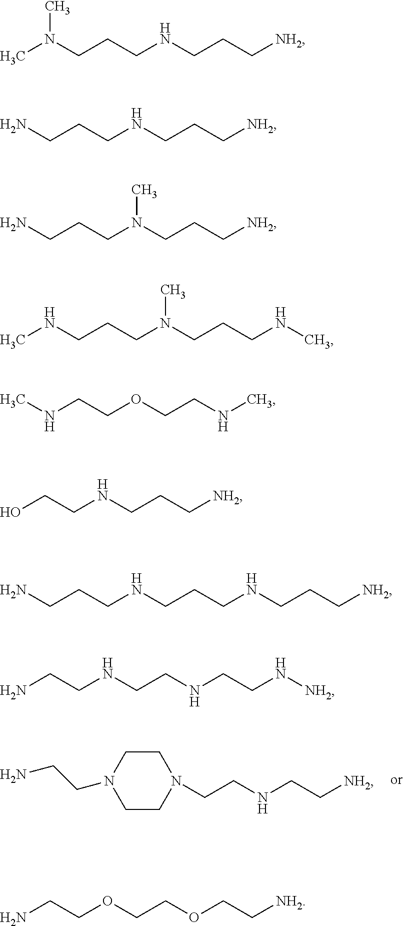

In some embodiments of the dendrimer or dendron of formula (D-I), the degradable diacyl is further defined as:

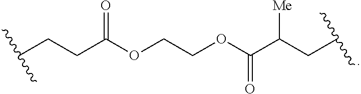 [SMILES: *CCC(=O)OCCOC(=O)C(C)C*]

In some embodiments of the dendrimer or dendron of formula (D-I), the linker is further defined as

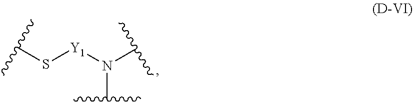

wherein Y₁ is alkanediyl_{(C≤8)} or substituted alkanediyl_{(C≤8)}.

In some embodiments of the dendrimer or dendron of formula (D-I), the dendrimer or dendron is selected from the group consisting of:

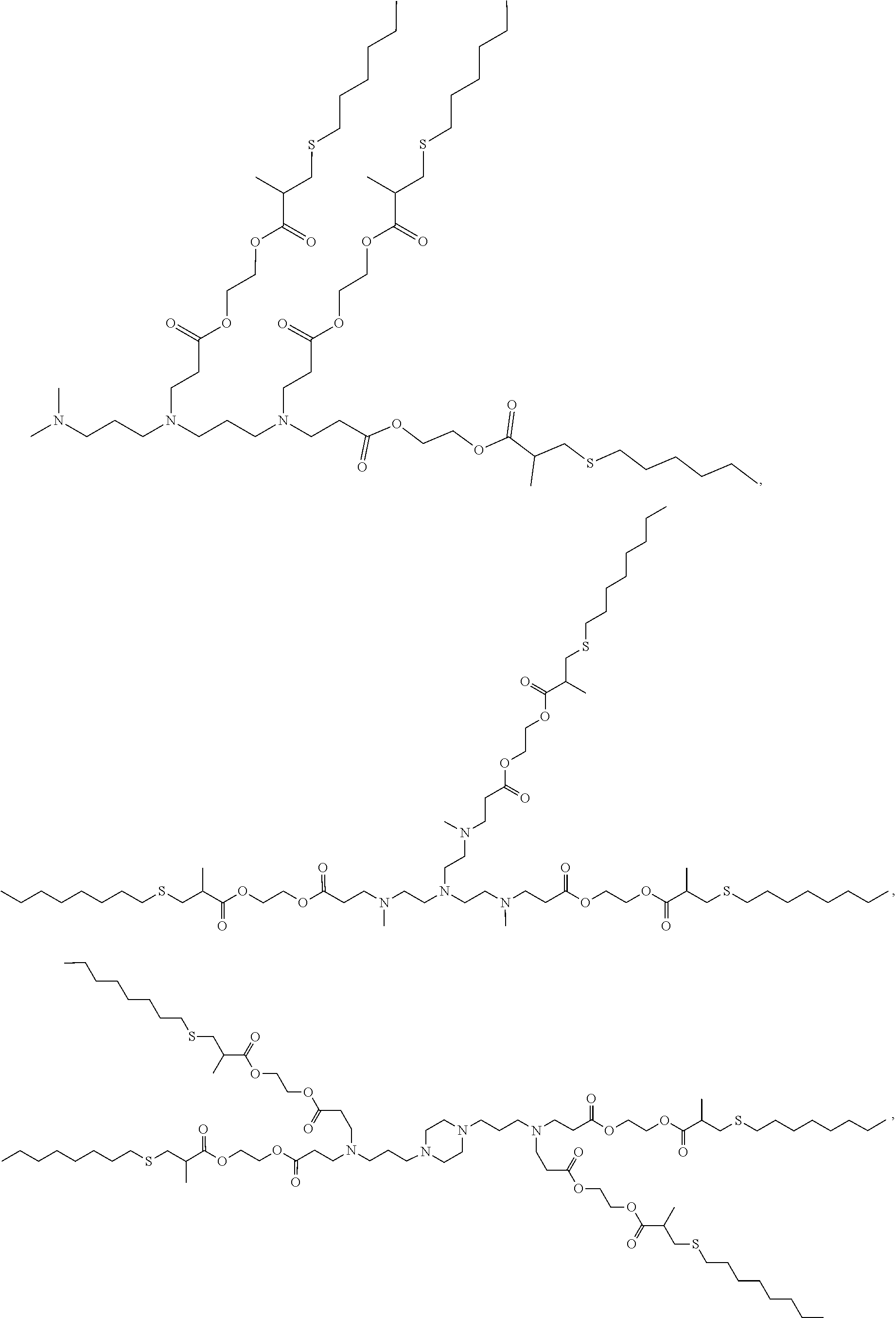

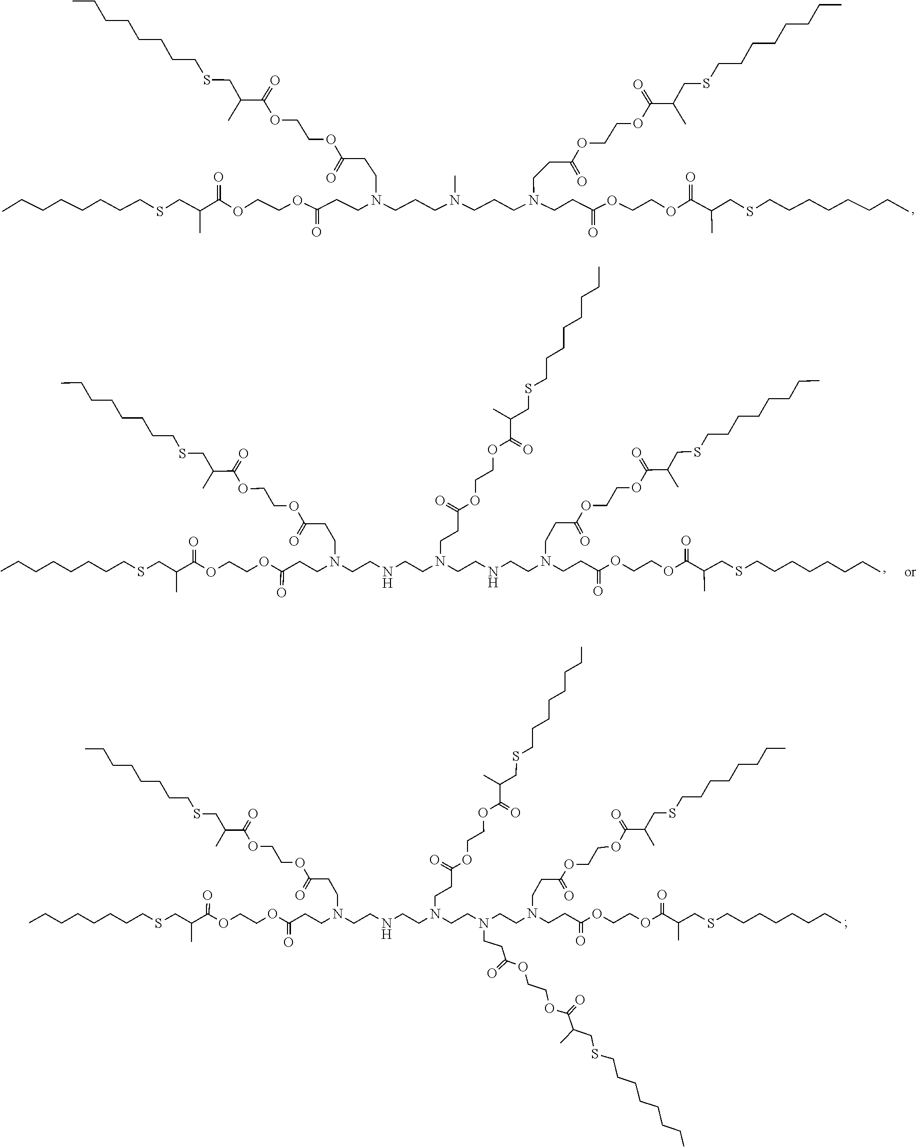

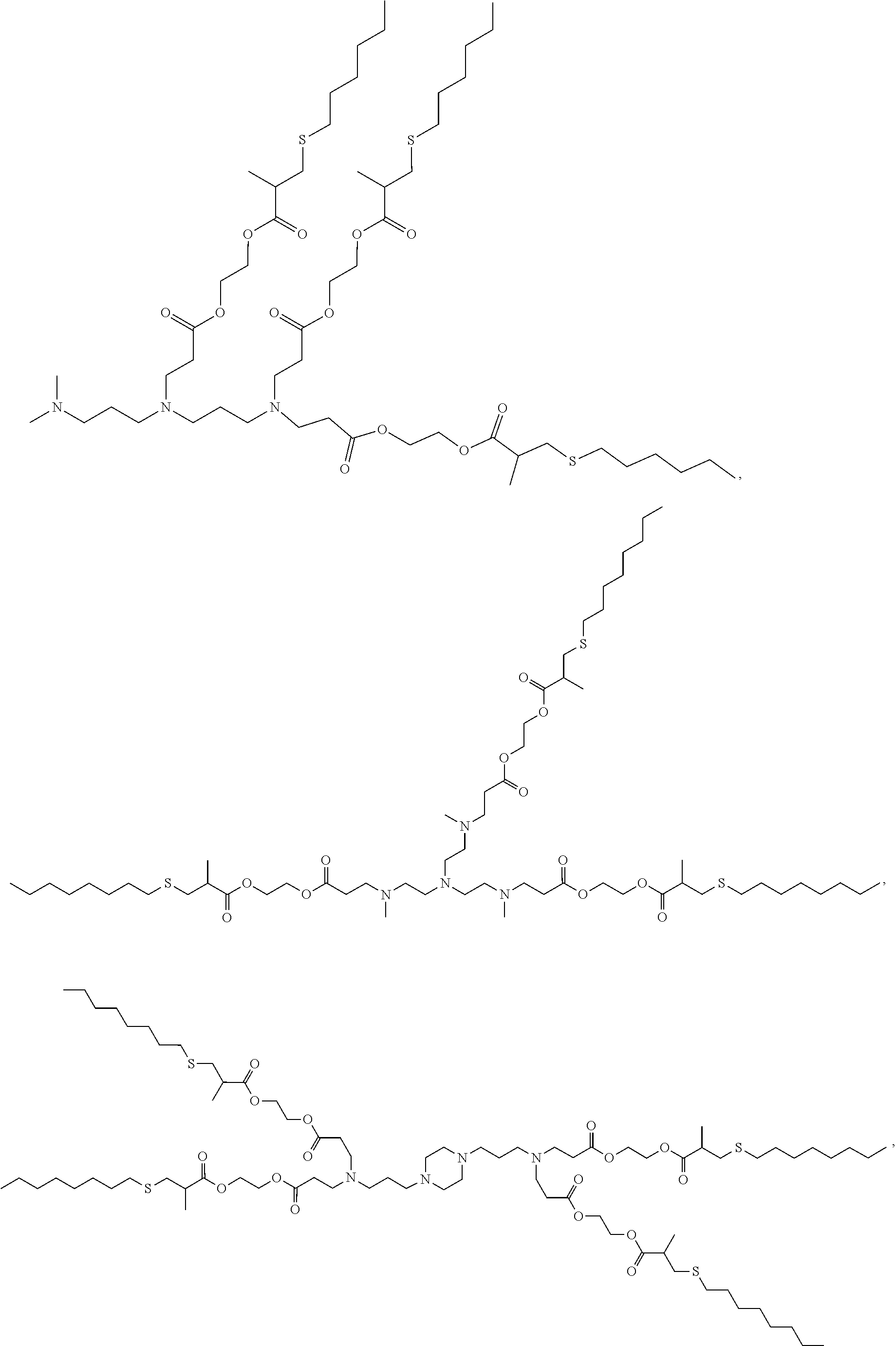

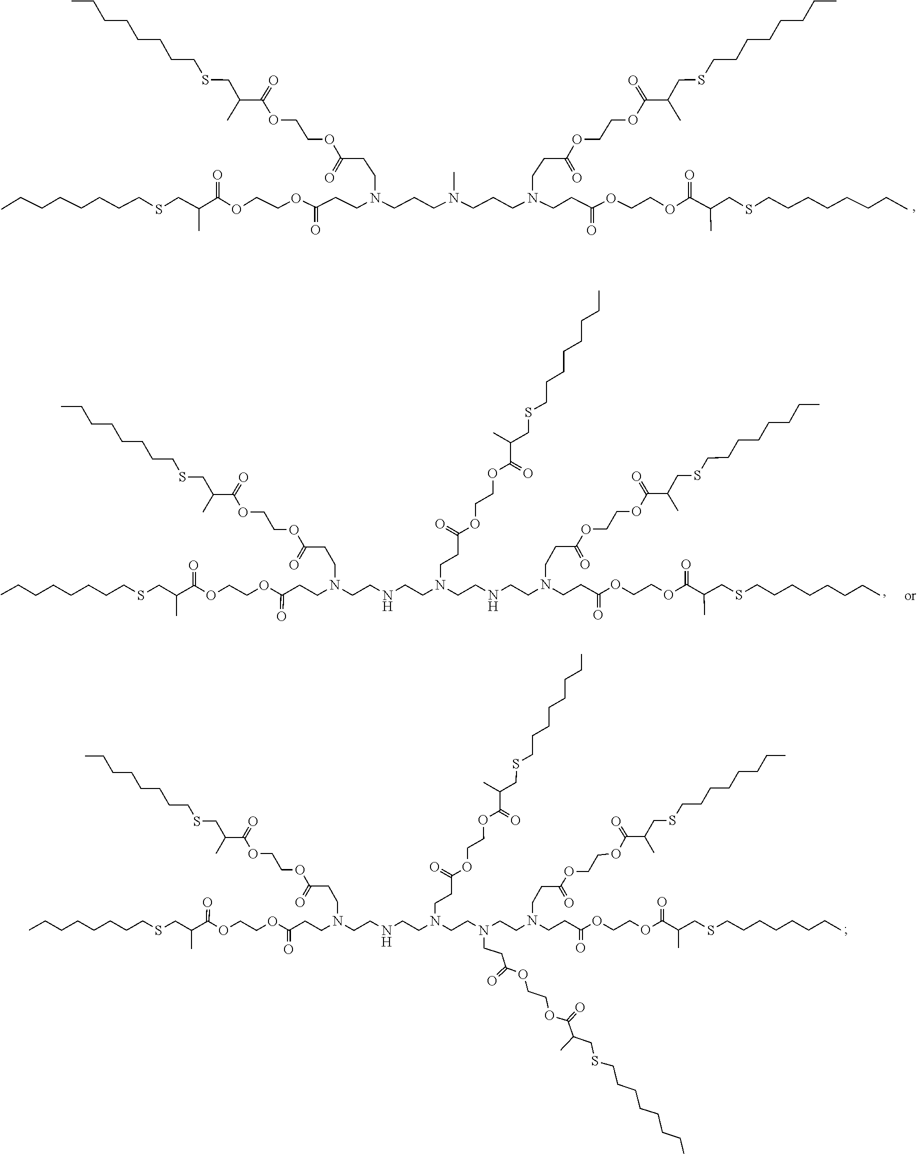

and pharmaceutically acceptable salts thereof.

### B. Dendrimers or Dendrons of Formula (X)

A. In some embodiments of the lipid composition, the ionizable cationic lipid is a dendrimer or dendron of the formula Core [symbol] Branch)_{N}. In some embodiments, the ionizable cationic lipid is a dendrimer or dendron of the formula

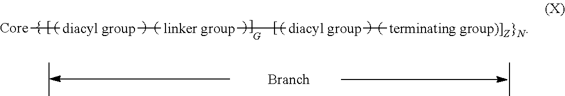

B. In some embodiments of the lipid composition, the ionizable cationic lipid is a dendrimer or dendron of a generation (g) having a structural formula:

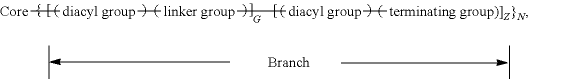

or a pharmaceutically acceptable salt thereof, wherein:
(a) the core comprises a structural formula (X_{Core}):

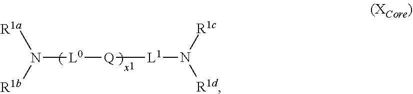

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

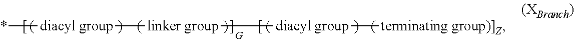

wherein:
   * indicates a point of attachment of the branch to the core;
   g is 1, 2, 3, or 4;
   Z=2^{(g-1)};
   G=0, when g=1; or G=Σᵢ₌₀^{i=g-2}2ⁱ, when g≠1;
(c) each diacyl group independently comprises a structural formula

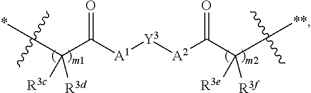

wherein:
   * indicates a point of attachment of the diacyl group at the proximal end thereof;
   ** indicates a point of attachment of the diacyl group at the distal end thereof;
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein:
      R⁴ is hydrogen or optionally substituted (e.g., C₁-C₆) alkyl;
   m¹ and m² are each independently at each occurrence 1, 2, or 3; and
   R^{3c}, R^{3d}, R^{3e} and R^{3f} are each independently at each occurrence hydrogen or an optionally substituted (e.g., C₁-C₈) alkyl; and
(d) each linker group independently comprises a structural formula

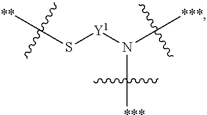

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

In some embodiments of X_{Core}, Q is independently at each occurrence a covalent bond, —O—, —S—, —NR²—, or —CR^{3a}R^{3b}. In some embodiments of X_{Core} Q is independently at each occurrence a covalent bond. In some embodiments of X_{Core} Q is independently at each occurrence an —O—. In some embodiments of X_{Core} Q is independently at each occurrence a —S—. In some embodiments of X_{Core} Q is independently at each occurrence a —NR² and R² is independently at each occurrence R^{1g} or -L²-NR^{1e}R^{1f}. In some embodiments of X_{Core} Q is independently at each occurrence a —CR^{3a}R^{3b}R^{3a}, and R^{3a} and R^{3b} are each independently at each occurrence hydrogen or an optionally substituted alkyl (e.g., C₁-C₆, such as C₁-C₃).

In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl. In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen. In some embodiments of X_{Core}, R^{1a}, R^{1b}, R^{1c}, R^{1d}, R^{1e}, R^{1f}, and R^{1g} (if present) are each independently at each occurrence a point of connection to a branch an optionally substituted alkyl (e.g., C₁-C₁₂).

In some embodiments of X_{Core}, L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or, alternatively, part of L¹ form a heterocycloalkyl (e.g., C₄-C₆ and containing one or two nitrogen atoms and, optionally, an additional heteroatom selected from oxygen and sulfur) with one of R^{1c} and R^{1d}. In some embodiments of X_{Core}, L⁰, L¹, and L² are each independently at each occurrence can be a covalent bond. In some embodiments of X_{Core}, L⁰, L¹, and L² are each independently at each occurrence can be a hydrogen. In some embodiments of X_{Core}, L⁰, L¹, and L² are each independently at each occurrence can be an alkylene (e.g., C₁-C₁₂, such as C₁-C₆ or C₁-C₃). In some embodiments of X_{Core}, L⁰, L¹, and L² are each independently at each occurrence can be a heteroalkylene (e.g., C₁-C₁₂, such as C₁-C₈ or C₁-C₆). In some embodiments of X_{Core}, L⁰, L¹, and L² are each independently at each occurrence can be a heteroalkylene (e.g., C₂-C₈ alkyleneoxide, such as oligo(ethyleneoxide)). In some embodiments of X_{Core}, L⁰, L¹, and L² are each independently at each occurrence can be a [alkylene]-[heterocycloalkyl]-[alkylene] [(e.g., C₁-C₆) alkylene]-[(e.g., C₄-C₆) heterocycloalkyl]-[(e.g., C₁-C₆) alkylene]. In some embodiments of X_{Core}, L⁰, L¹, and L² are each independently at each occurrence can be a [alkylene]-(arylene)-[alkylene] [(e.g., C₁-C₆) alkylene]-(arylene)-[(e.g., C₁-C₆) alkylene]. In some embodiments of X_{Core}, L⁰, L¹, and L² are each independently at each occurrence can be a [alkylene]-(arylene)-[alkylene] (e.g., [(e.g., C₁-C₆) alkylene]-phenylene-[(e.g., C₁-C₆) alkylene]). In some embodiments of X_{Core}, L⁰, L¹, and L² are each independently at each occurrence can be a heterocycloalkyl (e.g., C₄-C₆heterocycloalkyl). In some embodiments of X_{Core}, L⁰, L¹, and L² are each independently at each occurrence can be an arylene (e.g., phenylene). In some embodiments of X_{Core}, part of L¹ form a heterocycloalkyl with one of R^{1c} and R^{1d}. In some embodiments of X_{Core}, part of L¹ form a heterocycloalkyl (e.g., C₄-C₆ heterocycloalkyl) with one of R^{1c} and R^{1d} and the heterocycloalkyl can contain one or two nitrogen atoms and, optionally, an additional heteroatom selected from oxygen and sulfur.

In some embodiments of X_{Core}, L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

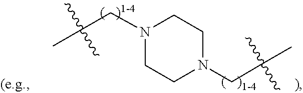

and [(C₁-C₄) alkylene]-phenylene-[(C₁-C₄) alkylene]

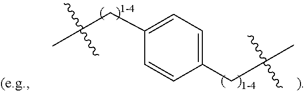

In some embodiments of X_{Core}, L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments of X_{Core}, L⁰, L¹, and L² are each independently at each occurrence C₁-C₆ alkylene (e.g., C₁-C₃ alkylene). In some embodiments, L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)). In some embodiments of X_{Core}, L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

In some embodiments of X_{Core}, x¹ is 0, 1, 2, 3, 4, 5, or 6. In some embodiments of X_{Core}, x¹ is 0. In some embodiments of X_{Core}, x¹ is 1. In some embodiments of X_{Core}, x¹ is 2. In some embodiments of X_{Core}, x¹ is 0, 3. In some embodiments of X_{Core} x¹ is 4. In some embodiments of X_{Core} x¹ is 5. In some embodiments of X_{Core}, x¹ is 6.

In some embodiments of X_{Core}, the core comprises a structural formula:

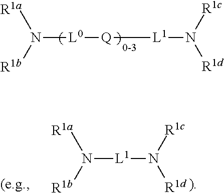

In some embodiments of X_{Core}, the core comprises a structural formula:

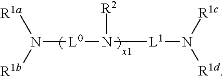

In some embodiments of X_{Core}, the core comprises a structural formula:

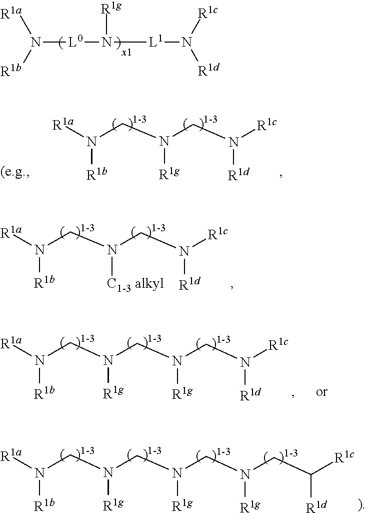

In some embodiments of X_{Core}, the core comprises a structural formula:

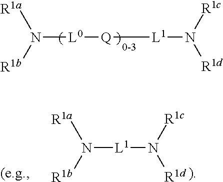

In some embodiments of X_{Core}, the core comprises a structural formula:

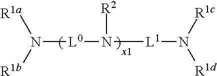

In some embodiments of X_{Core}, the core comprises a structural formula:

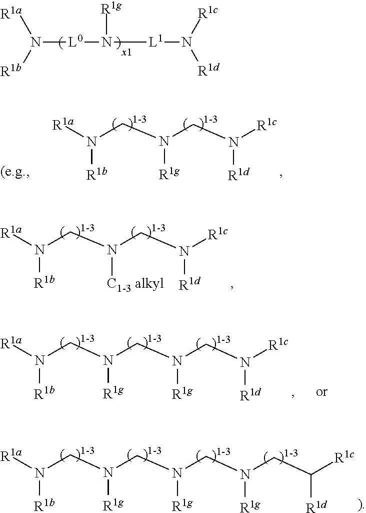

In some embodiments of X_{Core}, the core comprises a structural formula:

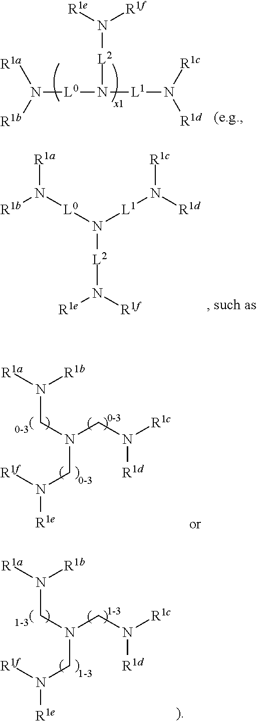

In some embodiments of X_{Core}, the core comprises a structural formula:

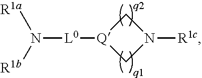

wherein Q′ is —NR2- or —CR3aR3b-; q1 and q2 are each independently 1 or 2. In some embodiments of X_{Core}, the core comprises a structural formula:

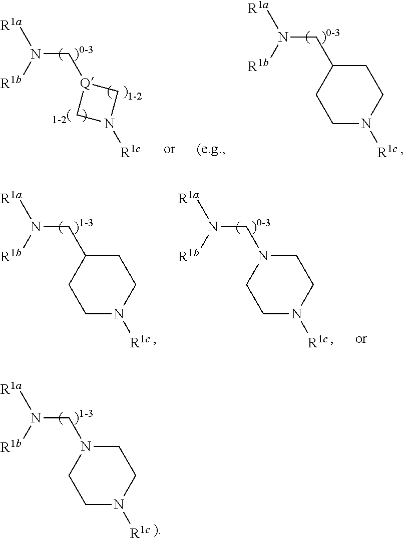

In some embodiments of X_{Core}, the core comprises a structural formula

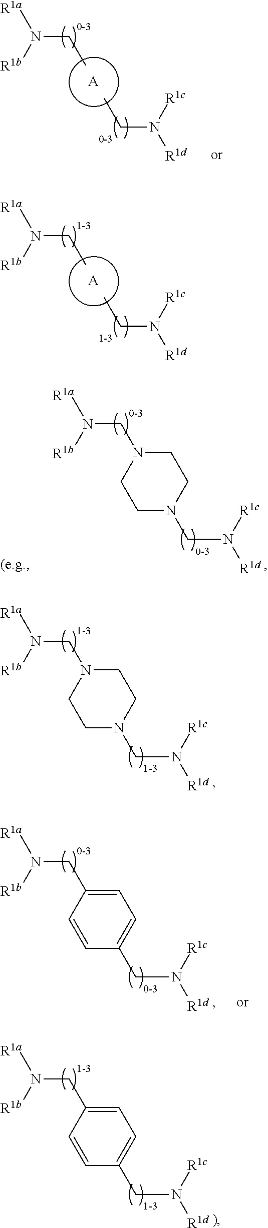

wherein ring A is an optionally substituted aryl or an optionally substituted (e.g., C3-C12, such as C3-C5) heteroaryl. In some embodiments of X_{Core}, the core comprises has a structural formula

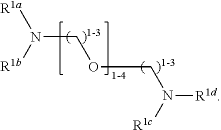

In some embodiments of X_{Core}, the core comprises a structural formula set forth in Table A and pharmaceutically acceptable salts thereof, wherein * indicates a point of attachment of the core to a branch of the plurality of branches. In some embodiments, the example cores of Table. A are not limited to the stereoisomers (i.e., enantiomers, diastereomers) listed.

TABLE A
Example core structures

| ID # | Structure |
|---|---|
| 1A1 | 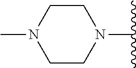 [SMILES: *N1CCN(C)CC1] |
| 1A2-1 | 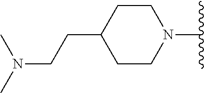 [SMILES: *N1CCC(CCN(C)C)CC1] |
| 1A2-2 | 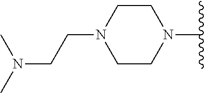 [SMILES: *N1CCN(CCN(C)C)CC1] |
| 1A3-1 | 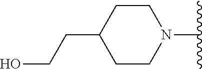 [SMILES: *N1CCC(CCO)CC1] |
| 1A3-2 | 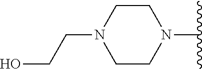 [SMILES: *N1CCN(CCO)CC1] |
| 1A4 | 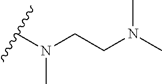 [SMILES: *N(C)CCN(C)C] |
| 1A5-1 | 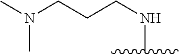 [SMILES: *NCCCN(C)C] |
| 1A5-2 | 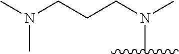 |
| 2A1-1 | 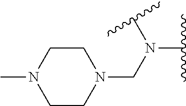 [SMILES: *N(*)CN1CCN(C)CC1] |
| 2A1-2 | 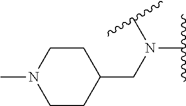 [SMILES: *N(*)CC1CCN(C)CC1] |
| 2A2-1 | 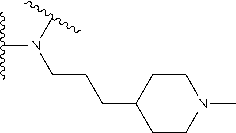 [SMILES: *N(*)CCCC1CCN(C)CC1] |
| 2A2-2 | 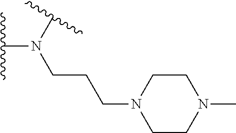 [SMILES: *N(*)CCCN1CCN(C)CC1] |
| 2A3 | 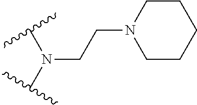 [SMILES: *N(*)CCN1CCCCC1] |
| 2A4 | 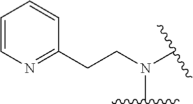 [SMILES: *N(*)CCc1ccccn1] |
| 2A5 | 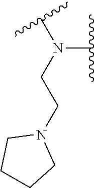 [SMILES: *N(*)CCN1CCCC1] |
| 2A6 | 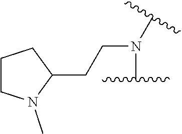 [SMILES: *N(*)CCC1CCCN1C] |
| 2A7-1 | 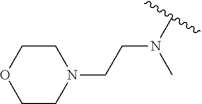 [SMILES: *N(C)CCN1CCOCC1] |
| 2A7-2 | 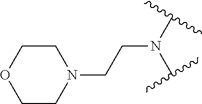 [SMILES: *N(*)CCN1CCOCC1] |
| 2A8 | 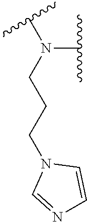 [SMILES: *N(*)CCCn1ccnc1] |
| 2A9 | 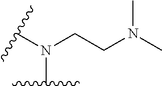 [SMILES: *N(*)CCN(C)C] |
| 2A9V | 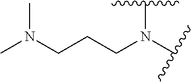 [SMILES: *N(*)CCCN(C)C] |
| 2A10 | 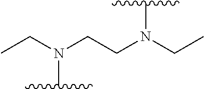 [SMILES: *N(CC)CCN(*)CC] |
| 2A11 | 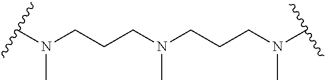 [SMILES: *N(C)CCCN(C)CCCN(*)C] |
| 2A12 | 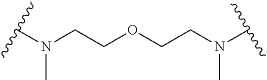 [SMILES: *N(C)CCOCCN(*)C] |
| 3A1 | 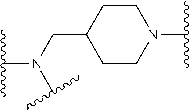 [SMILES: *N(*)CC1CCN(*)CC1] |
| 3A2 | 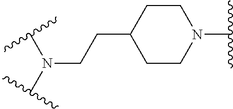 [SMILES: *N(*)CCC1CCN(*)CC1] |
| 3A3 | 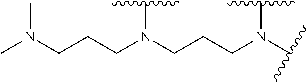 [SMILES: *N(*)CCCN(*)CCCN(C)C] |
| 3A4 | 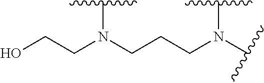 [SMILES: *N(*)CCCN(*)CCO] |
| 3A5 | 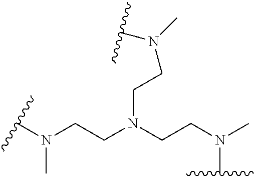 [SMILES: *N(C)CCN(CCN(*)C)CCN(*)C] |
| 3A6 | 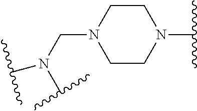 [SMILES: *N(*)CN1CCN(*)CC1] |
| 3A7 | 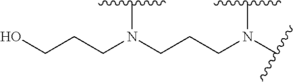 [SMILES: *N(*)CCCN(*)CCCO] |
| 4A1 | 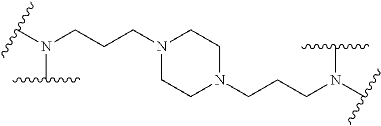 [SMILES: *N(*)CCCN1CCN(CCCN(*)*)CC1] |
| 4A2 | 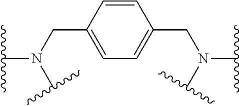 [SMILES: *N(*)Cc1ccc(CN(*)*)cc1] |
| 4A3 | 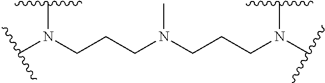 [SMILES: *N(*)CCCN(C)CCCN(*)*] |
| 4A4 | 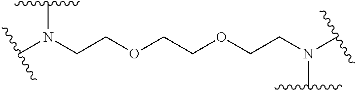 [SMILES: *N(*)CCOCCOCCN(*)*] |
| 5A1 | 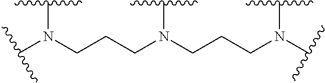 [SMILES: *N(*)CCCN(*)CCCN(*)*] |
| 5A2-1 (5-arm) | 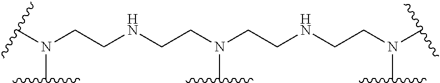 [SMILES: *N(*)CCNCCN(*)CCNCCN(*)*] |
| 5A2-2 (5-arm) | 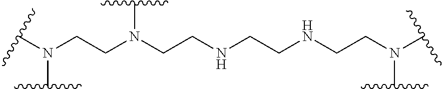 [SMILES: *N(*)CCNCCNCCN(*)CCN(*)*] |
| 5A2-3 (5-arm) | 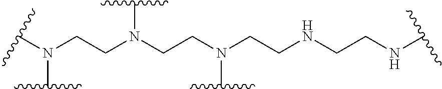 [SMILES: *NCCNCCN(*)CCN(*)CCN(*)*] |
| 5A2-4 (5-arm) | 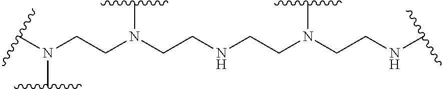 [SMILES: *NCCN(*)CCNCCN(*)CCN(*)*] |
| 5A3-1 (5-arm) | 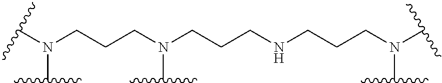 [SMILES: *N(*)CCCNCCCN(*)CCCN(*)*] |
| 5A4-1 (5-arm) | 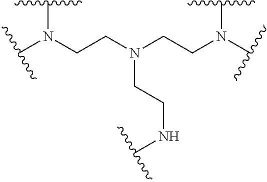 [SMILES: *NCCN(CCN(*)*)CCN(*)*] |
| 5A5 | 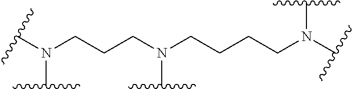 [SMILES: *N(*)CCCCN(*)CCCN(*)*] |
| 5A6 | 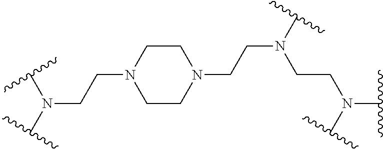 [SMILES: *N(*)CCN(*)CCN1CCN(CCN(*)*)CC1] |
| 5A2-4 (6-arm) | 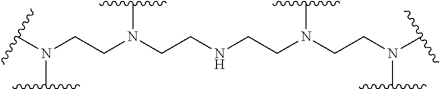 [SMILES: *N(*)CCN(*)CCNCCN(*)CCN(*)*] |
| 5A2-5 (6 arm) | 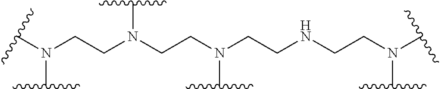 [SMILES: *N(*)CCNCCN(*)CCN(*)CCN(*)*] |
| 5A2-6 (6 arm) | 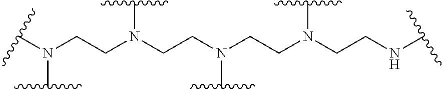 [SMILES: *NCCN(*)CCN(*)CCN(*)CCN(*)*] |
| 6A2 | 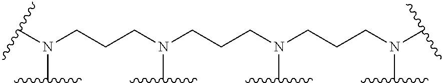 [SMILES: *N(*)CCCN(*)CCCN(*)CCCN(*)*] |
| 5A3-2 (6 arm) | 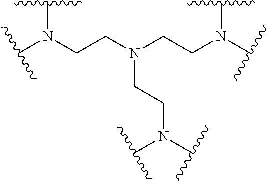 [SMILES: *N(*)CCN(CCN(*)*)CCN(*)*] |
| 5A4-2 (6 arm) | 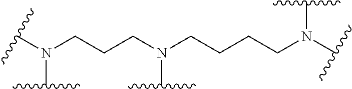 [SMILES: *N(*)CCCCN(*)CCCN(*)*] |
| 6A4 | 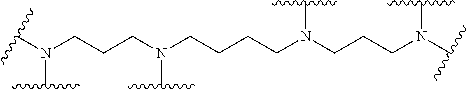 [SMILES: *N(*)CCCN(*)CCCCN(*)CCCN(*)*] |
| 1H1 | 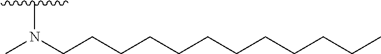 [SMILES: *N(C)CCCCCCCCCCCC] |
| 1H2 | 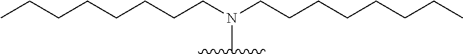 [SMILES: *N(CCCCCCCC)CCCCCCCC] |
| 1H3 | 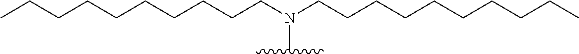 [SMILES: *N(CCCCCCCCCC)CCCCCCCCCC] |
| 2H1 | 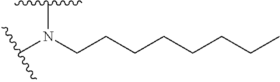 [SMILES: *N(*)CCCCCCCC] |
| 2H2 | 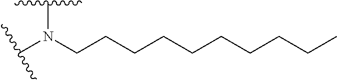 [SMILES: *N(*)CCCCCCCCCC] |
| 2H3 | 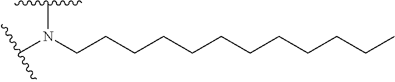 [SMILES: *N(*)CCCCCCCCCCCC] |
| 2H4 | 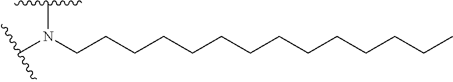 [SMILES: *N(*)CCCCCCCCCCCCCC] |
| 2H5 | 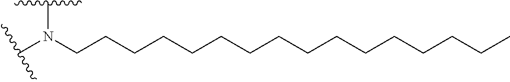 [SMILES: *N(*)CCCCCCCCCCCCCCCC] |
| 2H6 | 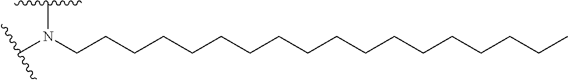 [SMILES: *N(*)CCCCCCCCCCCCCCCCCC] |

In some embodiments of X_{Core}, the core comprises a structural formula selected from the group consisting of:

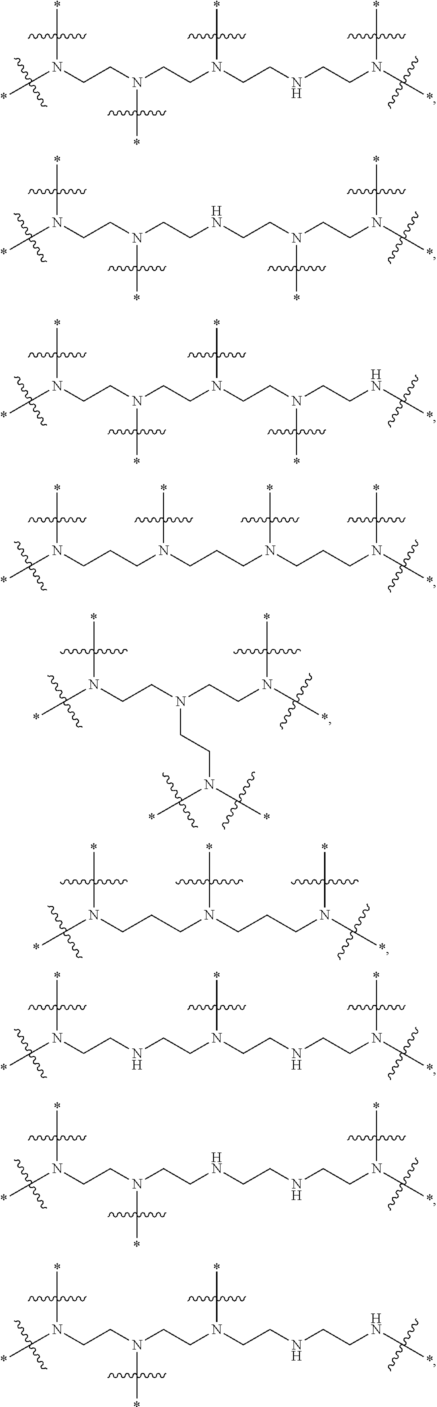

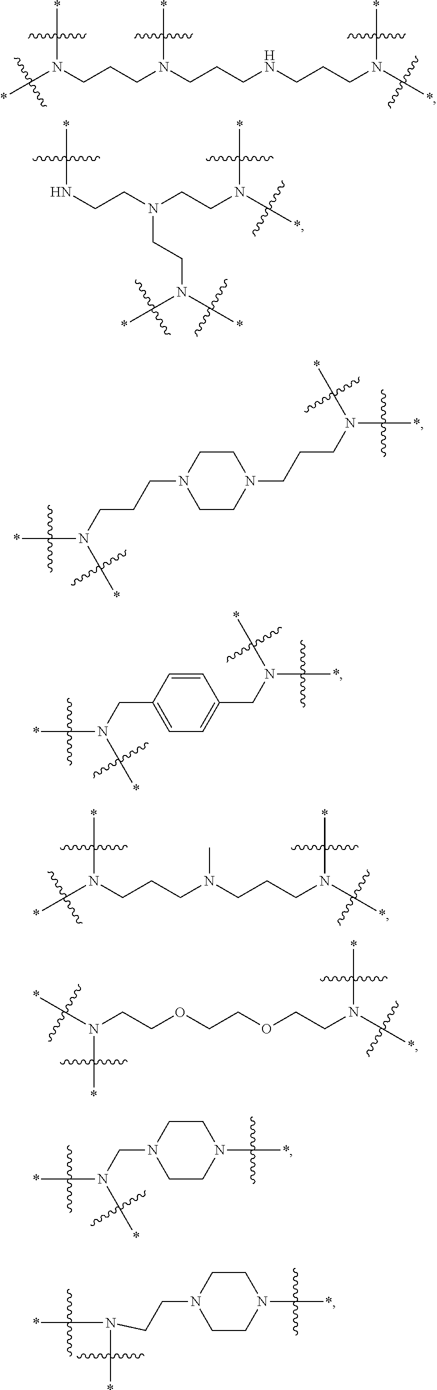

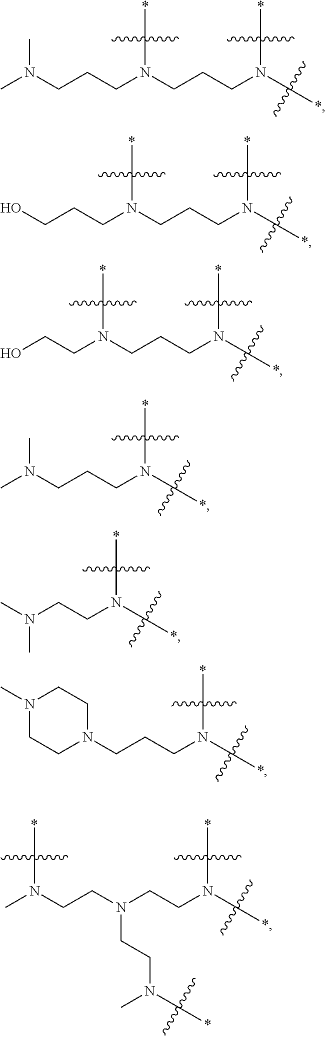

and pharmaceutically acceptable salts thereof, wherein * indicates a point of attachment of the core to a branch of the plurality of branches or H. In some embodiments, wherein * indicates a point of attachment of the core to a branch of the plurality of branches.

In some embodiments of X_{Core}, the core has the structure

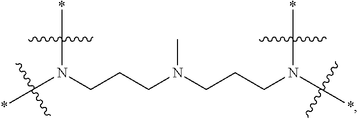

wherein * indicates a point of attachment of the core to a branch of the plurality of branches or H. In some embodiments, at least 2 branches are attached to the core. In some embodiments, at least 3 branches are attached to the core. In some embodiments, at least 4 branches are attached to the core.

In some embodiments of X_{Core}, the core has the structure

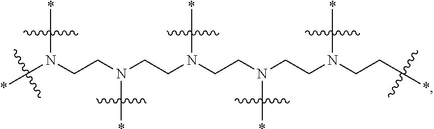

wherein * indicates a point of attachment of the core to a branch of the plurality of branches or H. In some embodiments, at least 4 branches are attached to the core. In some embodiments, at least 5 branches are attached to the core. In some embodiments, at least 6 branches are attached to the core.

In some embodiments, the plurality (N) of branches comprises at least 3 branches, at least 4 branches, at least 5 branches. In some embodiments, the plurality (N) of branches comprises at least 3 branches. In some embodiments, the plurality (N) of branches comprises at least 4 branches. In some embodiments, the plurality (N) of branches comprises at least 5 branches.

In some embodiments of X_{Branch}, g is 1, 2, 3, or 4. In some embodiments of X_{Branch}, g is 1. In some embodiments of X_{Branch}, g is 2. In some embodiments of X_{Branch}, g is 3. In some embodiments of X_{Branch}, g is 4.

In some embodiments of XBranch, Z=2(g−1) and when g=1, G=0. In some embodiments of X_{Branch}, Z=2(g−1) and G=Σᵢ₌₀^{i=g-2}2i, when g≠1.

In some embodiments of X_{Branch}, g=1, G=0, Z=1, and each branch of the plurality of branches comprises a structural formula each branch of the plurality of branches

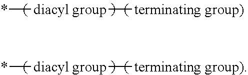

In some embodiments of X_{Branch}, g=2, G=1, Z=2, and each branch of the plurality of branches comprises a structural formula

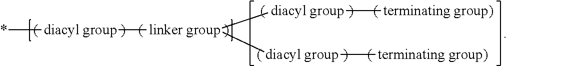

In some embodiments of X_{Branch}, g=3, G=3, Z=4, and each branch of the plurality of branches comprises a structural formula

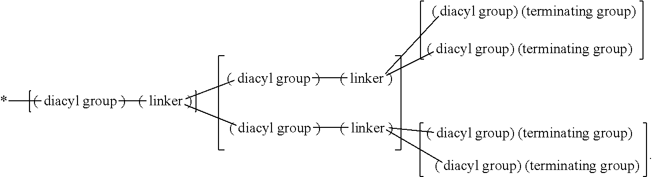

In some embodiments of X_{Branch}, g=4, G=7, Z=8, and each branch of the plurality of branches comprises a structural formula

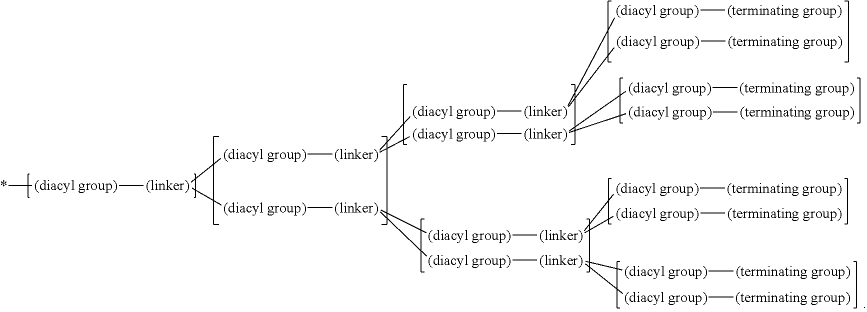

In some embodiments, the dendrimers or dendrons described herein with a generation (g)=1 has the structure:

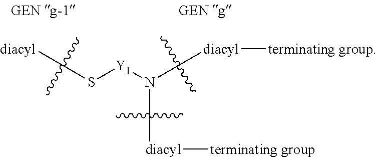

In some embodiments, the dendrimers or dendrons described herein with a generation (g)=1 has the structure:

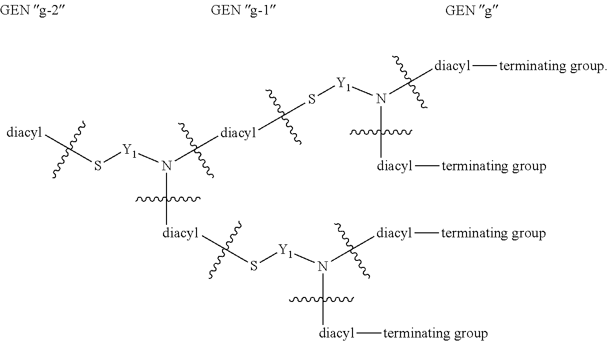

An example formulation of the dendrimers or dendrons described herein for generations 1-4 is shown in Table B. The number of diacyl groups, linker groups, and terminating groups can be calculated based on g.

TABLE B
Formulation of Dendrimer or Dendron Groups Based on Generation (g)

| | g = 1 | g = 2 | g = 3 | g = 4 | |
|---|---|---|---|---|---|
| # of diacyl | 1 | 1 + | 1 + 2 + | 1 + 2 + 2² + | 1 + 2 + . . . + |
| grp | | 2 = 3 | 2² = 7 | 2³ = 15 | 2^{g-1} |
| # of linker | 0 | 1 | 1 + 2 | 1 + 2 + 2² | 1 + 2 + . . . + |
| grp | | | | | 2^{g-2} |
| # of | 1 | 2 | 2² | 2³ | 2^{(g-1)} |
| terminating | | | | | |
| grp | | | | | |

In some embodiments, the diacyl group independently comprises a structural formula

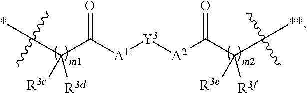

* indicates a point of attachment of the diacyl group at the proximal end thereof, and ** indicates a point of attachment of the diacyl group at the distal end thereof.

In some embodiments of the diacyl group of X_{Branch}, Y³ is independently at each occurrence an optionally substituted; alkylene, an optionally substituted alkenylene, or an optionally substituted arenylene. In some embodiments of the diacyl group of X_{Branch}, Y³ is independently at each occurrence an optionally substituted alkylene (e.g., C₁-C₁₂). In some embodiments of the diacyl group of X_{Branch}, Y³ is independently at each occurrence an optionally substituted alkenylene (e.g., C₁-C₁₂). In some embodiments of the diacyl group of X_{Branch}, Y³ is independently at each occurrence an optionally substituted arenylene (e.g., C₁-C₁₂).

In some embodiments of the diacyl group of X_{Branch}, A¹ and A² are each independently at each occurrence —O—, —S—, or —NR⁴—. In some embodiments of the diacyl group of X_{Branch}, A¹ and A² are each independently at each occurrence —O—. In some embodiments of the diacyl group of X_{Branch}, A¹ and A² are each independently at each occurrence —S—. In some embodiments of the diacyl group of X_{Branch}, A¹ and A² are each independently at each occurrence —NR⁴— and R⁴ is hydrogen or optionally substituted alkyl (e.g., C₁-C₆). In some embodiments of the diacyl group of X_{Branch}, m¹ and m² are each independently at each occurrence 1, 2, or 3. In some embodiments of the diacyl group of X_{Branch}, m¹ and m² are each independently at each occurrence 1. In some embodiments of the diacyl group of X_{Branch}, m¹ and m² are each independently at each occurrence 2. In some embodiments of the diacyl group of X_{Branch}, m¹ and m² are each independently at each occurrence 3. In some embodiments of the diacyl group of X_{Branch}, R^{3c}, R^{3d}, R^{3e}, and R^{3f} are each independently at each occurrence hydrogen or an optionally substituted alkyl. In some embodiments of the diacyl group of X_{Branch}, R^{3c}, R^{3d}, R^{3e}, and R^{3f} are each independently at each occurrence hydrogen. In some embodiments of the diacyl group of X_{Branch}, R^{3c}, R^{3d}, R^{3e}, and R^{3f} are each independently at each occurrence an optionally substituted (e.g., C₁-C₈) alkyl.

In some embodiments of the diacyl group, A¹ is —O— or —NH—. In some embodiments of the diacyl group, A¹ is —O—. In some embodiments of the diacyl group, A² is —O— or —NH—. In some embodiments of the diacyl group, A² is —O—. In some embodiments of the diacyl group, Y³ is C₁-C₁₂ (e.g., C₁-C₆, such as C₁-C₃) alkylene.

In some embodiments of the diacyl group, the diacyl group independently at each occurrence comprises a structural formula

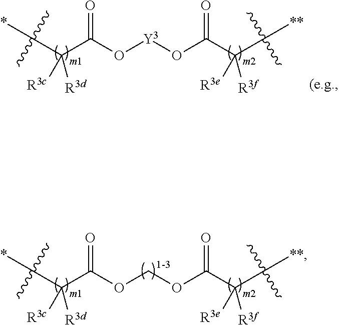

such as

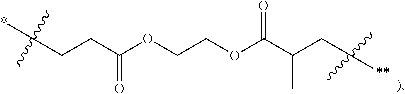

and optionally R^{3c}, R^{3d}, R^{3e}, and R^{3f} are each independently at each occurrence hydrogen or C₁-C₃ alkyl.

In some embodiments, linker group independently comprises a structural formula

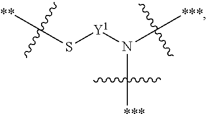

** indicates a point of attachment of the linker to a proximal diacyl group, and *** indicates a point of attachment of the linker to a distal diacyl group.

In some embodiments of the linker group of X_{Branch} if present, Y₁ is independently at each occurrence an optionally substituted alkylene, an optionally substituted alkenylene, or an optionally substituted arenylene. In some embodiments of the linker group of X_{Branch} if present, Y₁ is independently at each occurrence an optionally substituted alkylene (e.g., C₁-C₁₂). In some embodiments of the linker group of X_{Branch} if present, Y1 is independently at each occurrence an optionally substituted alkenylene (e.g., C₁-C₁₂). In some embodiments of the linker group of X_{Branch} if present, Y₁ is independently at each occurrence an optionally substituted arenylene (e.g., C₁-C₁₂).

In some embodiments of the terminating group of X_{Branch}, each terminating group is independently selected from optionally substituted alkylthiol and optionally substituted alkenylthiol. In some embodiments of the terminating group of X_{Branch}, each terminating group is an optionally substituted alkylthiol (e.g., C₁-C₁₈, such as C₄-C₁₈). In some embodiments of the terminating group of X_{Branch}, each terminating group is optionally substituted alkenylthiol (e.g., C₁-C₁₈, such as C₄-C₁₈).

In some embodiments of the terminating group of X_{Branch}, each terminating group is independently C₁-C₁₈ alkenylthiol or C₁-C₁₈ alkylthiol, and the alkyl or alkenyl moiety is optionally substituted with one or more substituents each independently selected from halogen, C₆-C₁₂ aryl, C₁-C₁₂ alkylamino, C₄-C₆ N-heterocycloalkyl, —OH, —C(O)OH, —C(O)N(C₁-C₃ alkyl)-(C₁-C₆ alkylene)-(C₁-C₁₂ alkylamino), —C(O)N(C₁-C₃ alkyl)-(C₁-C₆ alkylene)-(C₄-C₆ N-heterocycloalkyl), —C(O)—(C₁-C₁₂ alkylamino), and —C(O)—(C₄-C₆ N-heterocycloalkyl), and the C₄-C₆ N-heterocycloalkyl moiety of any of the preceding substituents is optionally substituted with C₁-C₃ alkyl or C₁-C₃ hydroxyalkyl.

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

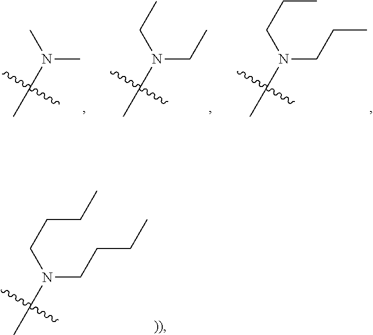

C₄-C₆ N-heterocycloalkyl (e.g., N-pyrrolidinyl

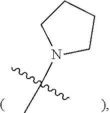 [SMILES: *N1CCCC1]

### N-piperidinyl

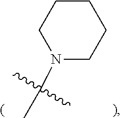 [SMILES: *N1CCCCC1]

### N-azepanyl

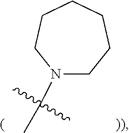 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

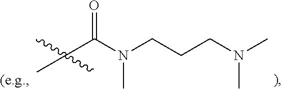 [SMILES: *C(=O)N(C)CCCN(C)C]

—C(O)N(C₁-C₃ alkyl)-(C₁-C₆ alkylene)-(C₄-C₆ N-heterocycloalkyl

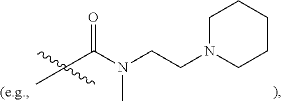 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

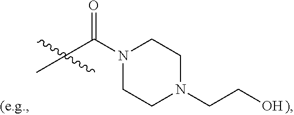 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl. In some embodiments of the terminating group of X_{Branch}, each terminating group is independently C₁-C₁₈ (e.g., C₄-C₁₈) alkylthiol, wherein the alkyl moiety is optionally substituted with one substituent —OH. In some embodiments of the terminating group of X_{Branch}, each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

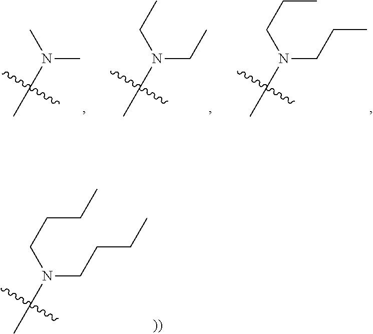

and C₄-C₆ N-heterocycloalkyl (e.g., N-pyrrolidinyl

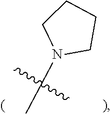 [SMILES: *N1CCCC1]

### N-piperidinyl

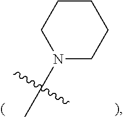 [SMILES: *N1CCCCC1]

### N-azepanyl

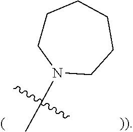 [SMILES: *N1CCCCCC1]

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol. In some embodiments of the terminating group of X_{Branch}, each terminating group is independently C₁-C₁₈ (e.g., C₄-C₁₈) alkylthiol.

In some embodiments of the terminating group of X_{Branch}, each terminating group is independently a structural set forth in Table C. In some embodiments, the dendrimers or dendrons described herein can comprise a terminating group or pharmaceutically acceptable salt, or thereof selected in Table C. In some embodiments, the example terminating group of Table C are not limiting of the stereoisomers (i.e., enantiomers, diastereomers) listed.

TABLE C
Example terminating group / peripheries structures

| ID # | Structure |
|---|---|
| SC1 | 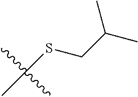 [SMILES: *SCC(C)C] |
| SC2 | 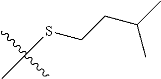 [SMILES: *SCCC(C)C] |
| SC3 | 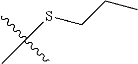 [SMILES: *SCCC] |
| SC4 | 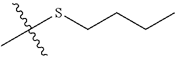 [SMILES: *SCCCC] |
| SC5 | 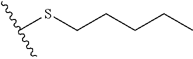 [SMILES: *SCCCCC] |
| SC6 | 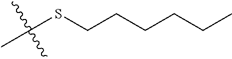 [SMILES: *SCCCCCC] |
| SC7 | 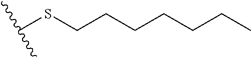 [SMILES: *SCCCCCCC] |
| SC8 | 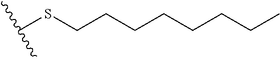 [SMILES: *SCCCCCCCC] |
| SC9 | 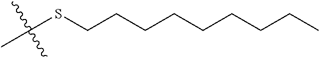 [SMILES: *SCCCCCCCCC] |
| SC10 | 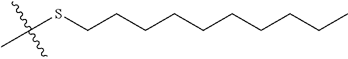 [SMILES: *SCCCCCCCCCC] |
| SC11 | 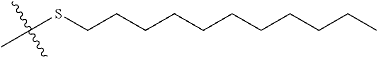 [SMILES: *SCCCCCCCCCCC] |
| SC12 | 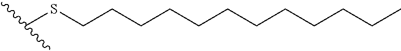 [SMILES: *SCCCCCCCCCCCC] |
| SC14 | 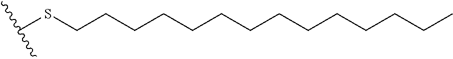 [SMILES: *SCCCCCCCCCCCCCC] |
| SC16 | 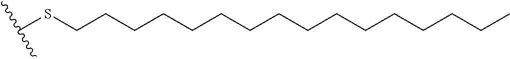 [SMILES: *SCCCCCCCCCCCCCCCC] |
| SC18 | 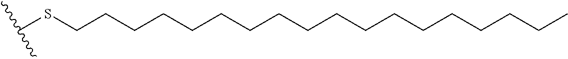 [SMILES: *SCCCCCCCCCCCCCCCCCC] |
| SC19 | 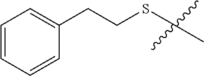 [SMILES: *SCCc1ccccc1] |
| SO1 | 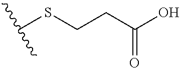 [SMILES: *SCCC(=O)O] |
| SO2 | 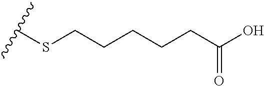 [SMILES: *SCCCCCC(=O)O] |
| SO3 | 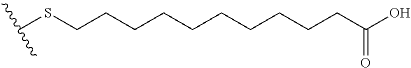 [SMILES: *SCCCCCCCCCCC(=O)O] |
| SO4 | 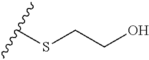 [SMILES: *SCCO] |
| SO5 | 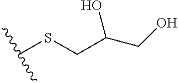 [SMILES: *SCC(O)CO] |
| SO6 | 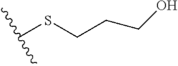 [SMILES: *SCCCO] |
| SO7 | 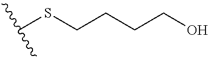 [SMILES: *SCCCCO] |
| SO8 | 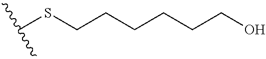 [SMILES: *SCCCCCCO] |
| SO9 | 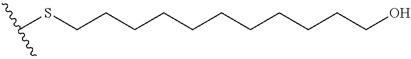 [SMILES: *SCCCCCCCCCCCO] |
| SN1 | 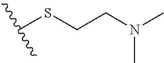 [SMILES: *SCCN(C)C] |
| SN2 | 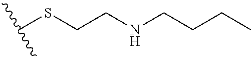 [SMILES: *SCCNCCCC] |
| SN3 | 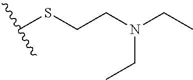 [SMILES: *SCCN(CC)CC] |
| SN4 | 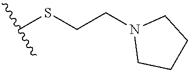 [SMILES: *SCCN1CCCC1] |
| SN5 | 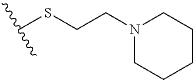 [SMILES: *SCCN1CCCCC1] |
| SN6 | 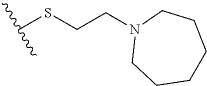 [SMILES: *SCCN1CCCCCC1] |
| SN7 | 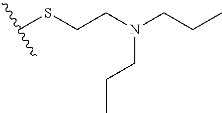 [SMILES: *SCCN(CCC)CCC] |
| SN8 | 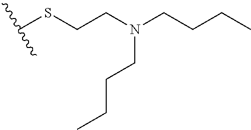 [SMILES: *SCCN(CCCC)CCCC] |
| SN9 | 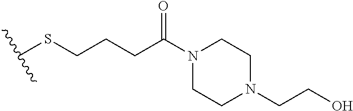 [SMILES: *SCCCC(=O)N1CCN(CCO)CC1] |
| SN10 | 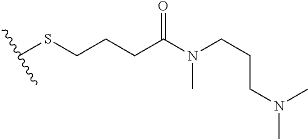 [SMILES: *SCCCC(=O)N(C)CCCN(C)C] |
| SN11 | 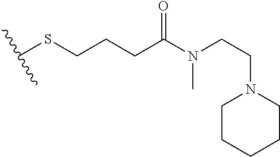 [SMILES: *SCCCC(=O)N(C)CCN1CCCCC1] |

In some embodiments, the dendrimer or dendron of Formula (X) is selected from those set forth in Table D and pharmaceutically acceptable salts thereof.

TABLE D
Example Ionizable Cationic Lipo-dendrimers

| ID # | Structure |
|---|---|
| 2A2- SC14 | 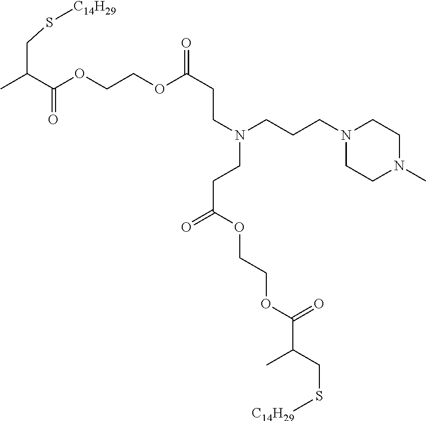 |
| 2A6- SC14 | 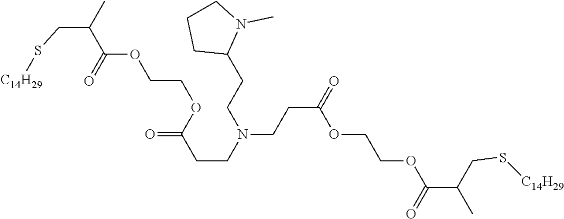 |
| 2A9- SC14 | 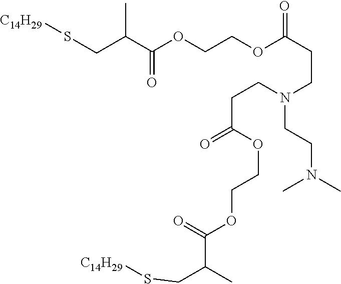 |
| 3A3- SC10 | 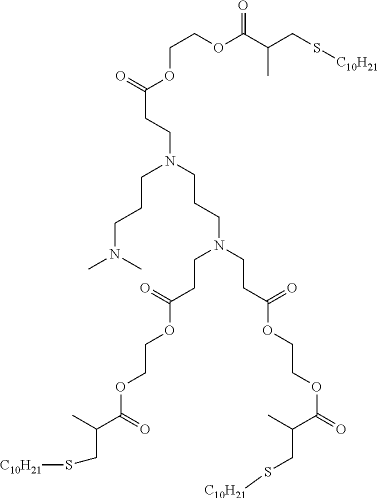 |
| 3A3- SC14 | 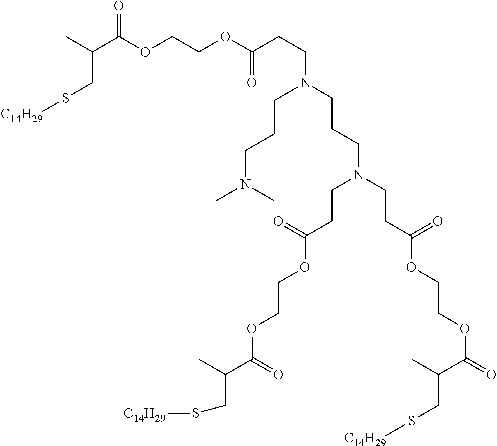 |
| 3A5- SC10 | 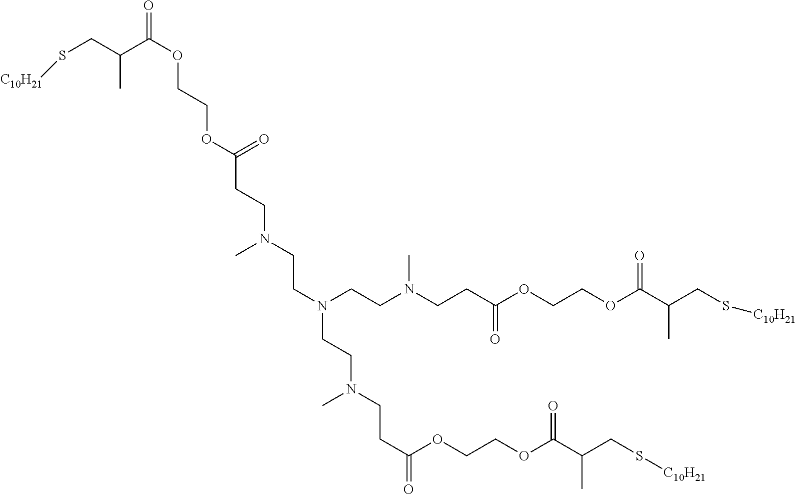 |
| 3A5- SC14 | 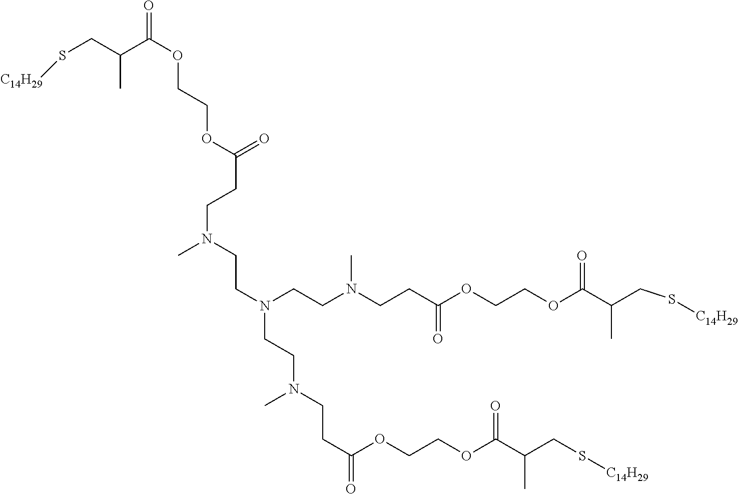 |
| 4A1- SC12 | 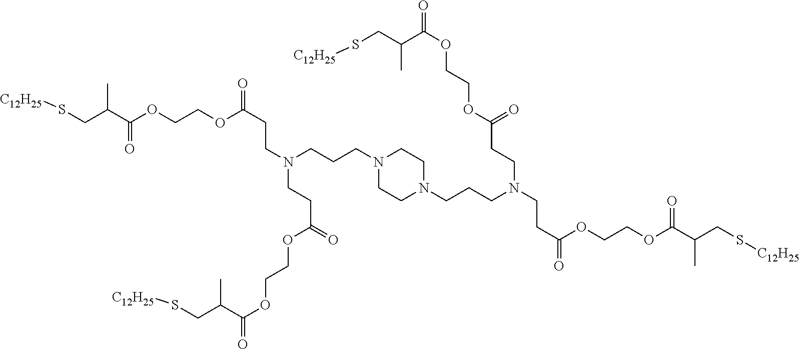 |
| 4A3- SC12 | 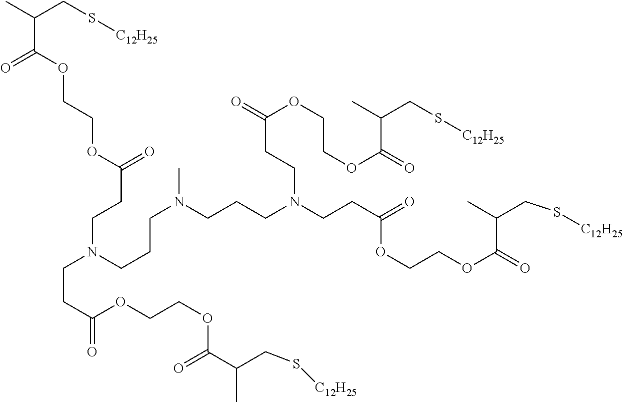 |
| 5A1- SC12 | 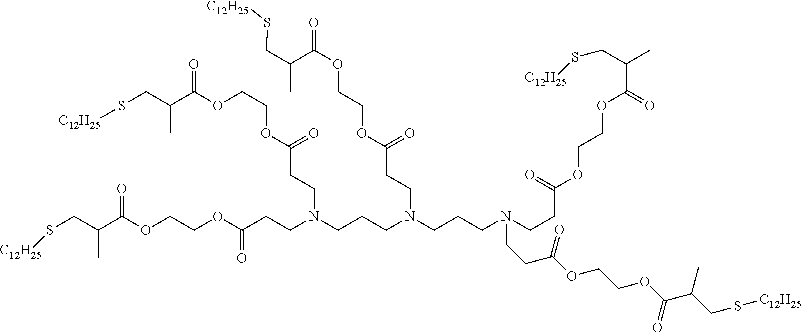 |
| 5A1- SC8 | 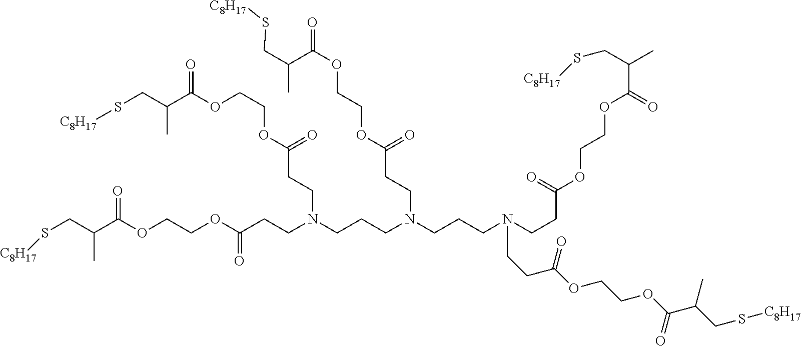 |
| 5A2- 2- SC12 (5- arm) | 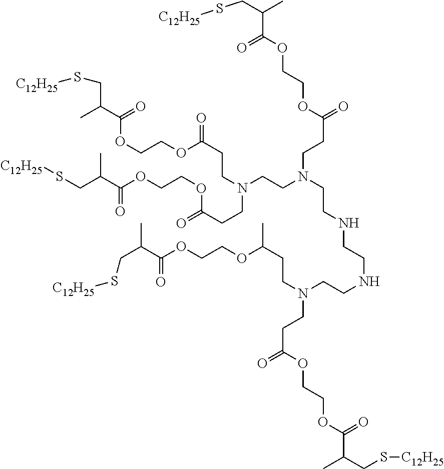 |
| 5A3- 1- SC12 (5 arm) | 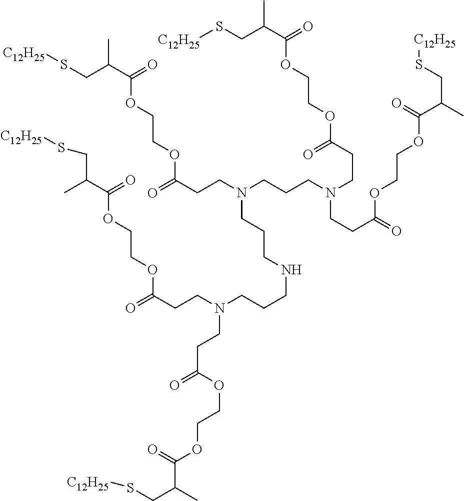 |
| 5A3- 1-SC8 (5- arm) | 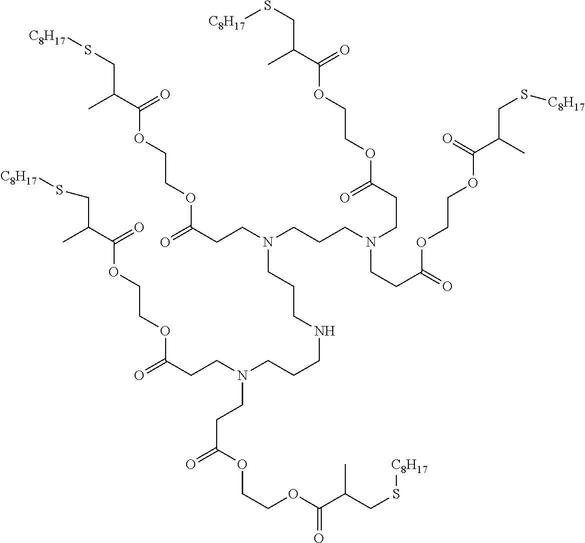 |
| 5A4- 1- SC12 (5- arm) | 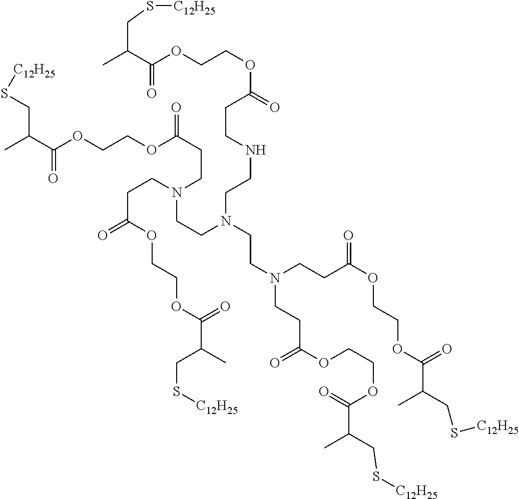 |
| 5A4- 1-SC8 (5- arm) | 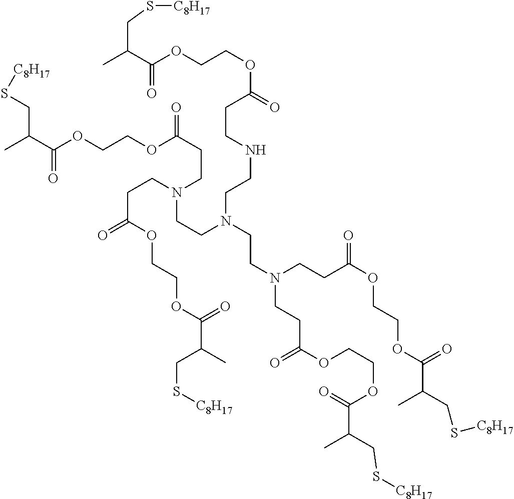 |
| 5A5- SC8 | 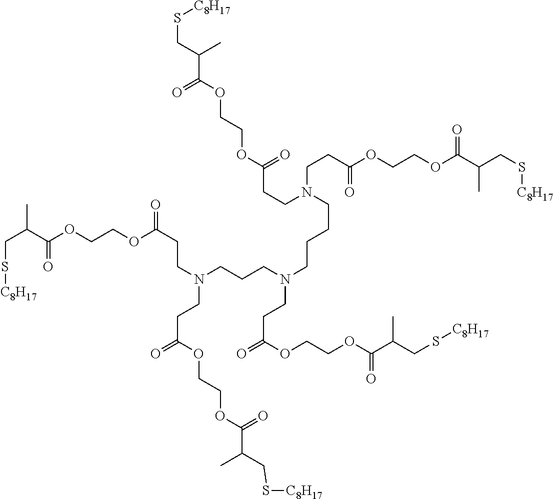 |
| 5A5- SC12 | 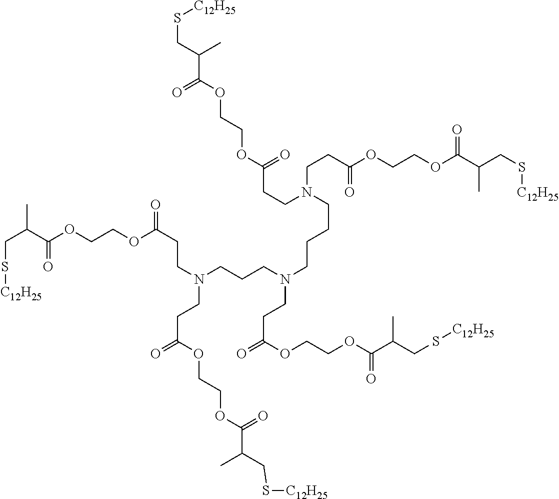 |
| 5A2- 4- SC12 (6- arm) | 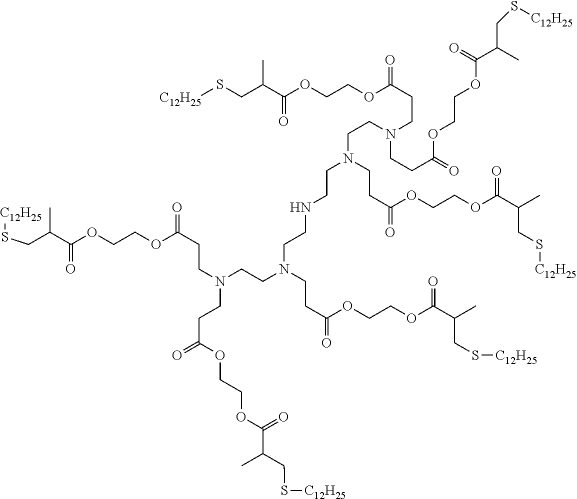 |
| 5A2- 4- SC10 (6- arm) | 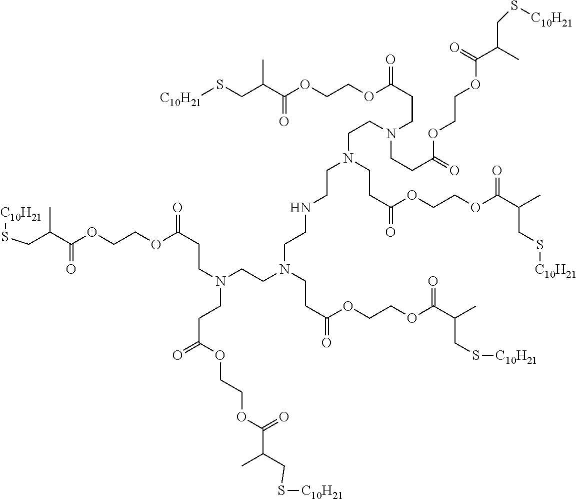 |
| 5A3- 2- SC8 (6- arm) | 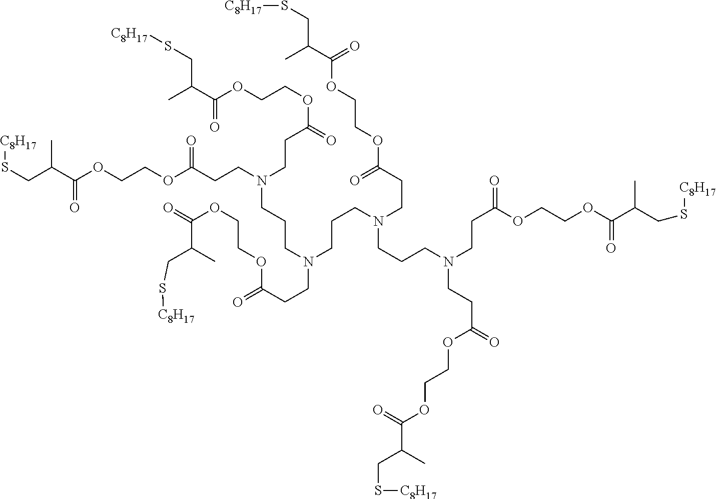 |
| 5A3- 2- SC12 (6- arm) | 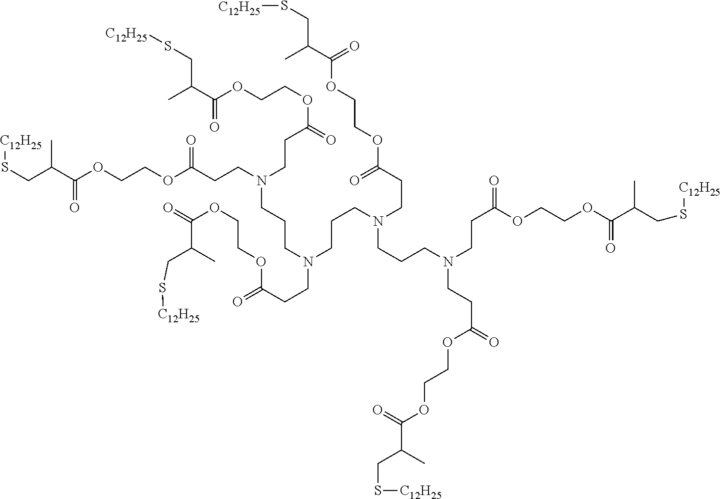 |
| 5A4- 2-SC8 (6- arm) | 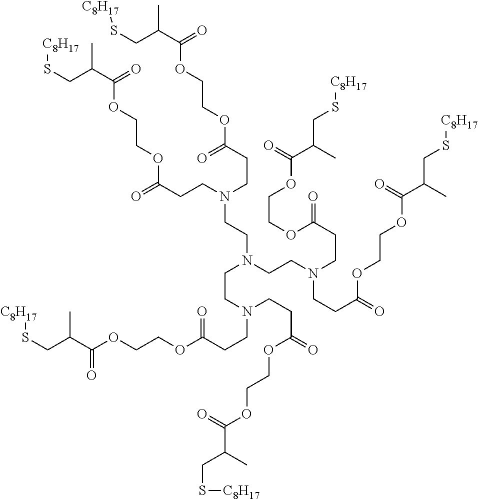 |
| 5A4- 2- SC12 (6- arm) | 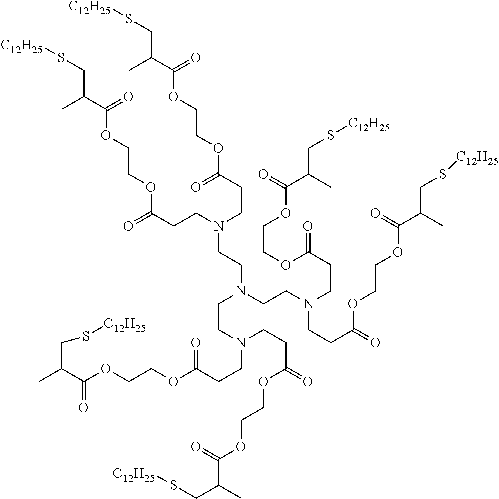 |
| 6A4- SC8 | 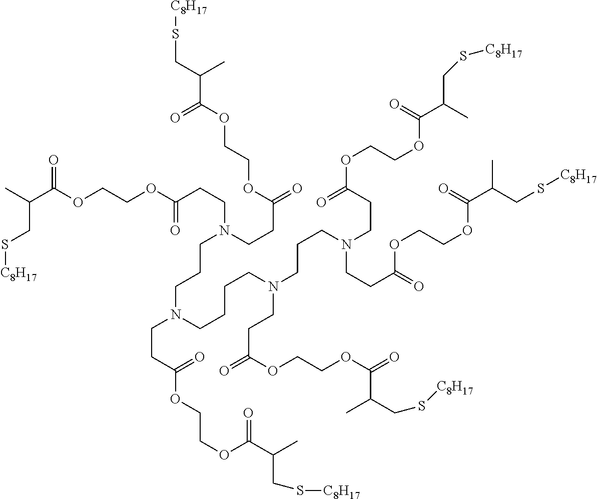 |
| 6A4- SC12 | 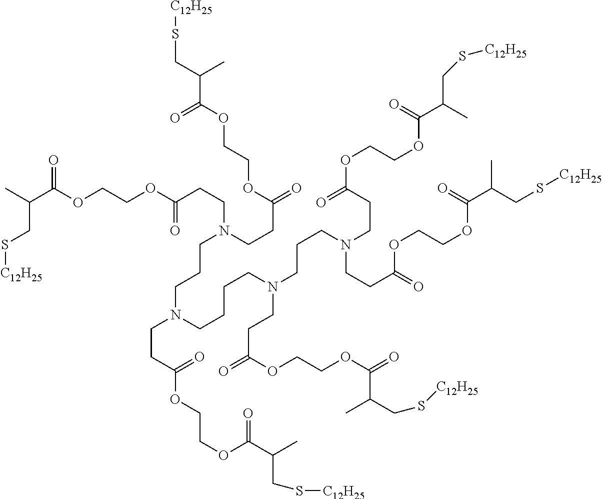 |
| 2A2- g2- SC12 | 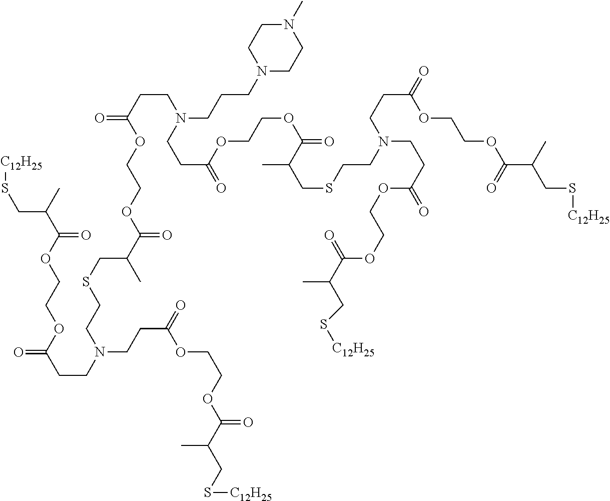 |
| 2A2- g2- SC8 | 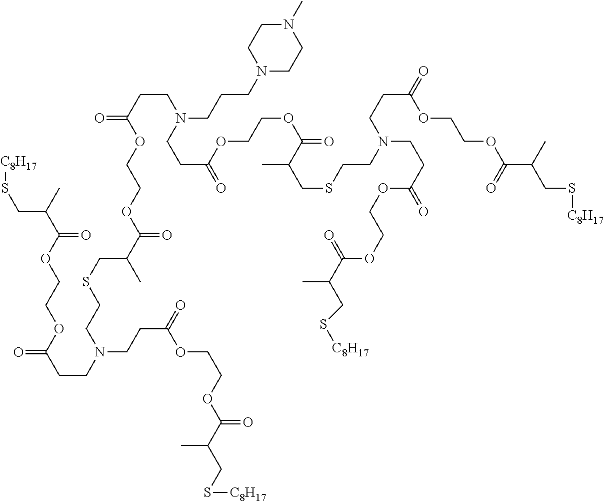 |
| 2A11- g2- SC12 | 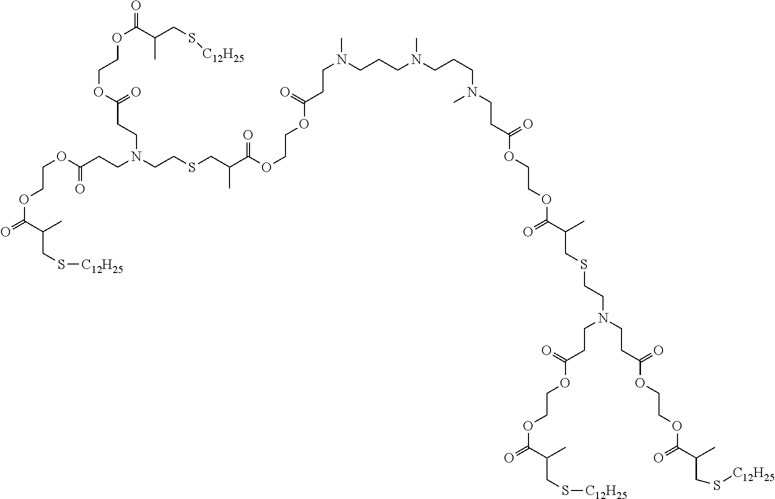 |
| 2A11- g2- SC8 | 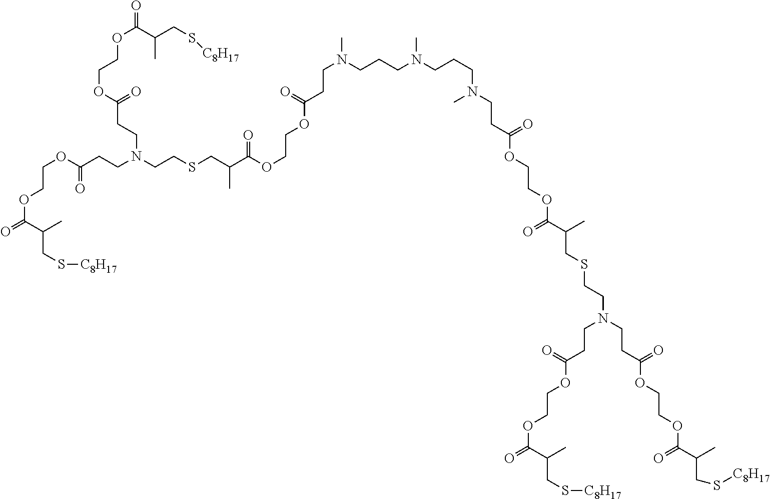 |
| 3A3- g2- SC12 | 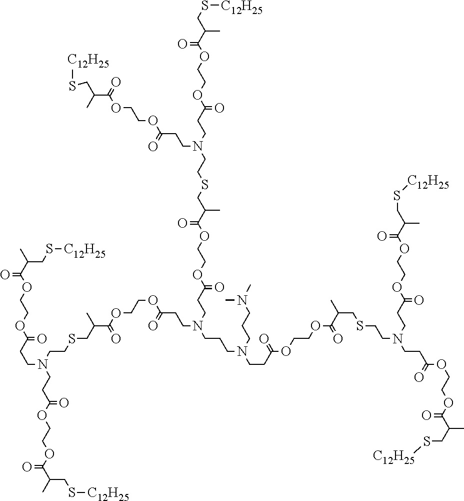 |
| 3A3- g2- SC8 | 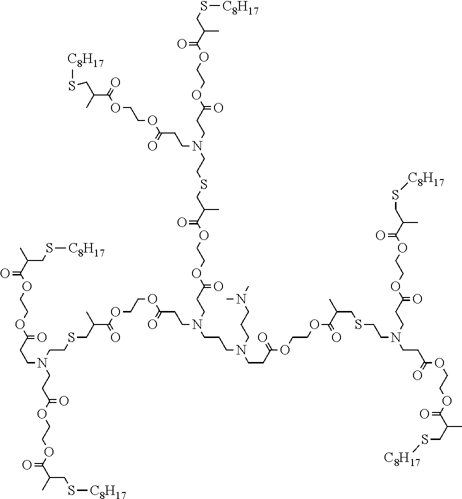 |
| 3A5- g2- SC12 | 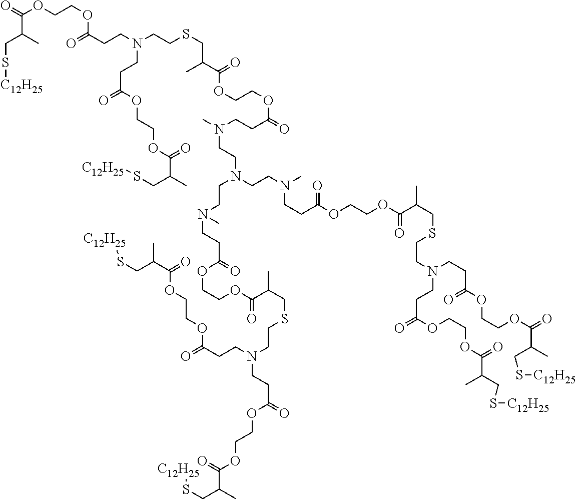 |
| 2A11- g3- SC12 | 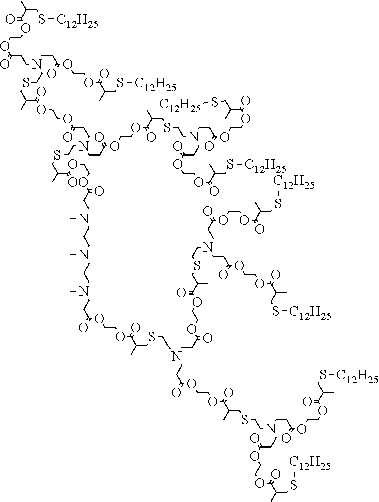 |
| 2A11- g3- SC8 | 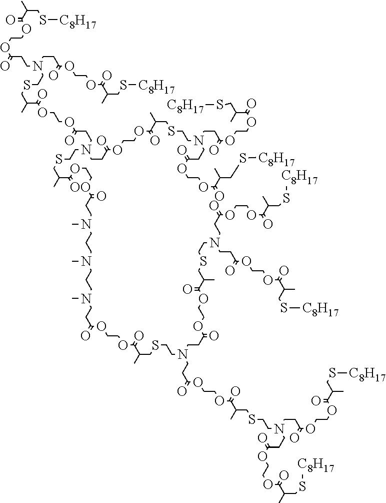 |
| 1A2- g4- SC12 | 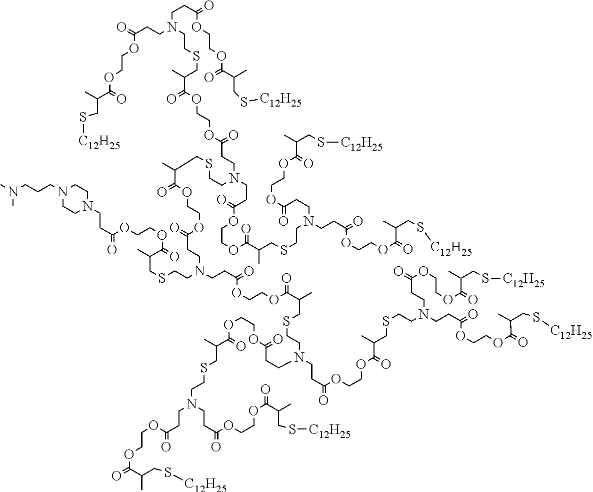 |
| 4A1- g2- SC12 | 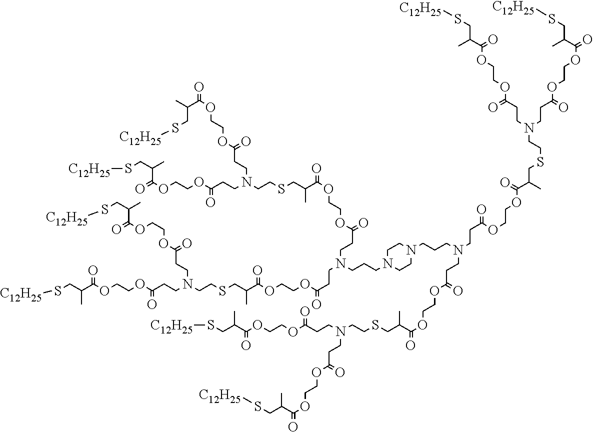 |
| 1A2- g4- SC8 | 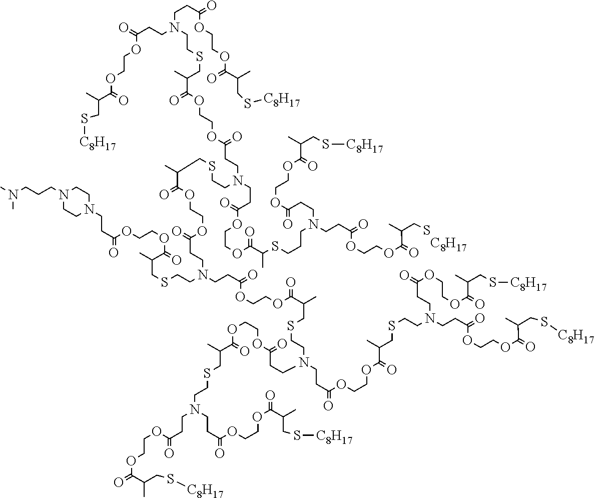 |
| 4A1- g2- SC8 | 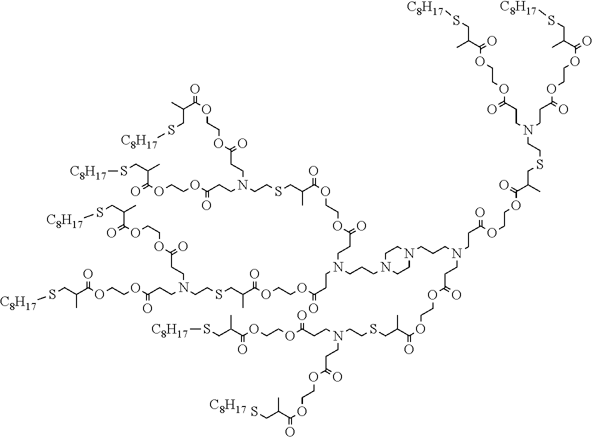 |
| 4A3- g2- SC12 | 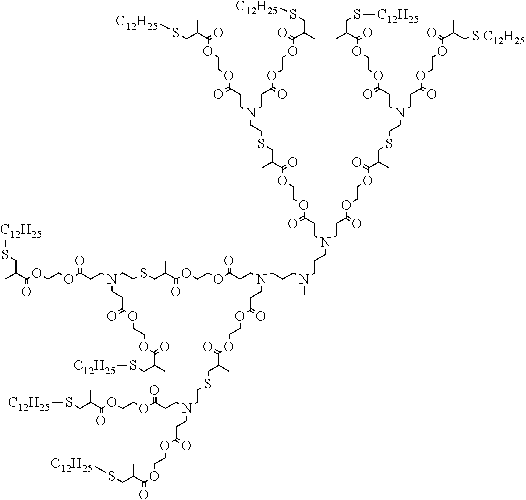 |
| 4A3- g2- SC8 | 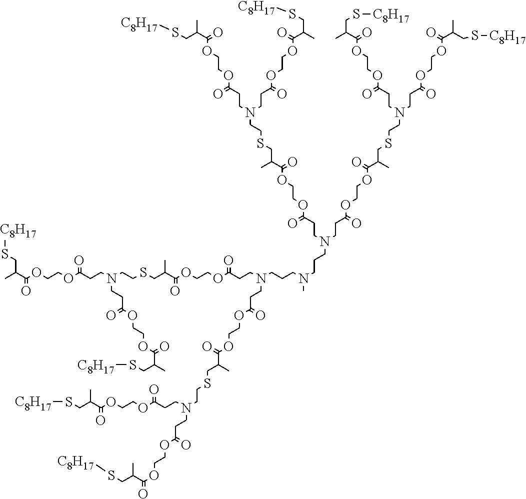 |
| 1A2- g3- SC12 | 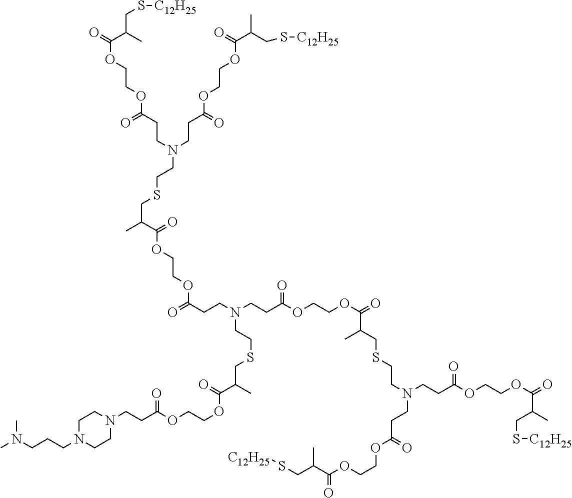 |
| 1A2- g3- SC8 | 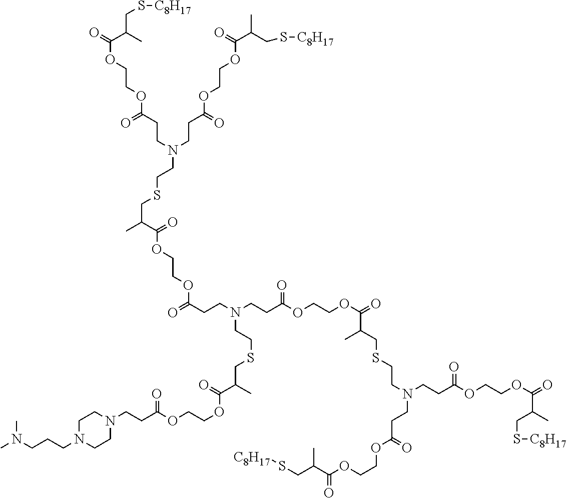 |
| 2A2- g3- SC12 | 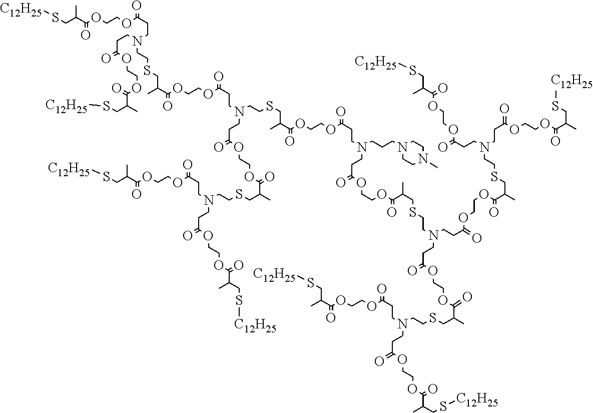 |
| 2A2- g3- SC8 | 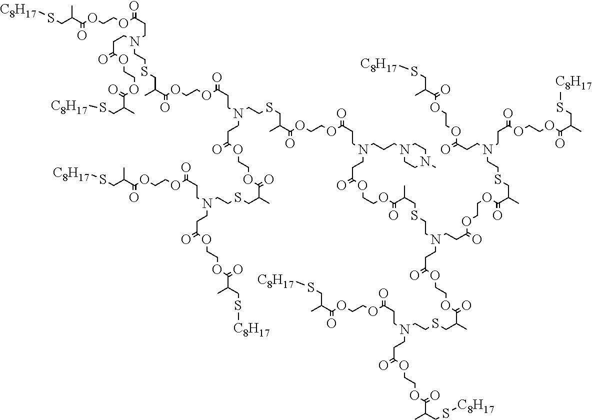 |
| 5A2- 4-SC8 (6- arm) | 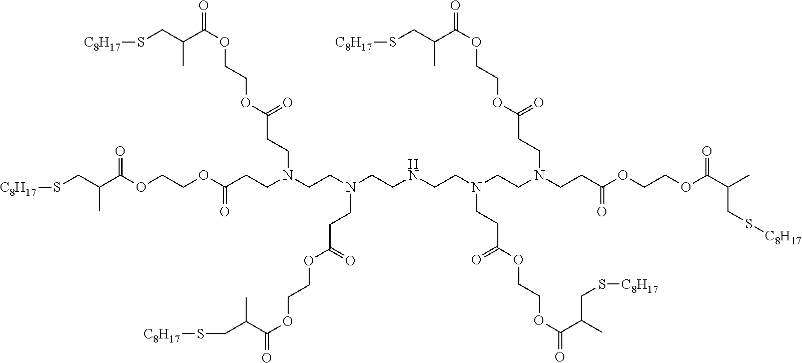 |
| 5A-5- SC8 (6 arm) | 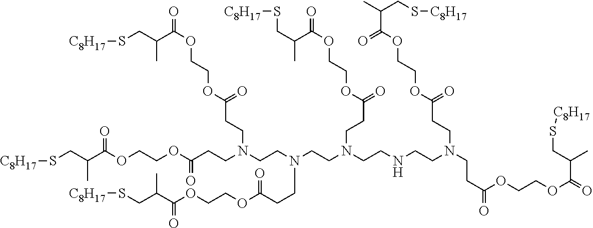 |
| 5A2- 6-SC8 (6 arm) | 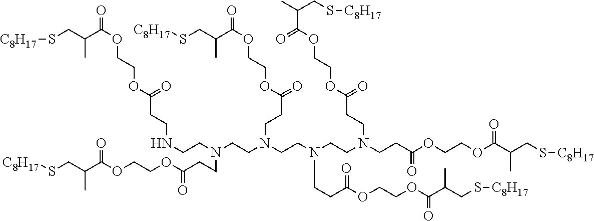 |
| 5A2- 1-SC8 (5- arm) | 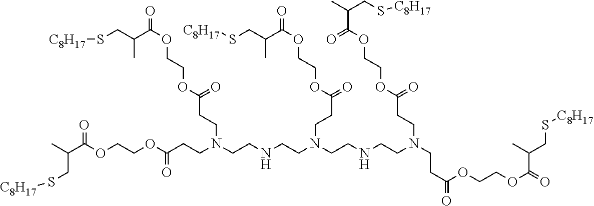 |
| 5A2- 2-SC8 | 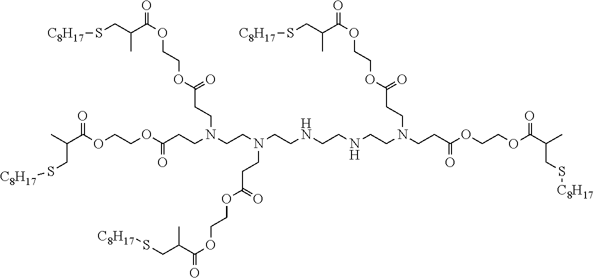 |
| 4A1- SC5 | 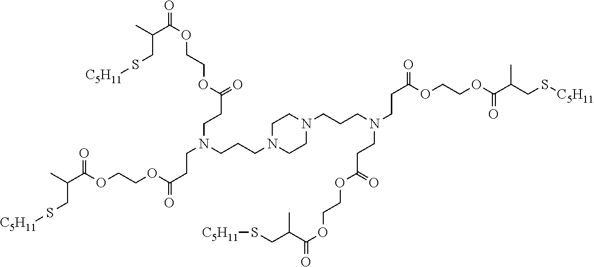 |
| 4A1- SC8 | 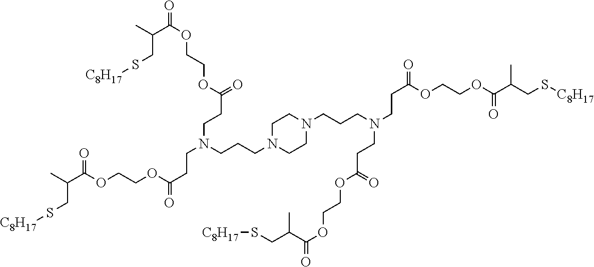 |
| 4A3- SC6 | 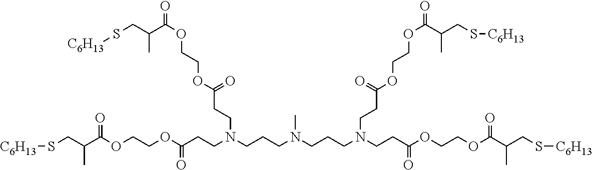 |
| 4A3- SC7 | 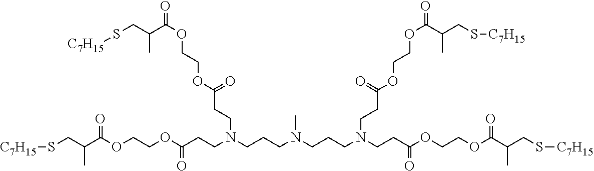 |
| 4A3- SC8 | 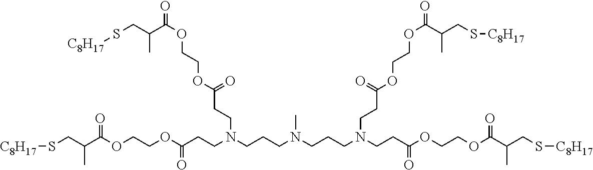 |
| 5A4- 2-SC5 (6 arm) | 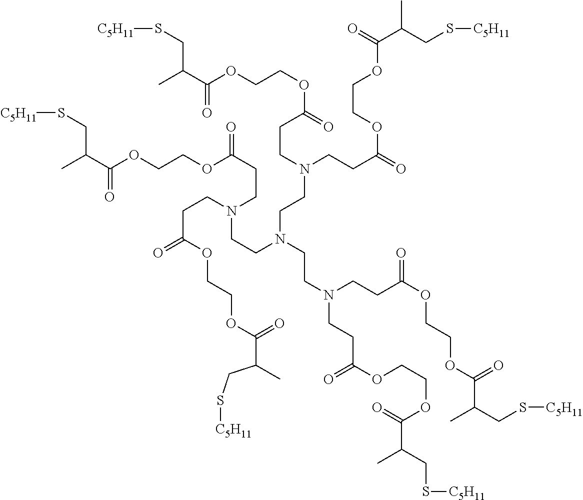 |
| 5A4- 2-SC6 (6 arm) | 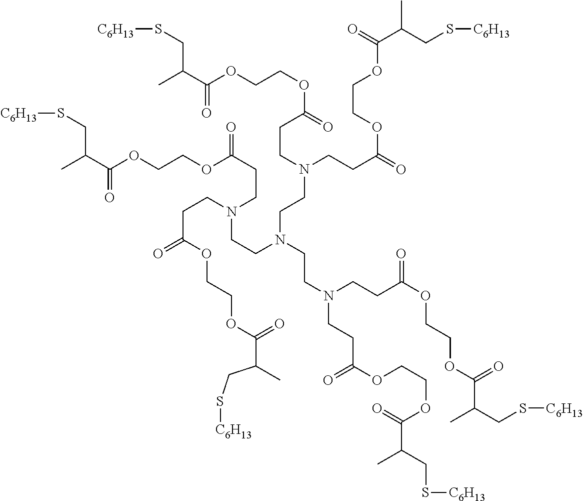 |
| 5A2- 4-SC8 (5- arm) | 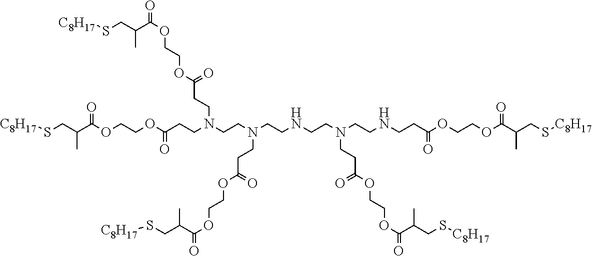 |
| 3A5- g2- SC8 | 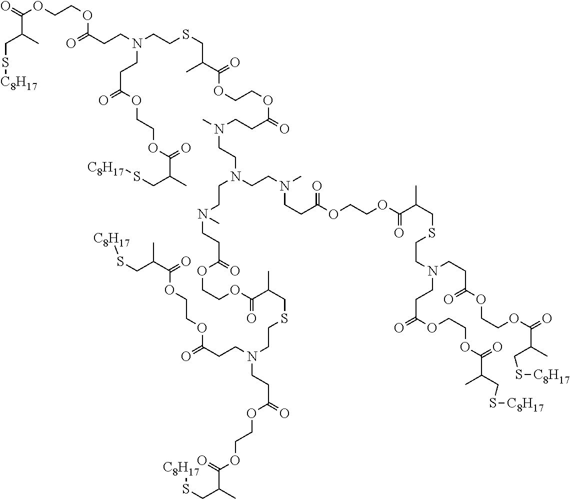 |

C. Modifying the functional groups and/or the chemical properties of the core, repeating units, and the surface or terminating groups, their physical properties can be modulated. Some properties which can be varied include, but are not limited to, solubility, toxicity, immunogenicity and bioattachment capability. Dendrimers are often described by their generation or number of repeating units in the branches. A dendrimer consisting of only the core molecule is referred to as Generation 0, while each consecutive repeating unit along all branches is Generation 1, Generation 2, and so on until the terminating or surface group. In some embodiments, half generations are possible resulting from only the first condensation reaction with the amine and not the second condensation reaction with the thiol.

Preparation of dendrimers requires a level of synthetic control achieved through series of stepwise reactions comprising building the dendrimer by each consecutive group. Dendrimer synthesis can be of the convergent or divergent type. During divergent dendrimer synthesis, the molecule is assembled from the core to the periphery in a stepwise process involving attaching one generation to the previous and then changing functional groups for the next stage of reaction. Functional group transformation is necessary to prevent uncontrolled polymerization. Such polymerization would lead to a highly branched molecule that is not monodisperse and is otherwise known as a hyperbranched polymer. Due to steric effects, continuing to react dendrimer repeat units leads to a sphere shaped or globular molecule, until steric overcrowding prevents complete reaction at a specific generation and destroys the molecule's monodispersity. Thus, in some embodiments, the dendrimers of G1-G10 generation are specifically contemplated. In some embodiments, the dendrimers comprise 1, 2, 3, 4, 5, 6, 7, 8, 9, or 10 repeating units, or any range derivable therein. In some embodiments, the dendrimers used herein are G0, G1, G2, or G3. However, the number of possible generations (such as 11, 12, 13, 14, 15, 20, or 25) may be increased by reducing the spacing units in the branching polymer.

Additionally, dendrimers have two major chemical environments: the environment created by the specific surface groups on the termination generation and the interior of the dendritic structure which due to the higher order structure can be shielded from the bulk media and the surface groups. Because of these different chemical environments, dendrimers have found numerous different potential uses including in therapeutic applications.

In some aspects, the dendrimers that may be used in the present compositions are assembled using the differential reactivity of the acrylate and methacrylate groups with amines and thiols. The dendrimers may include secondary or tertiary amines and thioethers formed by the reaction of an acrylate group with a primary or secondary amine and a methacrylate with a mercapto group. Additionally, the repeating units of the dendrimers may contain groups which are degradable under physiological conditions. In some embodiments, these repeating units may contain one or more germinal diethers, esters, amides, or disulfides groups. In some embodiments, the core molecule is a monoamine which allows dendritic polymerization in only one direction. In other embodiments, the core molecule is a polyamine with multiple different dendritic branches which each may comprise one or more repeating units. The dendrimer may be formed by removing one or more hydrogen atoms from this core. In some embodiments, these hydrogen atoms are on a heteroatom such as a nitrogen atom. In some embodiments, the terminating group is a lipophilic groups such as a long chain alkyl or alkenyl group. In other embodiments, the terminating group is a long chain haloalkyl or haloalkenyl group. In other embodiments, the terminating group is an aliphatic or aromatic group containing an ionizable group such as an amine (—NH₂) or a carboxylic acid (—CO₂H). In still other embodiments, the terminating group is an aliphatic or aromatic group containing one or more hydrogen bond donors such as a hydroxide group, an amide group, or an ester.

In some embodiments, the compositions may further comprise a molar ratio of the ionizable lipids to the total lipid composition from about 15 to about 60. In some embodiments, the molar ratio is from about 10, 15, 20, 25, 30, 35, 40, 45, 50, 55, to about 60 or any range derivable therein. In some embodiments, the molar ratio is from about 30 to about 45.

The cationic ionizable lipids of the present disclosure may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Cationic ionizable lipids may occur as racemates and racemic mixtures, single enantiomers, diastereomeric mixtures and individual diastereomers. In some embodiments, a single diastereomer is obtained. The chiral centers of the cationic ionizable lipids of the present disclosure can have the S or the R configuration. Furthermore, it is contemplated that one or more of the cationic ionizable lipids may be present as constitutional isomers. In some embodiments, the compounds have the same formula but different connectivity to the nitrogen atoms of the core. Without wishing to be bound by any theory, it is believed that such cationic ionizable lipids exist because the starting monomers react first with the primary amines and then statistically with any secondary amines present. Thus, the constitutional isomers may present the fully reacted primary amines and then a mixture of reacted secondary amines.

Chemical formulas used to represent cationic ionizable lipids of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given formula, and regardless of which one is most prevalent, all tautomers of a given chemical formula are intended.

The cationic ionizable lipids of the present disclosure may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the cationic ionizable lipids of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

It should be recognized that the particular anion or cation forming a part of any salt form of a cationic ionizable lipids provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### C. Additional Lipids in the Lipid Nanoparticles

In some aspects of the present disclosure, compositions containing one or more lipids are mixed with the cationic ionizable lipids to create a composition. In some embodiments, the polymers are mixed with 1, 2, 3, 4, or 5 different types of lipids. It is contemplated that the cationic ionizable lipids can be mixed with multiple different lipids of a single type. In some embodiments, the cationic ionizable lipids compositions comprise at least a steroid or a steroid derivative, a PEG lipid, and a phospholipid.

### 1. Steroids and Steroid Derivatives

In some aspects of the present disclosure, the cationic ionizable lipids are mixed with one or more steroid or a steroid derivative to create a composition. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula below:

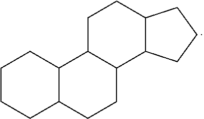 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

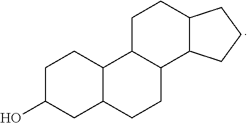 [SMILES: OC1CCC2C(CCC3C4CCCC4CCC23)C1]

In some embodiments of the present disclosure, the steroid or steroid derivative is a cholestane or cholestane derivative. In a cholestane, the ring structure is further defined by the formula:

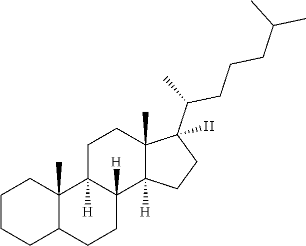

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

In some embodiments, the compositions may further comprise a molar ratio of the steroid to the total lipid composition from about 10 to about 60. In some embodiments, the molar ratio is from about 10, 15, 20, 25, 30, 35, 40, 45, 50, 55, to about 60 or any range derivable therein. In some embodiments, the molar ratio is from about 25 to about 50 such as 30.

### 2. Polymer Conjugated Lipid

In some aspects of the present disclosure, the polymers are mixed with one or more polymer conjugated lipid such as PEGylated lipids (or PEG lipid) to create a dendrimer composition. In some embodiments, the present disclosure comprises using any lipid to which a PEG group has been attached. In some embodiments, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present invention are taught by U.S. Pat. No. 5,820,873, WO 2010/141069, or U.S. Pat. No. 8,450,298, which is incorporated herein by reference.

In another aspect, the PEG lipid has the formula:

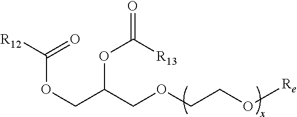

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In another aspect, the PEG lipid has the formula:

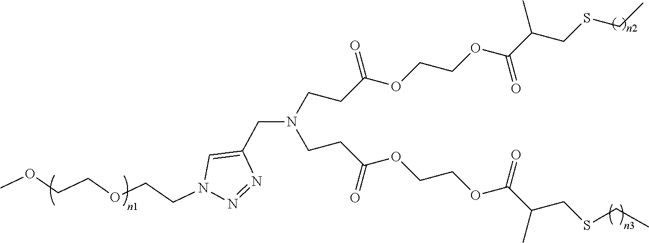

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments, the compositions may further comprise a molar ratio of the PEG lipid to the ionizable total lipid composition from about 0.25, 0.5, 0.75, 1, 1.5, 2, 2.5, 3, 4, 5, 6, 8, 10, 12, to about 12.5 or any range derivable therein. In some embodiments, the molar ratio is from about 1 to about 6.

### 3. Phospholipid

In some aspects of the present disclosure, the polymers are mixed with one or more phospholipids to create a composition. In some embodiments, any lipid which also comprises a phosphate group. In some embodiments, the phospholipid is a structure which contains one or two long chain C₆-C₂₄ alkyl or alkenyl groups, a glycerol or a sphingosine, one or two phosphate groups, and, optionally, a small organic molecule. In some embodiments, the small organic molecule is an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine.

In some embodiments, the compositions may further comprise a molar ratio of the phospholipid to the total lipid composition from about 5 to about 50. In some embodiments, the molar ratio is from about 5, 10, 15, 20, 25, 30, 35, 40, 45, to about 50 or any range derivable therein. In some embodiments, the molar ratio is from about 20 to about 40.

### D. Nucleic Acids and Nucleic Acid Based Therapeutic Agents

### 1. Nucleic Acids

In some aspects of the present disclosure, the dendrimer compositions comprise one or more nucleic acids. In some embodiments, the dendrimer composition comprises one or more nucleic acids present in a weight ratio to the ionizable lipid from about 5:1 to about 1:100. In some embodiments, the weight ratio of nucleic acid to dendrimer is from about 5:1, 2.5:1, 1:1, 1:5, 1:10, 1:15, 1:20, 1:25, 1:30, 1:35, 1:40, 1:45, 1:50, 1:60, 1:70, 1:80, 1:90, or 1:100, or any range derivable therein. In addition, it should be clear that the present disclosure is not limited to the specific nucleic acids disclosed herein. The present invention is not limited in scope to any particular source, sequence, or type of nucleic acid, however, as one of ordinary skill in the art could readily identify related homologs in various other sources of the nucleic acid including nucleic acids from non-human species (e.g., mouse, rat, rabbit, dog, monkey, gibbon, chimp, ape, baboon, cow, pig, horse, sheep, cat and other species). It is contemplated that the nucleic acid used in the present disclosure can comprises a sequence based upon a naturally occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally occurring sequence can encode the same protein as the naturally occurring sequence. In another embodiment, the nucleic acid is a complementary sequence to a naturally occurring sequence, or complementary to at least 80%, 90%, 98%, 98% and 99%. Longer polynucleotides encoding 250, 500, 1000, 1212, 1500, 2000, 2500, 3000 or longer are contemplated herein.

The nucleic acid used herein may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

In some embodiments, the nucleic acid comprises one or more antisense segments which targets a desired HDR site in a gene or gene product. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

In some embodiments, the nucleic acid comprises one or more antisense segments which targets a desired HDR site in a gene or gene product. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to target a gene editing event within a host cell, either in vitro or in vivo, such as within a host animal, including a human subject.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

In some embodiments, the polynucleotide comprising a sequence encoding for a polynucleotide-guided nuclease such as an mRNA comprises from about 250 to about 15,000 nucleotides, from about 500 to about 5,000 nucleotides, from about 800 to about 2,500 nucleotides, or from about 100, 150, 200, 250, 300, 350, 400, 450, 500, 550, 600, 650, 700, 750, 800, 850, 900, 1,000, 2,000, 3,000, 4,000, 5,000, 6,000, 7,000, 8,000, 9,000, 10,000, 11,000, 12,000, 13,000, 14,000, to about 15,000 nucleotides, or any range derivable therein. In some embodiments, the guide polynucleotide, particularly a polynucleotide which has been configured to complex with at least a portion of a target gene or transcript or a polynucleotide with a sequence that encodes for such a guide polynucleotide such as a sgRNA comprises from about 25 to about 500 nucleotides, from about 50 to about 300 nucleotides, from about 80 to about 200 nucleotides or from about 10, 15, 20, 25, 30, 35, 40, 45, 50, 60, 65, 70, 75, 80, 85, 90, 95, 100, 150, 200, 250, 300, 350, 400, 450, to about 500 nucleotides, or any range derivable therein. In some embodiments, the donor polynucleotide, particularly a polynucleotide configured to repair a modified target gene or transcript such as a DNA comprises from about 25 to about 2,500 nucleotides, from about 25 to about 500 nucleotides, from about 50 to about 300 nucleotides, from about 80 to about 200 nucleotides or from about 10, 15, 20, 25, 30, 35, 40, 45, 50, 60, 65, 70, 75, 80, 85, 90, 95, 100, 150, 200, 250, 300, 350, 400, 450, to about 500 nucleotides, or any range derivable therein.

In some embodiments, the composition comprises a weight ratio of the polynucleotide comprising a sequence encoding for a polynucleotide-guided nuclease such as an mRNA to the guide polynucleotide, particularly a polynucleotide which has been configured to complex with at least a portion of a target gene or transcript or a polynucleotide with a sequence that encodes for such a guide polynucleotide such as a sgRNA from about 10:1 to about 1:5, from about 5:1 to about 1:3, from about 3:1 to about 1:2, or from about 10:1, 9:1, 8:1, 7:1, 6:1, 5:1, 4:1, 3:1, 2:1, 1:1, 1:2, 1:3, 1:4, to about 1:5, or any range derivable therein. In some embodiments, the composition comprises a weight ratio of the polynucleotide comprising a sequence encoding for a polynucleotide-guided nuclease such as an mRNA to the donor polynucleotide, particularly a polynucleotide configured to repair a modified target gene or transcript such as a DNA from about 2:1 to about 1:20, from about 1:1 to about 1:10, from about 1:2 to about 1:8, or from about 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, to about 1:20, or any range derivable therein. In some embodiments, the composition comprises a weight ratio of the guide polynucleotide, particularly a polynucleotide which has been configured to complex with at least a portion of a target gene or transcript or a polynucleotide with a sequence that encodes for such a guide polynucleotide such as a sgRNA to the donor polynucleotide, particularly a polynucleotide configured to repair a modified target gene or transcript such as a DNA from about 4:1 to about 1:10, from about 2:1 to about 1:8, from about 1:1 to about 1:4, or from about 4:1, 3:1, 2:1, 2:3, 1:1, 1:2, 1:3, 1:4, 1:5, 1:6, 1:7, 1:8, 1:9, to about 1:10, or any range derivable therein.

In some embodiments, the composition comprises a molar ratio of lipid components to nucleic acid components of from about 1,000:1 to about 5,000:1, from about 2,000:1 to about 4,000:1, or from about 1,000:1, 1,500:1, 2,000:1, 2,500:1, 3,000:1, 3,500:1, 4,000:1, 4,500:1, to about 1,500:1, or any range derivable therein. In some embodiments, the composition comprises an N:P ratio of from about 1:1 to about 20:1, from about 2:1 to about 10:1, from about 4:1 to about 8:1, or from about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, to about 20:1, or any range derivable therein.

### 2. Modified Nucleobases

In some embodiments, the nucleic acids of the present disclosure comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In some embodiments, modified sugar moieties are substituted sugar moieties. In some embodiments, modified sugar moieties are sugar surrogates. Such sugar surrogates may comprise one or more substitutions corresponding to those of substituted sugar moieties.

In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties (see, e.g., PCT International Application WO 2008/101157, for additional 5′,2′-bis substituted sugar moieties and nucleosides).

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃.

In some embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′-CH₂-2′, 4′-(CH₂)₂-2′, 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′, and analogs thereof (see, e.g., U.S. Pat. No. 7,399,845); 4′-C(CH₃)(CH₃)—O-2′ and analogs thereof, (see, e.g., WO 2009/006478); 4′-CH₂—N(OCH₃)-2′ and analogs thereof (see, e.g., WO2008/150729); 4′-CH₂—O—N(CH₃)-2′ (see, e.g., US2004/0171570, published Sep. 2, 2004); 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group (see, U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ and analogs thereof (see, PCT International Application WO 2008/154401).

In some embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA, and (K) Methoxy(ethyleneoxy) (4′-CH(CH₂OMe)-O-2′) BNA (also referred to as constrained MOE or cMOE).

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 5561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US 2004/0171570, US 2007/0287831, and US 2008/0039618; U.S. Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In some embodiments, bicyclic sugar moieties and nucleosides incorporating such bicyclic sugar moieties are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) bicyclic nucleosides have been incorporated into antisense oligonucleotides that showed antisense activity (Frieden et al., Nucleic Acids Research, 2003, 21, 6365-6372).

In some embodiments, substituted sugar moieties comprise one or more non-bridging sugar substituent and one or more bridging sugar substituent (e.g., 5′-substituted and 4′-2′ bridged sugars; PCT International Application WO 2007/134181, wherein LNA is substituted with, for example, a 5′-methyl or a 5′-vinyl group).

In some embodiments, modified sugar moieties are sugar surrogates. In some such embodiments, the oxygen atom of the naturally occurring sugar is substituted, e.g., with a sulfer, carbon or nitrogen atom. In some such embodiments, such modified sugar moiety also comprises bridging and/or non-bridging substituents as described above. For example, certain sugar surrogates comprise a 4′-sulfur atom and a substitution at the 2′-position (see, e.g., published U.S. Patent Application US 2005/0130923) and/or the 5′ position. By way of additional example, carbocyclic bicyclic nucleosides having a 4′-2′ bridge have been described (see, e.g., Freier et al., Nucleic Acids Research, 1997, 25(22), 4429-4443 and Albaek et al., J. Org. Chem., 2006, 71, 7731-7740).

In some embodiments, sugar surrogates comprise rings having other than 5-atoms. For example, in some embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA) (see Leumann, C J. Bioorg. & Med. Chem. (2002) 10:841-854), and fluoro HNA (F-HNA).

In some embodiments, the modified THP nucleosides of Formula VII are provided wherein q₁, q₂, q₃, q₄, q₅, q₆ and q are each H. In certain embodiments, at least one of q₁, q₂, q₃, q₄, q₅, q₆ and q₇ is other than H. In some embodiments, at least one of q₁, q₂, q₃, q₄, q₅, q₆ and q₇ is methyl. In some embodiments, THP nucleosides of Formula VII are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense compounds (see, e.g., review article: Leumann, J. C, Bioorganic & Medicinal Chemistry, 2002, 10, 841-854).

Combinations of modifications are also provided without limitation, such as 2′-F-5′-methyl substituted nucleosides (see PCT International Application WO 2008/101157 for other disclosed 5′,2′-bis substituted nucleosides) and replacement of the ribosyl ring oxygen atom with S and further substitution at the 2′-position (see U.S. Patent Publication US 2005/0130923) or alternatively 5′-substitution of a bicyclic nucleic acid (see PCT International Application WO 2007/134181 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., 2007).

In some embodiments, the present invention provides oligonucleotides comprising modified nucleosides. Those modified nucleotides may include modified sugars, modified nucleobases, and/or modified linkages. The specific modifications are selected such that the resulting oligonucleotides possess desirable characteristics. In some embodiments, oligonucleotides comprise one or more RNA-like nucleosides. In some embodiments, oligonucleotides comprise one or more DNA-like nucleotides.

In some embodiments, nucleosides of the present invention comprise one or more unmodified nucleobases. In certain embodiments, nucleosides of the present invention comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and 0-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (²H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., 1991; and those disclosed by Sanghvi, Y. S., 1993.

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, each of which is herein incorporated by reference in its entirety.

In some embodiments, the present invention provides oligonucleotides comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or (S), α or β such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-5-CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present invention involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety (Letsinger et al., 1989), cholic acid (Manoharan et al., 1994), a thioether, e.g., hexyl-5-tritylthiol (Manoharan et al., 1992; Manoharan et al., 1993), a thiocholesterol (Oberhauser et al., 1992), an aliphatic chain, e.g., dodecandiol or undecyl residues (Saison-Behmoaras et al., 1991; Kabanov et al., 1990; Svinarchuk et al., 1993), a phospholipid, e.g., di-hexadecyl-rac-glycerol or triethylammonium 1,2-di-O-hexadecyl-rac-glycero-3-H-phosphonate (Manoharan et al., 1995; Shea et al., 1990), a polyamine or a polyethylene glycol chain (Manoharan et al., 1995), or adamantane acetic acid (Manoharan et al., 1995), a palmityl moiety (Mishra et al., 1995), or an octadecylamine or hexylamino-carbonyl-oxycholesterol moiety (Crooke et al., 1996).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

### E. Methods of Treatment

Disclosed herein includes methods for treating a subject having or suspected of having a disease or disorder, such as a genetic disease or disorder or a disease or disorder associated with a mutation to one or more genes, the method comprising administering to the subject a composition comprising one or more of each of the following nucleic acids: a polynucleotide comprising a sequence encoding for a polynucleotide-guided nuclease such as an mRNA; a guide polynucleotide, particularly a polynucleotide which has been configured to complex with at least a portion of a target gene or transcript or a polynucleotide with a sequence that encodes for such a guide polynucleotide such as a sgRNA; and the donor polynucleotide, particularly a polynucleotide configured to repair a modified target gene or transcript such as a DNA; and a lipid nanoparticle comprising at least one ionizable lipid; wherein the each of the nucleic acids are encapsulated within the lipid nanoparticle. The subject may be a mammal. The subject may be a non-human species (e.g., mouse, rat, rabbit, dog, monkey, gibbon, chimp, ape, baboon, cow, pig, horse, sheep, cat and other species). The subject may be a human. The subject may be determined to exhibit a mutation in a gene. In some embodiments, the administering comprises systemic (e.g., intravenous) administration. In some embodiments, the subject is selected from the group consisting of mouse, rat, monkey, and human. In some embodiments, the subject is a human.

### F. Methods of Modifying the Genome of a Cell

Some aspects of the disclosure are directed to methods of modifying the genome of a cell in vitro or in vivo in a subject, comprising contacting the cell with a nucleic acid sequence encoding a sequence-targeting nuclease, a guide RNA (e.g., a single guide RNA), and a donor template, wherein the modification comprises the insertion of a nucleotide sequence corresponding to a nucleotide sequence of the donor template (e.g., via homologous recombination with the donor sequence). Homologous recombination (HR) mediated repair (also termed homology-directed repair (HDR)) uses homologous donor DNA as a template to repair a double stranded DNA break. If the sequence of the donor DNA differs from the genomic sequence, this process leads to the introduction of sequence changes into the genome.

The term “modification of the genome” as used herein encompasses the addition of a regulatory sequence or a nucleotide sequence encoding a gene product via homologous recombination (i.e., insertion of a nucleotide sequence corresponding to a nucleotide sequence of the donor template). In some embodiments, the modification comprises replacement of a genomic region associated with a disease or condition (e.g., a genetic mutation) with a non-pathological genomic region via homologous recombination. For example, in some embodiments the modification comprises replacement of a genomic region comprising a mutation with a wild-type or non-mutated genomic region. In some embodiments, the mutation comprises a substitution or deletion mutation. In some embodiments, the modification comprises insertion of a nucleotide sequence in the genome corresponding to a deleted portion of a deletion mutation via homologous recombination. In some embodiments, the modification of the genome comprises insertion and/or replacement of a genomic sequence via homologous recombination that modulates the expression, activity or stability of a gene product. In some embodiments, the modification of the genome comprises modification of both alleles of the cell. In some embodiments, the modification of the genome comprises modification of one allele of the cell.

In some embodiments, the composition results in a homology directed repair rate of at least 1%, at least 5%, at least 15%, at least 25%, at least 50%, or at least 1%, 2%, 3%, 4%, 5%, 6%, 7%, 8%, 9%, 10%, 15%, 20%, 25%, 30%, 35%, 40%, 45%, or at least 50%. In some embodiments, the composition has an indel rate of less than 25%, less than 20%, less than 15%, less than 10%, less than 9%, less than 8%, less than 7%, less than 6%, less than 5%, less than 4%, less than 3%, less than 2%, or less than 1%.

### G. Kits

The present disclosure also provides kits. Any of the components disclosed herein may be combined in the form of a kit. In some embodiments, the kits comprise a composition as described above or in the claims.

The kits will generally include at least one vial, test tube, flask, bottle, syringe or other container, into which a component may be placed, and preferably, suitably aliquoted. Where there is more than one component in the kit, the kit also will generally contain a second, third or other additional containers into which the additional components may be separately placed. However, various combinations of components may be comprised in a container. In some embodiments, all of the lipid nanoparticle components are combined in a single container. In other embodiments, some or all of the lipid nanoparticle components are provided in separate containers.

The kits of the present invention also will typically include packaging for containing the various containers in close confinement for commercial sale. Such packaging may include cardboard or injection or blow molded plastic packaging into which the desired containers are retained. A kit may also include instructions for employing the kit components. Instructions may include variations that can be implemented.

### H. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof, “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, for example, the formula

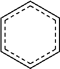

includes

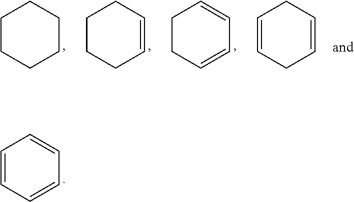

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

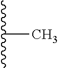

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

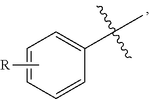

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

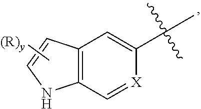

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tert-butyl, t-butyl, t-Bu or ⁱBu), and —CH₂C(CH₃)₃(neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂(cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

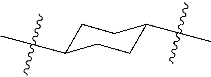 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

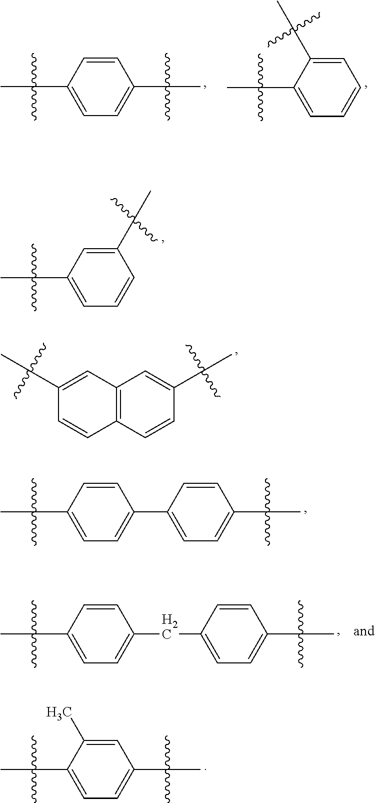

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

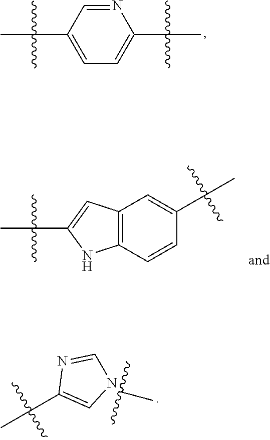

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to an divalent cyclic group, with two carbon atoms, two nitrogen atoms, or one carbon atom and one nitrogen atom as the two points of attachment, said atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

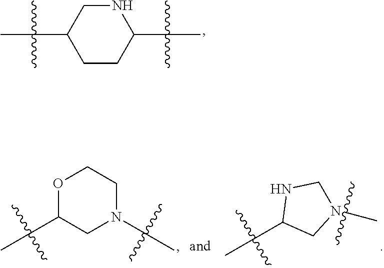

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂(isopropoxy), —OC(CH₃)₃(tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e., an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc. Within the context of the dendrimer, the repeating unit may also be described as the branching unit, interior layers, or generations. Similarly, the terminating group may also be described as the surface group.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### I. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Materials and Methods

### A. Materials

Nucleic acids and other reagents for biological assays. Cas9 messenger RNA (CleanCap Cas9 mRNA-(L-7607)), and Luciferase mRNA were purchased from TriLink BioTechnologies. End modified sgRNA was purchased from Synthego (2′-O-methyl at 3 first and last bases, and 3′ phosphorothioate bonds between first 3 and last 2 bases). DNA oligonucleotides (ssDNA HDR template (Alt-R), sequencing and reference primers, sgRNA primer) were purchased from Integrated DNA Technologies. The Ribogreen reagent was purchased from Life Technologies. One-Glo+Tox was purchased from Promega. CellMask Orange Plasma membrane Stain was purchased from Thermo Fisher Scientific. BFP dest clone (plasmid #71825) was obtained from Addgene and BFP/GFP HEK293 cells were obtained from the laboratory of Professor Jacob Corn (ETH Zurich). Collagenase I, DNAse I, and Hyaluronidase were purchased from Sigma-Aldrich. 10×RBC lysis buffer and cell staining buffer were purchased from Biolegend. QIAquick Gel Extraction Kit and QIAquick PCR Purification Kit were purchased from Qiagen. PureLink Genomic DNA Mini Kit was purchased from Thermo Fisher Scientific. PCR reagents DreamTaq Green PCR Master Mix (2×) and Phusion High-Fidelity PCR Master Mix were purchased from Thermo Fisher Scientific. T7 Endonuclease I was purchased from New England Biolabs. Lipofectamine 2000 and RNAiMAX reagents were purchased from Thermo Fisher Scientific. d-Luciferin Firefly, sodium salt monohydrate was ordered from Goldbio. Ghost Dye Red 780 was purchased from Tonbo Biosciences.

Lipids for dLNPs. Cholesterol was purchased from Sigma Aldrich, 1,2-dioleoyl-sn-glycero-3-phosphoethanolamine (DOPE) was purchased from Avanti Polar Lipids. DMG-PEG2000 was purchased from NOF America Corporation (Sunbright GM-020). All dendrimer lipids were synthesized according to previously reported protocols (Zhou et al., 2016).

Cell culture. Dulbecco's Modified Eagle Medium (DMEM) was purchased from Thermo Fisher Scientific containing high glucose, sodium pyruvate, L-glutamine, and phenol red. RPMI-1640 (ATCC modified) was purchased from Thermo Fisher Scientific and contained L-glutamine, HEPES, phenol red, sodium pyruvate, high glucose, and low sodium bicarbonate. Penicillin-Streptomycin (10,000 U/mL) was purchased from Fisher Scientific. Dulbecco's modified phosphate buffered saline (PBS), Trypsin-EDTA (0.25%) and fetal bovine serum (FBS) were purchased from Sigma-Aldrich. Mutated HEK293 and WT HEK293 cells were cultured in DMEM with 10% FBS and 1% penicillin-streptomycin. IGROV1 cells were cultured in RPMI-1640 with 10% FBS and 1% penicillin-streptomycin.

Animal studies. All experiments were approved by the Institutional Animal Care & Use Committee (IACUC) of The University of Texas Southwestern Medical Center and were consistent with local, state, and federal regulations as applicable. Corning Matrigel Membrane Matrix was purchased from Fisher Scientific. Athymic nude Foxn1^{nu} mice were purchased from Envigo.

### B. Methods

### 1. Instrumentation

Flow cytometry. Flow cytometry for analysis of BFP/GFP signal was performed using BD LSRFortessa machine and HEK293 cell sorting was performed on BD FACSAria™ Fusion machine (BD Biosciences). Flow cytometry data was analyzed using FlowJo v10.6.1.

Confocal laser scanning microscopy. A Zeiss LSM-700 confocal laser scanning microscope was used to imaged treated cells as well as tumor tissue sections. Images were processed using ImageJ (NIH) and Zen 2.6 Blue Edition (Zeiss).

Nanoparticle size and polydispersity. Dynamic light scattering (DLS) was performed to assess nanoparticle size and polydispersity using a Malvern Zetasizer Nano ZS (He—Ne laser, λ=632 nm).

Tissue sectioning. Tumors were sectioned using a Leica CM1900 Cryostat.

Ex vivo animal imaging. All ex vivo imaging was performed using Perkin Elmer IVIS Lumina system and images were processed using Living Image analysis software (Perkin Elmer).

In vitro luminescence and fluorescence assays. Luminescence assays and fluorescence assays were performed using a Tecan Infinite M200 Pro plate reader.

Sanger DNA sequencing. All DNA sequencing was done through the Eugene McDermott Center for Human Growth and Development.

PCR. PCR (Polymerase Chain Reaction) was performed using SimpliAmp Thermocycler from AppliedBiosystems (Thermo Fisher Scientific).

### 2. Nucleic Acid Sequences

Modified sgRNA.
(SEQ ID NO: 1)
5′-GCTGAAGCACTGCACGCCAT-3′
Scrambled sgRNA (SCsgRNA).
(SEQ ID NO: 2)
5′-CAGCATCTTATCTGAGTGGA-3′
DNA sgRNA Recognition Sequence.
(SEQ ID NO: 3)
5′-ATGGCGTGCAGTGCTTCAGC-3′
sgRNA Primer.
(SEQ ID NO: 4)
5′-TAATACGACTCACTATAGGGGCTGAAGCACTGCACGCCATGT
TTTAGAGCTAGAAATAGC-3′
Cas9 mRNA.
(SEQ ID NO: 5)
5′-AUGGCCCCCAAGAAGAAGCGGAAGGUGGGCAUCCACGGCGUG
CCCGCCGCCGACAAGAAGUACAGCAUCGGCCUGGACAUCGGCACC
AACAGCGUGGGCUGGGCCGUGAUCACCGACGAGUACAAGGUGCCC
AGCAAGAAGUUCAAGGUGCUGGGCAACACCGACCGGCACAGCAUC
AAGAAGAACCUGAUCGGCGCCCUGCUGUUCGACAGCGGCGAGACC
GCCGAGGCCACCCGGCUGAAGCGGACCGCCCGGCGGCGGUACACC
CGGCGGAAGAACCGGAUCUGCUACCUGCAGGAGAUCUUCAGCAAC
GAGAUGGCCAAGGUGGACGACAGCUUCUUCCACCGGCUGGAGGAG
AGCUUCCUGGUGGAGGAGGACAAGAAGCACGAGCGGCACCCCAUC
UUCGGCAACAUCGUGGACGAGGUGGCCUACCACGAGAAGUACCCC
ACCAUCUACCACCUGCGGAAGAAGCUGGUGGACAGCACCGACAAG
GCCGACCUGCGGCUGAUCUACCUGGCCCUGGCCCACAUGAUCAAG
UUCCGGGGCCACUUCCUGAUCGAGGGCGACCUGAACCCCGACAAC
AGCGACGUGGACAAGCUGUUCAUCCAGCUGGUGCAGACCUACAAC
CAGCUGUUCGAGGAGAACCCCAUCAACGCCAGCGGCGUGGACGCC
AAGGCCAUCCUGAGCGCCCGGCUGAGCAAGAGCCGGCGGCUGGAG
AACCUGAUCGCCCAGCUGCCCGGCGAGAAGAAGAACGGCCUGUUC
GGCAACCUGAUCGCCCUGAGCCUGGGCCUGACCCCCAACUUCAAG
AGCAACUUCGACCUGGCCGAGGACGCCAAGCUGCAGCUGAGCAAG
GACACCUACGACGACGACCUGGACAACCUGCUGGCCCAGAUCGGC
GACCAGUACGCCGACCUGUUCCUGGCCGCCAAGAACCUGAGCGAC
GCCAUCCUGCUGAGCGACAUCCUGCGGGUGAACACCGAGAUCACC
AAGGCCCCCCUGAGCGCCAGCAUGAUCAAGCGGUACGACGAGCAC
CACCAGGACCUGACCCUGCUGAAGGCCCUGGUGCGGCAGCAGCUG
CCCGAGAAGUACAAGGAGAUCUUCUUCGACCAGAGCAAGAACGGC
UACGCCGGCUACAUCGACGGCGGCGCCAGCCAGGAGGAGUUCUAC
AAGUUCAUCAAGCCCAUCCUGGAGAAGAUGGACGGCACCGAGGAG
CUGCUGGUGAAGCUGAACCGGGAGGACCUGCUGCGGAAGCAGCGG
ACCUUCGACAACGGCAGCAUCCCCCACCAGAUCCACCUGGGCGAG
CUGCACGCCAUCCUGCGGCGGCAGGAGGACUUCUACCCCUUCCUG
AAGGACAACCGGGAGAAGAUCGAGAAGAUCCUGACCUUCCGGAUC
CCCUACUACGUGGGCCCCCUGGCCCGGGGCAACAGCCGGUUCGCC
UGGAUGACCCGGAAGAGCGAGGAGACCAUCACCCCCUGGAACUUC
GAGGAGGUGGUGGACAAGGGCGCCAGCGCCCAGAGCUUCAUCGAG
CGGAUGACCAACUUCGACAAGAACCUGCCCAACGAGAAGGUGCUG
CCCAAGCACAGCCUGCUGUACGAGUACUUCACCGUGUACAACGAG
CUGACCAAGGUGAAGUACGUGACCGAGGGCAUGCGGAAGCCCGCC
UUCCUGAGCGGCGAGCAGAAGAAGGCCAUCGUGGACCUGCUGUUC
AAGACCAACCGGAAGGUGACCGUGAAGCAGCUGAAGGAGGACUAC
UUCAAGAAGAUCGAGUGCUUCGACAGCGUGGAGAUCAGCGGCGUG
GAGGACCGGUUCAACGCCAGCCUGGGCACCUACCACGACCUGCUG
AAGAUCAUCAAGGACAAGGACUUCCUGGACAACGAGGAGAACGAG
GACAUCCUGGAGGACAUCGUGCUGACCCUGACCCUGUUCGAGGAC
CGGGAGAUGAUCGAGGAGCGGCUGAAGACCUACGCCCACCUGUUC
GACGACAAGGUGAUGAAGCAGCUGAAGCGGCGGCGGUACACCGGC
UGGGGCCGGCUGAGCCGGAAGCUGAUCAACGGCAUCCGGGACAAG
CAGAGCGGCAAGACCAUCCUGGACUUCCUGAAGAGCGACGGCUUC
GCCAACCGGAACUUCAUGCAGCUGAUCCACGACGACAGCCUGACC
UUCAAGGAGGACAUCCAGAAGGCCCAGGUGAGCGGCCAGGGCGAC
AGCCUGCACGAGCACAUCGCCAACCUGGCCGGCAGCCCCGCCAUC
AAGAAGGGCAUCCUGCAGACCGUGAAGGUGGUGGACGAGCUGGUG
AAGGUGAUGGGCCGGCACAAGCCCGAGAACAUCGUGAUCGAGAUG
GCCCGGGAGAACCAGACCACCCAGAAGGGCCAGAAGAACAGCCGG
GAGCGGAUGAAGCGGAUCGAGGAGGGCAUCAAGGAGCUGGGCAGC
CAGAUCCUGAAGGAGCACCCCGUGGAGAACACCCAGCUGCAGAAC
GAGAAGCUGUACCUGUACUACCUGCAGAACGGCCGGGACAUGUAC
GUGGACCAGGAGCUGGACAUCAACCGGCUGAGCGACUACGACGUG
GACCACAUCGUGCCCCAGAGCUUCCUGAAGGACGACAGCAUCGAC
AACAAGGUGCUGACCCGGAGCGACAAGAACCGGGGCAAGAGCGAC
AACGUGCCCAGCGAGGAGGUGGUGAAGAAGAUGAAGAACUACUGG
CGGCAGCUGCUGAACGCCAAGCUGAUCACCCAGCGGAAGUUCGAC
AACCUGACCAAGGCCGAGCGGGGCGGCCUGAGCGAGCUGGACAAG
GCCGGCUUCAUCAAGCGGCAGCUGGUGGAGACCCGGCAGAUCACC
AAGCACGUGGCCCAGAUCCUGGACAGCCGGAUGAACACCAAGUAC
GACGAGAACGACAAGCUGAUCCGGGAGGUGAAGGUGAUCACCCUG
AAGAGCAAGCUGGUGAGCGACUUCCGGAAGGACUUCCAGUUCUAC
AAGGUGCGGGAGAUCAACAACUACCACCACGCCCACGACGCCUAC
CUGAACGCCGUGGUGGGCACCGCCCUGAUCAAGAAGUACCCCAAG
CUGGAGAGCGAGUUCGUGUACGGCGACUACAAGGUGUACGACGUG
CGGAAGAUGAUCGCCAAGAGCGAGCAGGAGAUCGGCAAGGCCACC
GCCAAGUACUUCUUCUACAGCAACAUCAUGAACUUCUUCAAGACC
GAGAUCACCCUGGCCAACGGCGAGAUCCGGAAGCGGCCCCUGAUC
GAGACCAACGGCGAGACCGGCGAGAUCGUGUGGGACAAGGGCCGG
GACUUCGCCACCGUGCGGAAGGUGCUGAGCAUGCCCCAGGUGAAC
AUCGUGAAGAAGACCGAGGUGCAGACCGGCGGCUUCAGCAAGGAG
AGCAUCCUGCCCAAGCGGAACAGCGACAAGCUGAUCGCCCGGAAG
AAGGACUGGGACCCCAAGAAGUACGGCGGCUUCGACAGCCCCACC
GUGGCCUACAGCGUGCUGGUGGUGGCCAAGGUGGAGAAGGGCAAG
AGCAAGAAGCUGAAGAGCGUGAAGGAGCUGCUGGGCAUCACCAUC
AUGGAGCGGAGCAGCUUCGAGAAGAACCCCAUCGACUUCCUGGAG
GCCAAGGGCUACAAGGAGGUGAAGAAGGACCUGAUCAUCAAGCUG
CCCAAGUACAGCCUGUUCGAGCUGGAGAACGGCCGGAAGCGGAUG
CUGGCCAGCGCCGGCGAGCUGCAGAAGGGCAACGAGCUGGCCCUG
CCCAGCAAGUACGUGAACUUCCUGUACCUGGCCAGCCACUACGAG
AAGCUGAAGGGCAGCCCCGAGGACAACGAGCAGAAGCAGCUGUUC
GUGGAGCAGCACAAGCACUACCUGGACGAGAUCAUCGAGCAGAUC
AGCGAGUUCAGCAAGCGGGUGAUCCUGGCCGACGCCAACCUGGAC
AAGGUGCUGAGCGCCUACAACAAGCACCGGGACAAGCCCAUCCGG
GAGCAGGCCGAGAACAUCAUCCACCUGUUCACCCUGACCAACCUG
GGCGCCCCCGCCGCCUUCAAGUACUUCGACACCACCAUCGACCGG
AAGCGGUACACCAGCACCAAGGAGGUGCUGGACGCCACCCUGAUC
CACCAGAGCAUCACCGGCCUGUACGAGACCCGGAUCGACCUGAGC
CAGCUGGGCGGCGACAGCGGCGGCAAGCGGCCCGCCGCCACCAAG
AAGGCCGGCCAGGCCAAGAAGAAGAAGGGCAGCUACCCCUACGAC
GUGCCCGACUACGCCUGA-3′
Scrambled ssDNA.
(SEQ ID NO: 6)
5′-CCAGGCCTCTGATTCCTCACTGATTGCTCTTAGGTCTGGCCC
CTCCTCAGCATCTTATCCGCGTTGAAGGAAATTTGCGTGTGGAGT
ATTTGGATGACAGAAACACTTTTCGGCATAGTG-3′
SSDNA HDR Template.
(SEQ ID NO: 7)
5′-GCCACCTACGGCAAGCTGACCCTGAAGTTCATCTGCACCACC
GGCAAGCTGCCCGTGCCCTGGCCCACCCTCGTGACCACCCTGACG
TACGGCGTGCAGTGCTTCAGCCGCTACCCCGACCACATGA-3′
Sequencing Primers.
B/GFP Fwd 1
(SEQ ID NO: 8)
5′-AGCTGGCTAGGTAAGCTTGG-3′
B/GFP Fwd 2
(SEQ ID NO: 9)
5′-TGGGTGGAGACTGAAGTTAGGC-3′
B/GFP Rev 1
(SEQ ID NO: 10)
5′-CTTGTACAGCTCGTCCATGC-3′
B/GFP Rev 2
(SEQ ID NO: 11)
5′-GGGTGCTCAGGTAGTGGTT-3′
Reference Sequence Primers (TIDER)
B/GFP Ref Fwd
(SEQ ID NO: 12)
5′-CCCTGACGTACGGCGTG-3′
B/GFP Ref Rev
(SEQ ID NO: 13)
5′-CACGCCGTACGTCAGGG-3′

### 3. Experimental Details

dLNP component mole ratio screen. dLNPs were prepared via the ethanol dilution method using a Box-Behnken experimental design with five different variables for analysis: Mole ratio of dendrimer:nucleic acid (2000, 4000, 6000); intra-particle dendrimer (30, 50, 70); cholesterol (20, 40, 60); DOPE (20, 40, 60); DMG-PEG2K (0.5, 1.5, 2.5). IGROV1 cells were transfected with the dLNPs and luminescence values were determined as described in the luciferase mRNA delivery assay protocol listed below.

In vitro dLNP formulation. dLNPs were prepared via the ethanol dilution method. Luciferase mRNA was diluted in acidic aqueous buffer (0.01M citric acid/sodium citrate buffer, pH 3). Stock solutions of each lipid component at specific molar concentrations were created via dilution in ethanol. These stock solutions were then combined together at the molar ratio of 38.5:30:30:1.5 (dendrimer:cholesterol:DOPE:DMG-PEG). This lipid mixture was added to the mRNA solution at a volumetric ratio of 1:3 (lipid mixture:mRNA solution) and mixed rapidly using a micropipette. Following mixing, the dLNPs were allowed to incubate at room temperature (RT) for 15-20 minutes and then either diluted in (by volume, 3×) or dialyzed against 1× Dulbecco's Modified PBS without calcium and magnesium (Sigma-Aldrich). If dialyzed, a 2 L solution of 1×PBS was created via dilution of 10×PBS (Sigma-Aldrich) in deionized and autoclaved H₂O and dLNPs were loaded into Pur-A-Lyzer Midi dialysis chambers (Sigma-Aldrich). The loaded dLNPs were then dialyzed in the 1×PBS for a duration of 1 hour per 200 μL of sample in the chamber.

Nucleic acid binding experiments. Nucleic acid binding was determined using the Quant-iT Ribogreen assay (Fisher Scientific). dLNPs were first prepared using the in vitro or in vivo dLNP preparation method. These dLNPs were added to a 96-well black opaque polystyrene microplate (Corning-Fisher Scientific) at a volume corresponding to the treatment dosage. A standard curve of the appropriate nucleic acid was prepared in the same medium as the dLNPs for consistency. The Ribogreen reagent was diluted 1000-fold in 1×PBS and added to each well at a volume of 50 μL using a multichannel micropipette. Using an orbital shaker, the dLNPs and Ribogreen reagent mixture were shaken for 5 minutes at RT. Each well was then measured for fluorescence (λ_{Ex} 485 nm, λ_{Em} 535 nm) and the amount of free mRNA was assessed once fitted to the standard curve. Free mRNA was used to determine encapsulated mRNA percentage via the following formula: Fraction of bound nucleic acid=[(total nucleic acid added−free nucleic acid)/(total nucleic acid added)] (N=4+/−standard deviation in all cases).

dLNP physical property characterization. dLNP properties were determined using a Zetasizer Nano ZS (Malvern) with a helium-neon laser (λ=632 nm). Size and polydispersity of the dLNPs were measured using dynamic light scattering (DLS) by 1730 back scattering with the following settings: 5 measurements, 3 runs at 10 seconds per run, and attenuation set to automatic. The refraction index was adjusted appropriately for each sample to account for any possible viscosity differences between dLNP formulations as indicated via Malvern guidance.

Luciferase mRNA delivery assay. dLNPs were made with Luciferase mRNA (Tri-Link Biotechnologies) using the ethanol dilution method described above. IGROV1 cells (for molar ratio optimization, other cell lines were used for the dendrimer library screen as described above) were seeded in a white opaque 96 well microplate (Corning-Fisher Scientific) at a density of 4×10³ cells per well in 100 μL of RPMI 1640 medium containing 10% FBS and 1% penicillin/streptomycin and allowed to adhere to the well overnight at 37° C. Following overnight incubation, the dLNPs were prepared containing Luciferase mRNA and added to each well using a multichannel micropipette at a dose of 50 ng of mRNA/well. 24 hours after the addition of dLNPs, 100 μL of additional RPMI 1640 containing 10% FBS and 1% penicillin/streptomycin was then added to each well via a multichannel micropipette. The cells were then incubated at 37° C. for 24 additional hours resulting in a total transfection time of 48 hours. After the 48-hour incubation period, cell viability and luciferase expression were assessed using the ONE-Glo+Tox Assay (Promega) according to the manufacturer protocol and normalized to viability (N=4+/−standard deviation).

Dendrimer library screen. The dendrimer library analyzed consisted of the amine cores 3A3, 3A5, 4A3, and 4A1, as well as the alkyl periphery groups SC5, SC6, SC7, SC8, SC9, SC10, SC11, SC12, and SC14; totaling 36 distinct dendrimer compounds. Each of these compounds were used to transfect three cell lines (IGROV1, HEK293T, and HeLa). All cells were seeded in white opaque 96 well microplates at different densities in 100 μL of their respective media (IGROV1: 4×10³ cells/well—RPMI1640 10% FBS 1% penicillin/streptomycin, HEK293T 1×10⁴ cells/well—DMEM 10% FBS 1% penicillin/streptomycin, HeLa: 4×10³ cells/well—DMEM 10% FBS 1% penicillin/streptomycin) and incubated at 37° C. overnight so that they could adhere to the wells. Each of the dendrimer lipids were combined with ethanol to form a 10 mM working stock solution. These stock solutions were then combined with cholesterol, DOPE, and DMG-PEG at a molar ratio of 38.5:30:30:1.5 (dendrimer:cholesterol:DOPE:DMG-PEG), wherein each dendrimer was fixed at a molar ratio of 10,000:1 (dendrimer:mRNA). Each dLNP mixture was then combined with luciferase mRNA in acidic aqueous buffer (as previously described) at a ratio of 1:3 (lipid mixture in ethanol:mRNA in acidic aqueous buffer) by volume and rapidly mixed using a micropipette. Once mixed, the dLNPs were allowed to incubate at RT for 15 minutes and then diluted threefold by volume in 1×PBS. After overnight incubation, all cells were transfected with the following dLNPs containing 12.5 ng luciferase mRNA and then 100 μL of additional media was added to each well using a multichannel micropipette. Cells were incubated for 48 hours at 37° C. following transfection and then analyzed using a microplate reader for luciferase expression and toxicity. Luciferase expression and toxicity were evaluated using the ONE-Glo+Tox Assay (Promega) according to the manufacturer protocol and normalized to background and viability (N=4+/−standard deviation).

Dose response. Selected dendrimer compounds with the 4A3 amine core and alkyl peripheries SC5, SC6, SC7, SC8, SC9, SC10, SC11, SC12, and SC14 were examined in a dose-response fashion for efficacy and toxicity. dLNPs containing luciferase mRNA were created via the ethanol dilution method (molar ratio of 38.5:30:30:1.5, dendrimer:cholesterol:DOPE:DMG-PEG) and administered to three different cell lines (IGROV1, HEK293T, HeLa) in the same fashion as the dendrimer library screen, but at 4 different doses (6.25 ng, 12.5 ng, 25 ng, 50 ng). Luciferase expression and toxicity were determined via the ONE-Glo+Tox Assay (Promega) (N=3+/−standard deviation).

sgRNA sequence validation. The sgRNA sequence was validated using an in vitro cutting assay. Briefly, 300 ng of linearized pDNA (BFP dest clone, addgene) was mixed with 160 ng of Cas9 protein and 33 ng of IVT sgRNA RNPs in 1×NE buffer 3.1 (final volume: 20 μL). Then, the mixture was incubated at 37° C. for 2 hours. The cleavage was detected by agarose electrophoresis (2% agarose gel).

dLNP multi-encapsulation of HDR components. HEK293 B/GFP cells were plated in a 12 well tissue culture dish (CytoOne) at a density of 1.5×10⁵ cells per well in 500 μL of DMEM containing 10% FBS and 1% penicillin/streptomycin and allowed to attach overnight at 37° C. After overnight incubation, 4A3-SC8 dLNPs (38.5:30:30:1.5; 4A3-SC8:cholesterol:DOPE:DMG-PEG) were made using the ethanol dilution method, however multiple components were added into the nucleic acid mixture for encapsulation. In the first round of evaluation, a two particle, sequential delivery system was implemented with the first dLNPs containing only Cas9 mRNA (500 ng) and the other dLNPs containing one of three different cargoes: modified sgRNA only (250 ng), both ssDNA HDR template and modified sgRNA (fixed at a 1:1 ratio by weight, 250 ng each), or both ssDNA HDR template and modified sgRNA (fixed at a 1:1 ratio by moles, 500 ng total nucleic acid). The dLNPs containing Cas9 mRNA were administered to the cells at a dose of 500 ng per well following overnight incubation. 24 hours after transfection with dLNPs containing Cas9 mRNA, the second dLNPs were administered to the cells containing one of three different cargoes and cells were incubated at 37° C. for 48 hours. 24 hours after transfection with the second round of dLNPs, 1 mL of DMEM was added to each well. Following the 48-hour incubation, cells were prepared for analysis via flow cytometry. Briefly, all media in each well was aspirated and each well was then rinsed with 1 mL of 1×PBS. 1×PBS was aspirated and 500 μL of Trypsin-EDTA was added to each well and the cells were incubated at 37° C. for 2 minutes. After incubation, 1 mL of DMEM was added to each well and the contents of all wells were collected in 1.5 mL tubes (Eppendorf) and spun at 300 G for 4 minutes to generate a cell pellet. The cell pellet was then resuspended in 1 mL of 1×PBS and placed on ice until analyzed via flow cytometry. In the second round of evaluation, a simultaneous delivery system consisting of a three, two, or a single particle was evaluated. HEK293 B/GFP cells were seeded in a 12 well plate using the same method as described above. In the three-particle system, 4A3-SC8 dLNPs were created using the ethanol dilution method and contained a single nucleic cargo each (500 ng Cas9 mRNA, 250 ng modified sgRNA, 250 ng ssDNA HDR template). In the two-particle system, one set of 4A3-SC8 dLNPs were created containing 500 ng of Cas9 mRNA, and the other set of 4A3-SC8 dLNPs contained a 1:1 ratio of ssDNA HDR Template and modified sgRNA (500 ng total nucleic acid). Finally, in the single particle system, 4A3-SC8 dLNPs were created that contained all three nucleic acid cargoes fixed by weight at a ratio of 2:1:1 (Cas9 mRNA:modified sgRNA:ssDNA HDR template, 1000 ng total nucleic acid). Each of the respective particle mixtures were added to the cells simultaneously and the same protocol listed above was followed for media addition and incubation time (48 hours). Following incubation, cells were prepared for analysis via flow cytometry as described above.

In vitro HDR titer. HEK293 B/GFP cells were plated in a 12 well tissue culture dish (CytoOne) at a density of 1.5×10⁵ cells per well in 500 μL of DMEM containing 10% FBS and 1% penicillin/streptomycin and allowed to attach overnight at 37° C. After overnight incubation, 4A3-SC8 dLNPs were generated using the ethanol dilution method and contained a mixture of three nucleic acids (Cas9 mRNA, modified sgRNA, ssDNA HDR template) and an internal molar ratio of components fixed at 38.5:30:30:1.5 of 4A3-SC8:cholesterol:DOPE:DMG-PEG. The molar ratio of 4A3-SC8 to nucleic acid was fixed at 2500:1. In all formulations, Cas9 mRNA and modified sgRNA were fixed at one of the following ratios by weight: 1:1, 1:2, or 2:1, wherein 1 signifies 250 ng of that particular nucleic acid. Once the ratio between those two components was fixed, the amount of ssDNA HDR template was added into each individual RNA mixture at one of the following ratios: 0.5, 1, 2, 3, 4, 6, 8, 10. The dLNPs were then administered to the cells using a micropipette. 24 hours after transfection, 1 mL of DMEM was added to each well. Following the 48-hour incubation, cells were prepared for analysis via flow cytometry as described above (N=3+/−standard deviation).

In vitro genomic DNA sequencing. For all cells, gDNA was extracted 48 h post transfection with HDR dLNPs as follows. Briefly, cells were trypsinized at 37° C. for 3 min wherein 500 mL of DMEM was then added to neutralize the trypsin. Cells were then collected in 1.5 mL DNA LoBinid tubes (Eppendorf) and centrifuged at 300 G for 5 min to form a cell pellet. This pellet was then resuspended in cell digestion media. The digestion media consists of 2 μL proteinase K (Ambion, 20 mg/mL), 10 μL of passive cell lysis buffer (Promega), and 40 μL of DEPC treated H₂O (Ambion) per sample. 50 μL of this cell lysis buffer was then added to each cell pellet and the pellet was resuspended using a micropipette. The mixtures were then transferred to 0.5 mL PCR tubes (Fisherbrand) and run on the following PCR cycle: 1× (65° C.—20 min; 95° C.—10 min; 4° C.—hold). The extracted gDNA was then amplified via PCR using the following protocol: 40 μM working solutions of primers B/GFP Fwd 1 and B/GFP Rev 2 were prepared and combined at equal volumes to create a 20 μM mix of both primers. The PCR mixtures for each reaction consisted of 2 μL extracted gDNA from lysed cells, 25 μL DreamTaq Green PCR Master Mix (2×), 1 μL of 20 μM B/GFP Fwd+B/GFP Rev primer mix, and 22 μL DEPC treated H₂O. Each mixture was run at the following cycle conditions: (1× (95° C.—5 min) 35× (95° C.—30 sec; 61° C.—30 sec; 72° C.—1 min) 1× (72° C.—7 min) 1× (4° C.—hold). Following this, PCR purification was performed according to the manufacturer protocol (Qiagen). All samples were then run in a 2% agarose gel at 130 V for 30 min-1 h, verified for correct size, and extracted using Qiagen Gel Purification kit according to the manufacturer's protocol. Purified samples were then submitted for sequencing via Sanger DNA sequencing utilizing a mixture of 6 μL of PCR-amplified DNA at 8.333 ng/μL and 6 μL of 1 μM stock of primer B/GFP Rev 2.

Xenograft HEK293 B/GFP tumor formation. A suspension HEK293 B/GFP cells were resuspended in 1×PBS at a concentration of 1×10⁶ cells per 50 μL and combined with Corning Matrigel Membrane Matrix at a 1:1 volumetric ratio. 100 μL of the mixture was then injected subcutaneously into the right hind leg of athymic nude Foxn1^{nu} mice using a 29G1/2 insulin syringe (Excelint). Tumors were allowed to grow in size for ˜2-3 weeks until measuring 125 mm³.

In vivo dLNP delivery. 4A3-SC8 dLNPs were created for in vivo experiments using the ethanol dilution method and dialyzed against 1×PBS for 2 hours. All dLNPs used for in vivo work were dialyzed against 1×PBS for 2 hours before injection. Molar ratio of internal components was fixed at 38.5:30:30:1.5 of 4A3-SC8:cholesterol:DOPE:DMG-PEG and the mole ratio of 4A3-SC8 to nucleic acid was fixed at 2500:1. The nucleic acid mixture consisted of 1:1:8, 1:1:3, or 2:1:3 fixed ratio by weight of internal components of Cas9 mRNA:modified sgRNA:ssDNA HDR template. 4A3-SC8 dLNPs were injected intratumorally at a dose of 0.5 mg/kg.

Xenograft tumor and organ resection for IVIS imaging. 5 days after injection with HDR dLNPs, the mice were euthanized and the tumor was resected by peeling back the skin. Additionally, the heart, lungs, spleen, and kidneys were resected as well for imaging with IVIS Lumina.

Xenograft tumor preparation for confocal imaging. Resected tumors were placed in a well containing O.C.T. Compound (Tissue-Tek) and then transferred to −80° C. until ready to section (minimum overnight). Tumors frozen in O.C.T. Compound were then placed in the Cryostat (Leica Biosystems) and sectioned at a thickness varying from 7 μm to 15 μm where they were then mounted to a slide. After mounting, the tissue slices were fixed via incubation at RT for 2 hours with 4% paraformaldehyde solution, washed 3× with 1×PBS, and covered.

10× digestion media. Digestion media for tumors consisted of the following mixture: Collagenase I (450 units/μL, Sigma Aldrich), DNAse I (250 units/μL, Sigma Aldrich) and Hyaluronidase (300 units/μL, Sigma Aldrich) in 1×PBS. The mixture was stored at −20° C.

In vivo tumor genomic DNA sequencing. For all tumors, gDNA was extracted from tumors using PureLink Genomic DNA Mini Kit according to manufacturer protocol. The GFP sequence in tumor gDNA was then amplified via PCR. Briefly, 40 μM working solutions of the F′1 and R′2 sequencing primers were prepared and added to a mix consisting of 40 ng gDNA, 25 μL DreamTaq Green Master Mix (2×), 1 μL of F′1 and R′2 primers at a 20 μM concentration, and 22 μL of milliQ H₂O. This mix was run at the following PCR cycle: 1× (95° C.—5 min) 30× (95° C.—30 sec; 62.5° C.—30 sec; 72° C.—1 min) 1× (72° C.—7 min) 1× (4° C.—hold). Samples were then run in a 2% agarose gel at 130 V for 30 min-1 h, verified for correct size, and extracted using Qiagen Gel Purification kit according to the manufacturer's protocol. Purified samples were then submitted for sequencing via Sanger DNA sequencing utilizing a mixture of 6 μL of PCR-amplified DNA at 8.333 ng/μL and 6 μL of 1 μM stock of primer B/GFP Rev 2.

Reference sequence template generation. A reference sequence was generated using the following protocol: gDNA was extracted from PBS treated tumors and amplified using the above PCR cycle. Following amplification and purification, each end of the sequence was amplified using a combination of the F′1 primer+Rev. reference primer, or Fwd. reference primer+R′2 primer for the first half, and second half of the template, respectively. Each of the two reaction mixtures consisted of 1 μL of 20 μM primer mixture, 25 μL of Phusion High-Fidelity PCR Master Mix (2×), 1 ng of PBS PCR product, and H₂O up to 50 μL total reaction volume. The PCR cycle used to generate each half of the template sequence was as follows: 1× (95° C.—5 min) 30× (95° C.—30 sec; 62° C.—30 sec; 72° C.—1 min) 1× (72° C.—7 min) 1× (4° C.—hold). After this amplification, the two sequences were then annealed using the following PCR mixture and cycle: 5 μL of each end of the two reference templates, 25 μL of DreamTaq Green PCR Master Mix (2×), and H₂O up to 50 μL. The PCR cycle was: 1× (95° C.—5 min) 10× (95° C.—30 sec; 62° C.—30 sec; 72° C.—1 min) 1× (72° C.—7 min) 1× (4° C.—hold). Finally, with both ends amplified and annealed together, the resulting PCR product was diluted 200× and then amplified using 1 μL of the 200× diluted PCR product, 1 μL of 20 μM F′1+R′2 primer set, 25 μL of DreamTaq Green PCR Master Mix (2×), and H₂O up to 50 μL under the following conditions: 1× (95° C.—5 min) 30× (95° C.—30 sec; 66° C.—30 sec; 72° C.—1 min) 1× (72° C.—7 min) 1× (4° C.—hold). The resulting product was run in a 2% agarose gel at 130 V for 1 h, purified using the Qiagen Gel Extraction kit, and then sequenced using Sanger DNA Sequencing.

TIDER analysis for HDR via DNA sequencing. The TIDER webtool (tide.nki.nl/#about-tider) was used to calculate HDR from Sanger DNA sequencing data. From TIDER website: “TIDER is a modified version of TIDE that estimates the frequency of targeted small nucleotide changes introduced by CRISPR in combination with homology-directed repair using a donor template. In addition, it determines the spectrum and frequency of non-templated indels. Compared to TIDE, TIDER requires one additional sequencing trace (i.e., three instead of two). Preparation of this third “reference” DNA can be done with a simple two-step PCR protocol. The web tool reports the estimated frequencies of the templated mutation and of all non-templated indels.”

Flow cytometry. For detection of unedited cells (BFP+), NHEJ (no fluorescence), and HDR (GFP+), cells were analyzed with BD LSRFortessa machine (BD Biosciences). GFP+, BFP+, and non-fluorescent cells were quantified using FlowJo.

Statistical analysis. Statistical analysis was performed using a Student's t-test or one-way ANOVA with Tukey's multiple comparisons test or Dunnett's multiple comparisons test in GraphPad Prism.

In vivo Luciferase assay. HEK293 B/GFP cells were used to form tumors in the right hind leg of athymic nude Foxn1^{nu} mice. Once the tumors reached ˜125 mm³ in size, the tumors were injected with 4A3-SC8 dLNPs containing luciferase mRNA at a dose of 0.25 mg/kg. 6 h post intratumoral (IT) injection, the mice were injected intraperitoneally (IP) with d-Luciferin and imaged for luminescence using IVIS. After imaging, the mice were then returned to their housing. 24 h post initial IT injection with dLNPs, the mice were again injected IP with d-Luciferin and imaged using IVIS for luminescence. Immediately following that, whole mouse IVIS imaging, the tumor mass, lung, liver, heart, kidney, and spleen were resected from each mouse for luminescence imaging using IVIS (N=3).

In vivo PTEN off-target assay. HEK293 B/GFP cells were used to form tumors in the right hind leg of athymic nude Foxn1^{nu} mice. Once the tumors reached ˜125 mm³ in size, the tumors were injected with 4A3-SC8 dLNPs containing a 2:1 weight ratio of Cas9 mRNA:PTEN sgRNA at a dose of 0.25 mg/kg. 5 days after IT injection of 4A3-SC8 dLNPs containing Cas9 mRNA and PTEN sgRNA, the liver, lung, and spleen were resected from the mice for downstream off-target analysis (N=3).

In vivo T7E1 and DNA sequencing for PTEN off-target editing. The Cas-OFFinder webtool was used to predict likely off-target editing sites for the PTEN sgRNA. gDNA was extracted from the resected lung, liver, and spleen using the PureLink Genomic DNA mini kit according to the manufacturer protocol. Each of the off-target sites in the gDNA were then amplified via PCR. All samples were then run in a 2% agarose gel at 130 V for 30 min-1 h, verified for correct size, and extracted using Qiagen Gel Purification kit according to the manufacturer's protocol. The gel purified off-target amplicons were then used for the T7E1 assay according to the manufacturer's protocol. Additionally, the gel purified off-target amplicons were submitted for Sanger DNA sequencing (N=3).

In vitro T7E1 and sequencing for off-target HEK293 B/GFP editing. The Cas-OFFinder webtool was used to predict likely off-target editing sites for the B/GFP sgRNA. For all cells, gDNA was extracted 48 h post transfection with HDR dLNPs as follows. Briefly, cells were trypsinized at 37° C. for 3 min wherein 500 mL of DMEM was then added to neutralize the trypsin. Cells were then collected in 1.5 mL DNA LoBinid tubes (Eppendorf) and centrifuged at 300 G for 5 min to form a cell pellet. This pellet was then resuspended in cell digestion media. The digestion media consists of 2 μL proteinase K (Ambion, 20 mg/mL), 10 μL of passive cell lysis buffer (Promega), and 40 μL of DEPC treated H₂O (Ambion) per sample. 50 μL of this cell lysis buffer was then added to each cell pellet and the pellet was resuspended using a micropipette. The mixtures were then transferred to 0.5 mL PCR tubes (Fisherbrand) and run on the following PCR cycle: 1× (65° C.—20 min; 95° C.—10 min; 4° C.—hold). The extracted gDNA was then amplified via PCR at each of the predicted off-target sites and gel purified using the Qiagen Gel Purification Kit according to the manufacturer's protocol. Following amplification, the T7E1 assay was performed using the manufacturer's protocol to check of off-target editing. Additionally, the purified samples were submitted for Sanger DNA sequencing (N=3).

Scramble and commercially available reagent in vitro HDR assessment. HEK293 B/GFP cells were plated in a 12 well tissue culture dish (CytoOne) at a density of 1.5×10⁵ cells per well in 500 μL of DMEM containing 10% FBS and 1% penicillin/streptomycin and allowed to attach overnight at 37° C. After overnight incubation, Lipofectamine 2000 and RNAiMAX lipoplexes were prepared with a 1:1:3 ratio of sgRNA:Cas9 mRNA:ssDNA according to the manufacturer's protocols. Additionally, 4A3-SC8 dLNPs were created containing the following: a 1:1:3 ratio of Cas9 mRNA:sgRNA:ssDNA; a 1:1:3 ratio of Cas9 mRNA:SCsgRNA:ssDNA; a 1:1:3 ratio of Cas9 mRNA:sgRNA:SCssDNA; a 1:3 ratio of Cas9 mRNA:ssDNA (no sgRNA); and a 1:1 ratio of Cas9 mRNA:sgRNA (no ssDNA). All formulations were then administered to the cells. 24 h after transfection, an additional 1 mL of DMEM was added to the cells. Following the 48 h incubation, the cells were prepared for analysis via flow cytometry as previously described (N=3+/−standard deviation).

HDR cytotoxicity analysis. HEK293 B/GFP cells were plated in a 12 well tissue culture dish (CytoOne) at a density of 1.5×10⁵ cells per well in 500 μL of DMEM containing 10% FBS and 1% penicillin/streptomycin and allowed to attach overnight at 37° C. After overnight incubation, Lipofectamine 2000 and RNAiMAX lipoplexes were prepared with a 1:1:3 ratio of sgRNA:Cas9 mRNA:ssDNA according to the manufacturer's protocol. All formulations were then administered to the cells. 24 h after transfection, an additional 1 mL of DMEM was added to the cells. Following the 48 h incubation, the cells were prepared for analysis via flow cytometry as previously described. During preparation for flow cytometry, cells were stained with Ghost Dye Red 780 according to the manufacturer's protocol and washed 3× with PBS thereafter. Cytotoxicity was assessed using flow cytometry (N=3+/−standard deviation; statistical analysis performed using one-way ANOVA with Dunnett's multiple comparisons test against PBS).

### C. Discussion

Progress to date for in vivo editing has largely been limited to gene knockouts via an error-prone DNA repair mechanism known as Non-Homologous End Joining (NHEJ). True correction of genetic disease and cancer mutations will require Homology Directed Repair (HDR), an approach currently hindered by the lack of carriers that can mediate this complex DNA repair pathway. The present disclosure overcomes this challenge by reporting a non-viral, all-in-one approach using dendrimer-based lipid nanoparticles (dLNPs) for precise gene correction by Homology Directed Repair (HDR) in vitro and in vivo.

CRISPR offers several highly desirable traits for gene editing including sequence-dependent target specificity and editing permanence in non-dividing cells (Doudna et al., 2014; Sander and Joung, 2014; Wei et al., 2020). These attributes could, in theory, allow many genetic diseases to be cured by a single treatment. Most in vivo editing efforts so far have utilized NHEJ, whereby insertions and/or deletions (indels) can occur at the site of the Cas9 and Single-Guide RNA (sgRNA) induced Double-Stranded Break (DSB) in the DNA. The introduction of indels into the target site typically results in mutations that subsequently render the protein nonfunctional or truncated (Cong et al., 2013; Ran et al., 2013; Platt et al., 2014; Sanchez-Rivera et al., 2014; Knott and Doudna, 2018). In diseases where knockout of a target is beneficial, the NHEJ mechanism is highly useful. However, in most genetic diseases and cancer applications, HDR correction of the sequence is required for therapeutic benefit (Wei et al., 2020; Lin et al., 2014; Glass et al., 2018; Pickar-Oliver et al., 2019; Yeh et al., 2019; Anzalone et al., 2020; Li et al., 2020; Mitchell et al., 2021). Additionally, in many genetic diseases wherein the mutated genomic sequence codes for a partially active protein that still retains some productive activity, NHEJ could be quite detrimental as it could eliminate previously existing activity.

In contrast, through utilization of HDR, cells can accurately correct the mutated genetic sequence of interest to a precisely fixed sequence. Rather than forming indels at the cut site via NHEJ, the DSB can be repaired when in close proximity to a strand of DNA containing the correct amino acid sequence flanked by 5′ and 3′ regions of overlapping homology to the endogenous DNA (Lin et al., 2014; Pickar-Oliver et al., 2019; Pinder et al., 2015; Song et al., 2016; Gutschner et al., 2016; Jasin and Haber, 2016; Gallagher and Haber, 2018; Aird et al., 2018; Nambiar et al., 2019). In order to utilize this repair process, there are three required components that must work in tandem with one another: Cas9 protein, sgRNA, and a single-stranded DNA (ssDNA) template. Thus, the requirements for HDR correction are much greater than that for NHEJ gene knockouts, which has limited advancement of gene correction efforts.

Due to the difficult challenge of delivering these multiple cargoes, discovery and engineering of delivery systems is an important goal without an obvious solution (Wei et al., 2020; Lin et al., 2014; Glass et al., 2018; Pickar-Oliver et al., 2019; Yeh et al., 2019; Anzalone et al., 2020; Li et al., 2020; Mitchell et al., 2021). While viral vectors are effective for gene delivery, they present several shortcomings for inducing HDR including restrictive packaging limits, risk of random deleterious integration into the genome, and potential immune response (Chandler et al., 2015; Chandler et al., 2017). Among non-viral options for deploying Cas enzymes, delivery of Cas9 mRNA may yield more protein than direct delivery of Cas9 protein on a mass basis and may be safer than delivery of pDNA encoding for Cas9 that could possibly integrate into the host genome (Wang et al., 2017; Wei et al., 2020; Li et al., 2018; Xu et al., 2019; Lattanzi et al., 2019; Mout et al., 2017; Liu et al., 2017). It is believed that a non-viral delivery system for achieving HDR in vivo using a fully nucleic acid-mediated approach has not been reported. Herein, an all-in-one non-viral dendrimer-based lipid nanoparticle (dLNP) system (Zhou et al., 2016; Zhang et al., 2018a; Zhang et al., 2018b; Cheng et al., 2018; Zhou et al., 2020; Cheng et al., 2020; Wei et al., 2020) capable of inducing HDR in vivo is reported

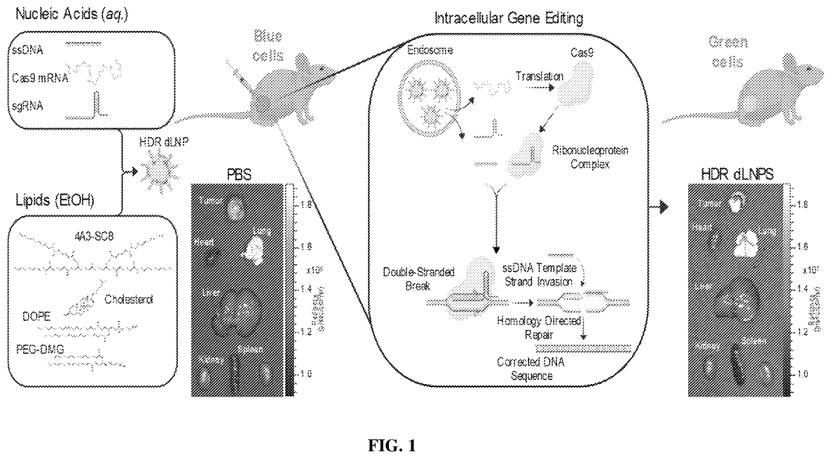

In order to achieve correction of a mutated genetic sequence via HDR, there are challenging barriers that must be overcome (FIG. 1). Herein is presented an approach to an all nucleic acid CRISPR/Cas system consisting of Cas9 mRNA, sgRNA, and donor ssDNA encapsulated in dLNPs to enable cytoplasmic delivery (Zelphati and Szoka, Jr., 1996; Harvie et al., 1998; Hafez et al., 2001; Sahay et al., 2013; Gilleron et al., 2013; Wittrup et al., 2015; Li and Szoka, 2007). After Cas9 mRNA translation, the ribonucleoprotein complex (RNP) (Jinek et al., 2012; Wei et al., 2019; Deltcheva et al., 2011; Gasiunas et al., 2012) is formed, consisting of the Cas9 nuclease and sgRNA, which has been shown to further bind ssDNA (Nguyen et al., 2020) prior to nuclear localization driven by the nuclear localization signal (NLS). RNPs locate the target sequence in the genomic DNA wherein the sgRNA will bind in an anti-parallel complementary fashion upstream of the Protospacer Adjacent Motif (PAM) sequence (Wei et al., 2019; Mojica et al., 2009). Once bound, the Cas9 nuclease will cleave the genomic DNA, resulting in a double-stranded break (Wei et al., 2019; Garneau et al., 2010), wherein the ssDNA HDR donor template can be copied and incorporated into the genomic DNA (FIG. 1) (Lin et al., 2014; Wei et al., 2019; Pinder et al., 2015; Song et al., 2016; Gutschner et al., 2016; Aird et al., 2018; Nambiar et al., 2019). Due to these barriers, an engineering optimization approach was used to identify formulations that can overcome these challenges with a HDR efficiency as the paramount goal. A library of degradable, ionizable dendrimer-based lipids was employed (Zhou et al., 2016) with the ability to be positively charged at low pH to bind RNAs during self-assembly, uncharged at neutral pH to reduce toxicity, and positively charged again at the maturing endosome pH to facilitate endosomal release. Prior work exploring ionizable dendrimer lipids to deliver short siRNAs/miRNAs (18-22 bp) (Zhou et al., 2016; Zhang et al., 2018a; Zhang et al., 2018b; Zhou et al., 2020) or long messenger RNAs (>1000 nt) (Cheng et al., 2018; Cheng et al., 2020) have revealed that creation of new lipid designs (Miller et al., 2017; Miller et al., 2018) and formulation reengineering (Cheng et al., 2018; Cheng et al., 2020) are necessary approaches to develop carriers for new opportunities, but that the solutions to solving the challenges are not obvious. Indeed, it has been shown that optimal carriers for short RNAs are not always effective for long RNAs (Wei et al., 2020; Miller et al., 2017; Li et al., 2015; Kauffman et al., 2015; Miller and Siegwart, 2018; Ball et al., 2018; Hajj et al., 2019; Patel et al., 2017; Hao et al., 2015). Such efforts on LNP engineering have not yet been directed towards HDR-mediated genome correction.

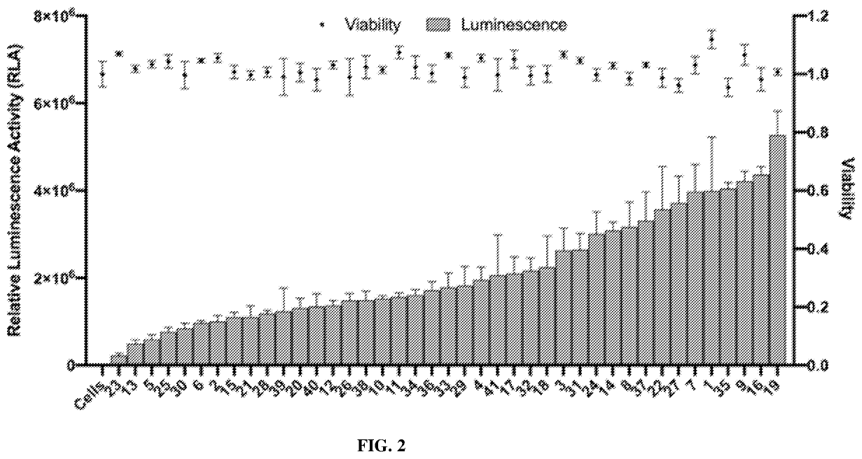

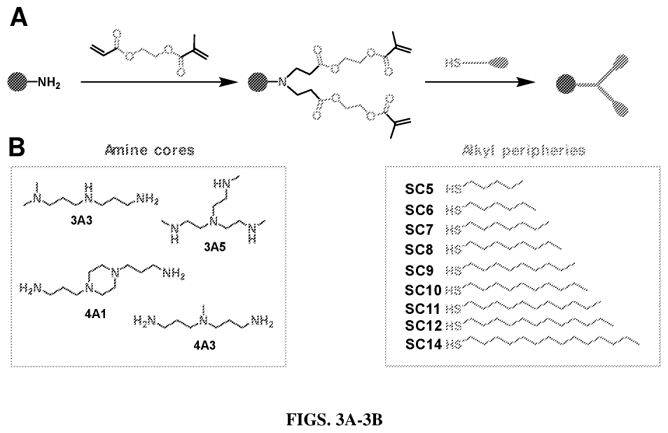

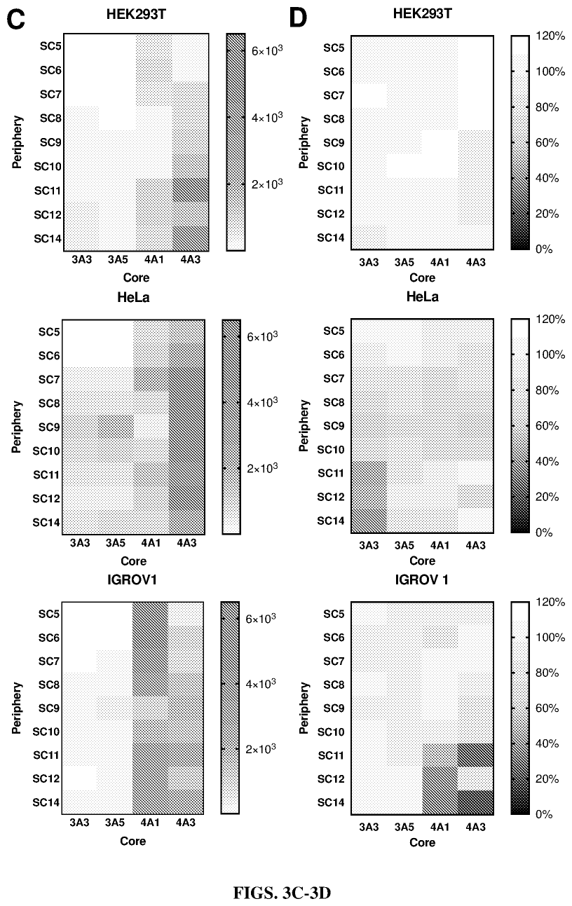

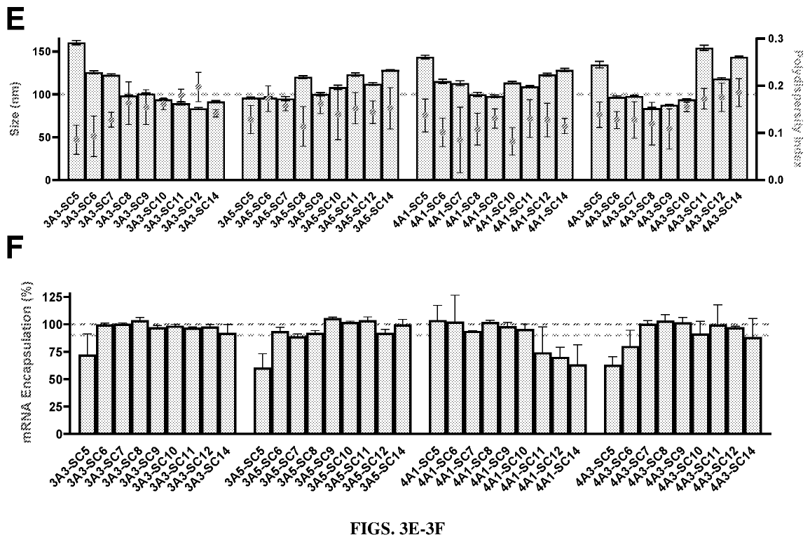

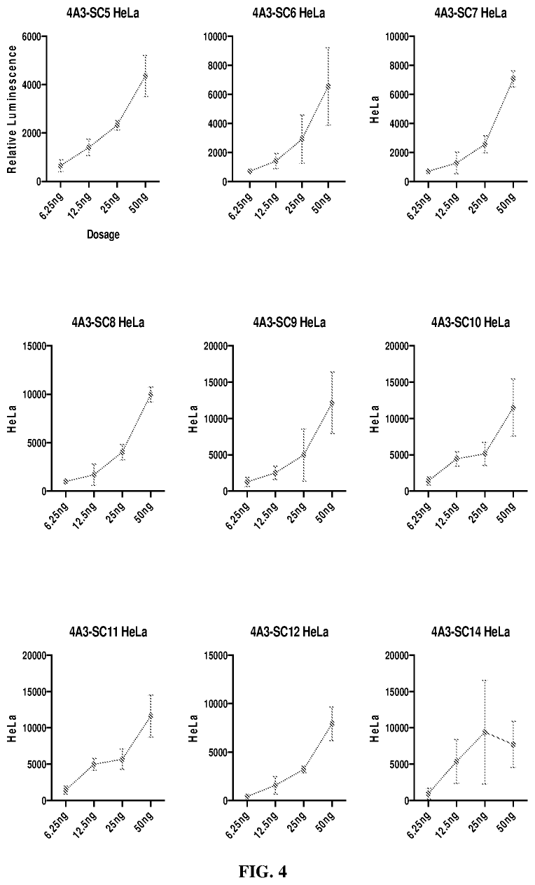

Here, specific focus was directed toward co-encapsulation of three components (Cas9 mRNA, sgRNA, and donor DNA), which presented an unprecedented mixture of nucleic acids of different length and chemical composition. It was reasoned that the overall physical properties of the combined nucleic acid cargoes would exhibit hydrophobic and electrostatic characteristics more similar to long RNAs than short RNAs (Zhou et al., 2016). Focus was directed to altering the molar ratios of the four core lipid components (ionizable amino dendrimer lipid, amphipathic phospholipid, cholesterol, PEG2000-DMG) within dLNPs, which can drastically alter their ability to effectively deliver short (siRNA) to long (mRNA) cargoes (Cheng et al., 2018). Initial focus was directed toward dendrimers and formulation parameters (38.5:30:30:1.5; Dendrimer:Cholestserol:DOPE:PEG-DMG) with the most flexibility across nucleic acid types (FIG. 2). The screening library of dendrimers that were selected for analysis consisted of four distinct amine cores (3A3, 3A5, 4A1, 4A3) and nine peripheries with different alkyl chain lengths (SC5, SC6, SC7, SC8, SC9, SC10, SC11, SC12, SC14) totaling 36 individual dendrimer lipids (FIGS. 3A & 3B) (Zhou et al., 2016). Each of the dendrimers were then combined with cholesterol, DOPE, and PEG-DMG in ethanol at the optimized internal molar ratios and rapidly mixed with luciferase (Luc) mRNA in an acidic aqueous buffer to form dLNPs (Table 2).

TABLE 2
The internal molar ratios and particle percentage breakdown of the individual components
as well as the fixed molar ratios of dendrimer lipid component to nucleic acid.

| | Dendrimer: | Molar Ratio | | | | Lipid Percentage | | | |
|---|---|---|---|---|---|---|---|---|---|
| | NA MR | 4A3-5C | Cholesterol | DOPE | DMG-PEG | 4A3-5C | Cholesterol | DOPE | DMG-PEG |
| 1 | 2000 | 30 | 40 | 40 | 1.5 | 26.91% | 35.87% | 35.87% | 1.35% |
| 2 | 2000 | 70 | 40 | 40 | 1.5 | 46.20% | 26.40% | 26.40% | 0.99% |
| 3 | 5000 | 30 | 40 | 40 | 1.5 | 26.91% | 35.87% | 35.87% | 1.35% |
| 4 | 5000 | 70 | 40 | 40 | 1.5 | 46.20% | 26.40% | 26.40% | 0.99% |
| 5 | 4000 | 50 | 20 | 20 | 1.5 | 54.64% | 21.86% | 21.86% | 1.64% |
| 6 | 4000 | 50 | 20 | 60 | 1.5 | 38.02% | 15.21% | 45.63% | 1.14% |
| 7 | 4000 | 50 | 60 | 20 | 1.5 | 38.02% | 45.63% | 15.21% | 1.14% |
| 8 | 4000 | 50 | 60 | 60 | 1.5 | 29.15% | 34.99% | 34.99% | 0.87% |
| 9 | 4000 | 30 | 40 | 40 | 0.5 | 27.15% | 36.20% | 36.20% | 0.45% |
| 10 | 4000 | 30 | 40 | 40 | 2.5 | 26.67% | 35.56% | 35.56% | 2.22% |
| 11 | 4000 | 70 | 40 | 40 | 0.5 | 46.51% | 26.58% | 26.58% | 0.33% |
| 12 | 4000 | 70 | 40 | 40 | 2.5 | 45.90% | 26.23% | 26.23% | 1.64% |
| 13 | 2000 | 50 | 20 | 40 | 1.5 | 44.84% | 17.94% | 35.87% | 1.35% |
| 14 | 2000 | 50 | 60 | 40 | 1.5 | 33.00% | 39.60% | 26.40% | 0.99% |
| 15 | 6000 | 50 | 20 | 40 | 1.5 | 44.84% | 17.94% | 35.87% | 1.35% |
| 16 | 6000 | 50 | 60 | 40 | 1.5 | 33.00% | 39.60% | 26.40% | 0.99% |
| 17 | 4000 | 50 | 40 | 20 | 0.5 | 45.25% | 36.20% | 18.10% | 0.45% |
| 18 | 4000 | 50 | 40 | 20 | 2.5 | 44.44% | 35.56% | 17.78% | 2.22% |
| 19 | 4000 | 50 | 40 | 60 | 0.5 | 33.22% | 26.58% | 39.87% | 0.33% |
| 20 | 4000 | 50 | 40 | 60 | 2.5 | 32.79% | 26.23% | 39.34% | 1.64% |
| 21 | 4000 | 30 | 20 | 40 | 1.5 | 32.79% | 21.86% | 43.72% | 1.64% |
| 22 | 4000 | 30 | 60 | 40 | 1.5 | 22.81% | 45.63% | 30.42% | 1.14% |
| 23 | 4000 | 70 | 20 | 40 | 1.5 | 53.23% | 15.21% | 30.42% | 1.14% |
| 24 | 4000 | 70 | 60 | 40 | 1.5 | 40.82% | 34.99% | 23.32% | 0.87% |
| 25 | 2000 | 50 | 40 | 20 | 1.5 | 44.84% | 35.87% | 17.94% | 1.35% |
| 26 | 2000 | 50 | 40 | 60 | 1.5 | 33.00% | 26.40% | 39.60% | 0.99% |
| 27 | 6000 | 50 | 40 | 20 | 1.5 | 44.84% | 35.87% | 17.94% | 1.35% |
| 28 | 6000 | 50 | 40 | 60 | 1.5 | 33.00% | 26.40% | 39.00% | 0.99% |
| 29 | 4000 | 50 | 20 | 40 | 0.5 | 45.25% | 18.10% | 36.20% | 0.45% |
| 30 | 4000 | 50 | 20 | 40 | 2.5 | 44.44% | 17.78% | 35.56% | 2.22% |
| 31 | 4000 | 50 | 60 | 40 | 0.5 | 33.22% | 39.87% | 26.58% | 0.33% |
| 32 | 4000 | 50 | 60 | 40 | 2.5 | 32.79% | 39.34% | 26.23% | 1.64% |
| 33 | 2000 | 50 | 40 | 40 | 0.5 | 38.31% | 30.65% | 30.65% | 0.38% |
| 34 | 2000 | 50 | 40 | 40 | 2.5 | 37.74% | 30.19% | 30.19% | 1.89% |
| 35 | 6000 | 50 | 40 | 40 | 0.5 | 38.31% | 30.65% | 30.65% | 0.38% |
| 36 | 6000 | 50 | 40 | 40 | 2.5 | 37.74% | 30.19% | 30.19% | 1.89% |
| 37 | 4000 | 30 | 40 | 20 | 1.5 | 32.79% | 43.72% | 21.86% | 1.64% |
| 38 | 4000 | 30 | 40 | 60 | 1.5 | 22.81% | 30.42% | 45.63% | 1.14% |
| 39 | 4000 | 70 | 40 | 20 | 1.5 | 53.23% | 30.42% | 15.21% | 1.14% |
| 40 | 4000 | 70 | 40 | 60 | 1.5 | 40.82% | 23.32% | 34.99% | 0.87% |
| 41 | 4000 | 50 | 40 | 40 | 1.5 | 38.02% | 30.42% | 30.42% | 1.14% |

Since non-viral nanoparticles can exhibit some degree of cell-type specificity (Yan et al., 2016), the library of selected dendrimer compounds was screened for both effective delivery—as quantified by luciferase expression—as well as low toxicity across three different cell lines (HEK293T, HeLa, and JGROV-1) to identify the most active formulation across multiple cell types (FIGS. 3C & 3D)). Additionally, to rule out any discrepancies in delivery that may be due to ineffective nucleic acid binding or distortions during particle formation, RNA encapsulation efficiency and particle size and uniformity were assessed using the Ribogreen assay and dynamic light scattering (DLS), respectively. As expected, there were no major differences observed with respect to dLNP size, as most averaged roughly 100 nm in diameter; all nanoparticles were uniform (PDI<0.2) (FIG. 2E); and most dLNPs effectively encapsulated >92% of mRNA at the tested ratios (FIG. 2F). In contrast to the lack of variance observed with respect to these aspects of the different dLNPs, notable and surprising differences in delivery efficacy were illuminated between dendrimer compounds. With the goal set to maximize HDR across cell lines, the highest delivery for formulations containing dendrimers with 4A1 and 4A3 amine cores were noted. Increased alkyl chain length dependence was observed in IGROV1 and HEK293T cells. In consideration of the data as a whole, 4A3 emerged as a lead amine core for long nucleic acid delivery due to its activity across all three cell lines, including marked efficacy in HeLa cells.

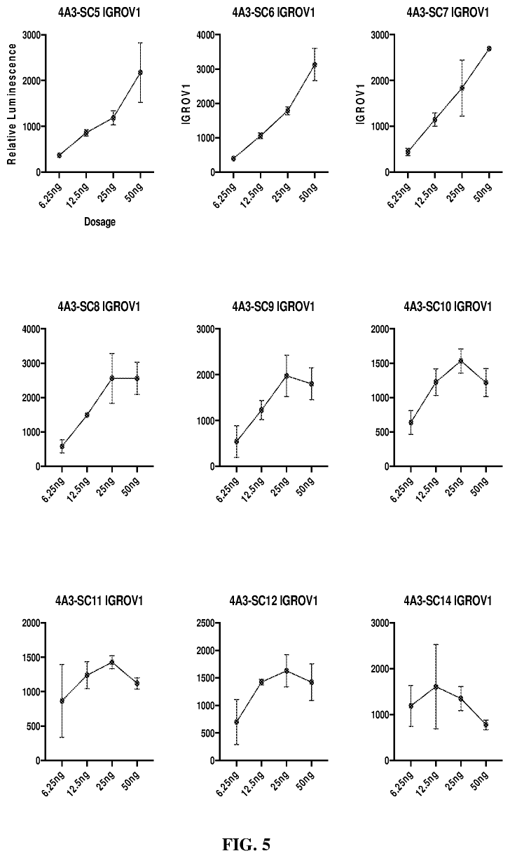

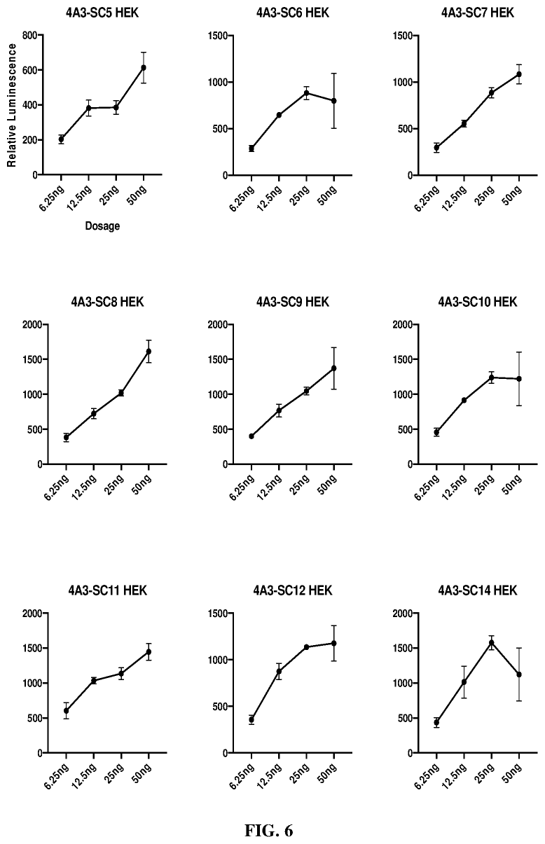

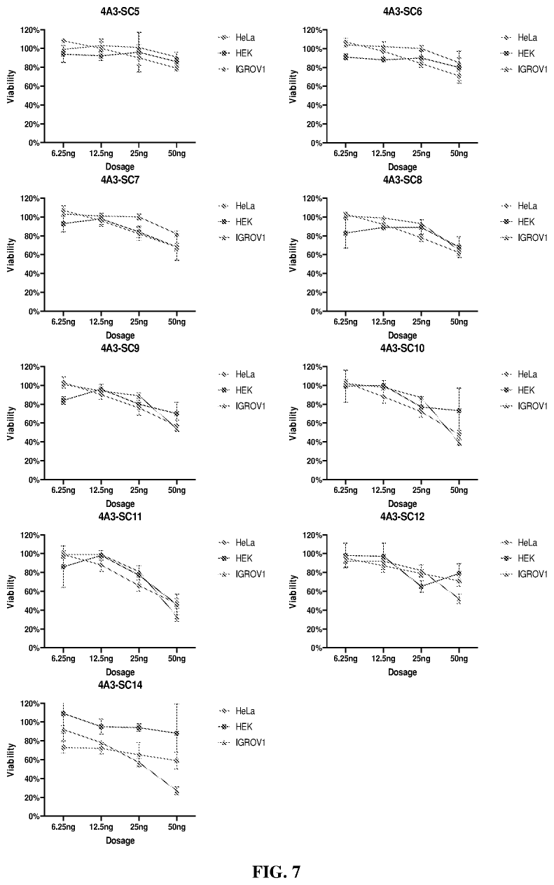

To further define the role of alkyl chain length in delivery efficiency and toxicity, a dose-response assay was conducted using the top amine core candidate (4A3) and all previously examined alkyl peripheries (SC5-SC14) across the same three cell lines (FIGS. 4-6). A slight trend emerged between increasing alkyl chain length and delivery efficacy. However, some mild increases in cytotoxicity were also observed with increasing alkyl chain length (FIG. 7). In consideration of these two factors, it was concluded that 4A3-SC8 dLNPs demonstrated an optimal balance of pronounced delivery efficacy and minimal toxicity (FIG. 3).

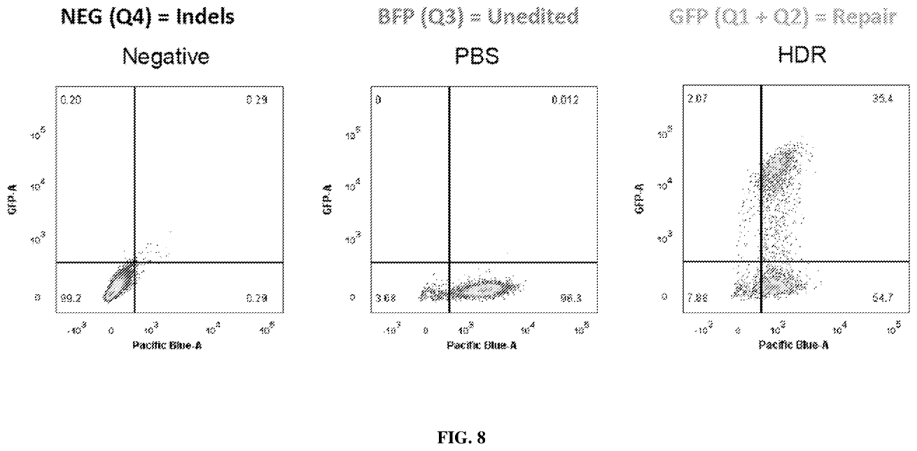

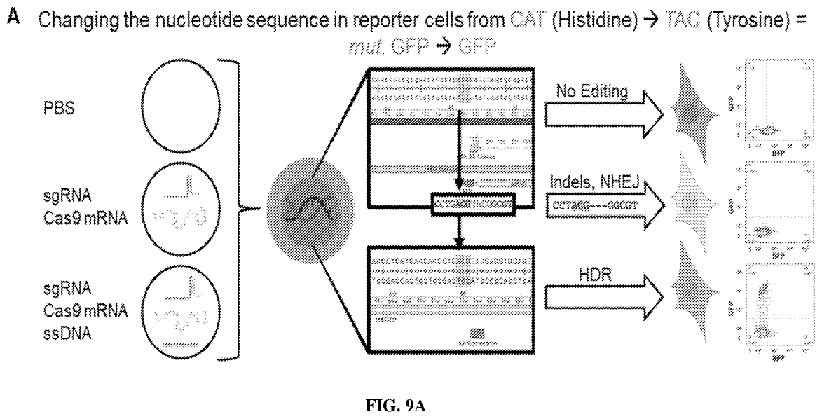

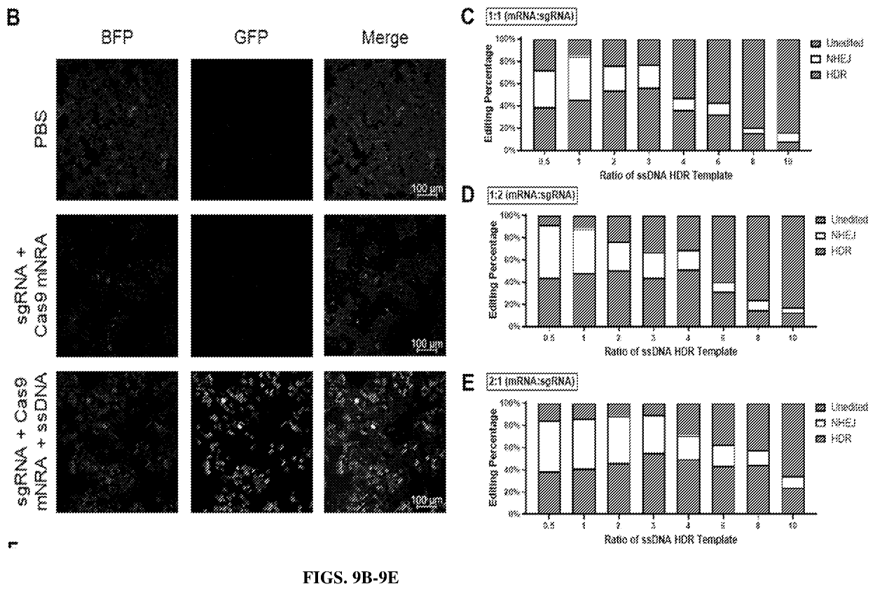

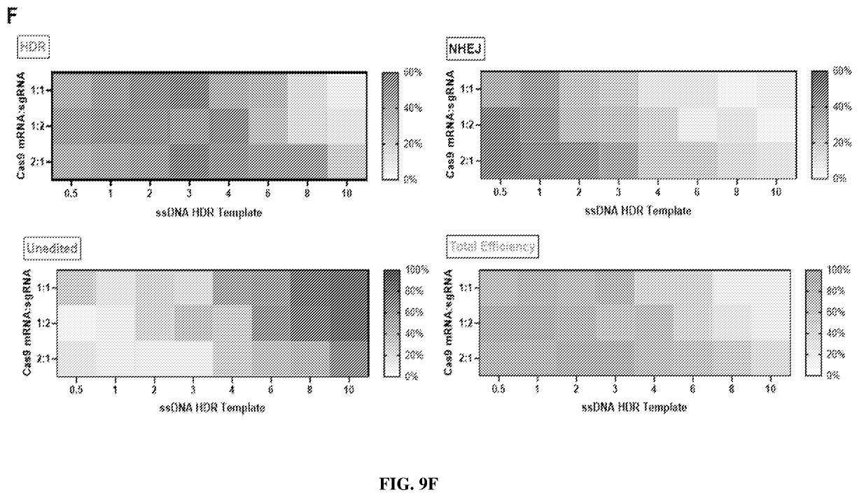

Accomplishing non-viral HDR-mediated gene editing requires synthetic carriers to deliver nucleic acids with very large differences in size: Cas9 mRNA (˜4500 nt), modified sgRNA (˜120 nt), and ssDNA HDR template (127 nt) (FIG. 1). This challenge is further compounded by the fact that Cas9 mRNA must first be translated into Cas9 protein before it can accomplish guided gene editing. To address these factors using the optimized 4A3-SC8 dLNPs, systematic analysis of both the kinetics of cargo delivery with respect to protein expression and the ability of 4A3-SC8 dLNPs to encapsulate multiple nucleic acids inside of a single nanoparticle was performed. HEK293 cells expressing a GFP sequence with a single Y66H amino acid mutation (CAT in place of TAC) were employed to quantify NHEJ and HDR events (Richardson et al., 2016). With this mutated sequence (CAT), the cells fluoresce blue instead of green; however, when the mutation is corrected to TAC, the cells regain normal green fluorescence (FIG. 8). As such, insertion of the correct amino acid sequence into this position restores GFP function. If indels are present in the sequence, (indicative of NHEJ), the cells lose their fluorescence (FIGS. 9A & 9B) (Richardson et al., 2016).

Staged and simultaneous delivery approaches were compared to identify the most convenient and effective approach for HDR correction. Because Cas9 is delivered in the form of mRNA, it was contemplated that staged delivery using two or three separate dLNPs could aid HDR by allowing time for mRNA translation to protein. On the other hand, simultaneous co-delivery of all three nucleic acids in one nanoparticle would be more convenient and translatable to in vivo editing.

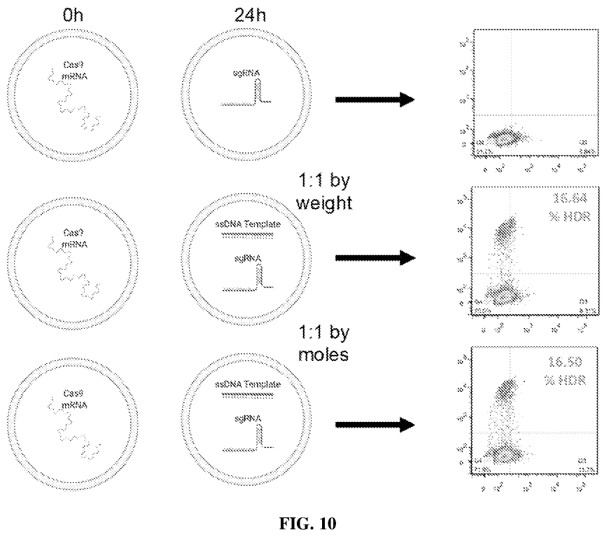

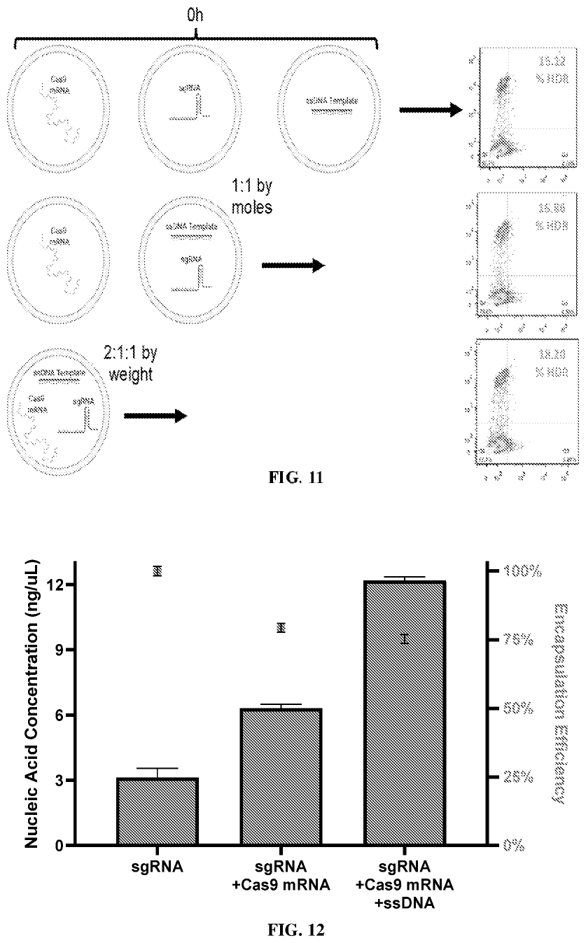

First, to test the staged two-particle approach, 4A3-SC8 dLNPs containing Cas9 mRNA were administered to HEK293 B/GFP cells followed 24 h later by 4A3-SC8 dLNPs containing both modified sgRNA and ssDNA HDR template in a single nanoparticle at fixed ratios of 1:1, either by weight or by moles (FIG. 10). Second, to test the three-particle approach, each set of dLNPs contained only one of the following: Cas9 mRNA, modified sgRNA, or ssDNA HDR template. Third, the approach of an all-in-one simultaneous dLNP delivery was tested wherein a single dLNP formulation contained all three nucleic acid components for HDR (FIG. 11). To confirm that all three nucleic acids (Cas9 mRNA, sgRNA, ssDNA) required for HDR were co-encapsulated into a single dLNP formulation, nucleic acid loading was quantified and encapsulation verified (FIG. 12). Encouragingly, HDR was achieved by all three approaches as evaluated by GFP signal using flow cytometry at a similar efficiency of ˜18% (FIG. 10 and FIG. 11). Although all three approaches were viable, hereafter focus was directed toward all-in-one simultaneous dLNP delivery because it was the most facile and efficacy was not decreased when compared with the staged approaches. Simultaneous, one pot delivery is especially valuable when considering accomplishing non-viral HDR in vivo as it ensures all three components will be internalized into an individual target cell, rather than only one or two of the necessary components.

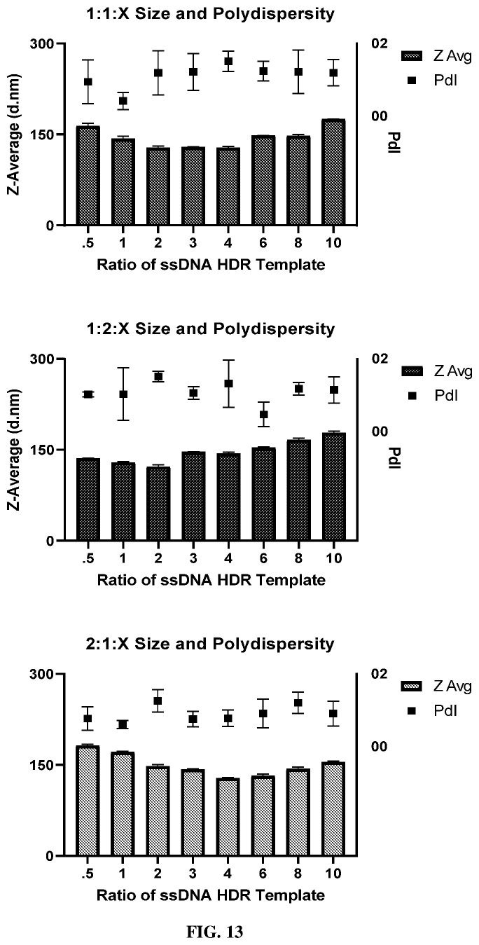

Building on the all-in-one nanoparticle approach, improvement of HDR gene correction was sought by optimizing the ratio of nucleic acids within the dLNPs. Sets of 4A3-SC8 dLNPs were created wherein Cas9 mRNA and sgRNA were fixed at ratios of 1:1 (FIG. 9C), 1:2 (FIG. 9D), and 2:1 (FIG. 9E), respectively, by weight. Then, with the hypothesis that with more ssDNA HDR template available, cells could achieve a higher amount of correction via HDR, the ssDNA HDR template was titrated into the nucleic acid mixtures at increasing ratios of 0.5, 1, 2, 3, 4, 6, 8, and 10 (Table 1). Among all groups tested, the ratios of 1:1:3 and 2:1:3 mRNA:modified sgRNA:ssDNA HDR template were the most efficacious, both resulting in similar HDR correction rates of 56% as quantified via DNA sequencing. DNA sequencing was instituted as the primary analytical technique for detecting gene editing events because it provides an unambiguous quantification of nucleic acid modifications at single base resolution in DNA, thereby avoiding any bias that may be associated with fluorescence reporter techniques. Interestingly, the amount of HDR achieved in all groups (1:1, 1:2, and 2:1) appeared to hit a corrective maxima of ˜50% HDR when the ratio of ssDNA HDR template included in the HDR dLNPs was between 3 and 4. In the groups where Cas9 mRNA and sgRNA were fixed at ratios of 1:1 and 1:2, and the amount of ssDNA in each group was fixed at ratios of 3 and 4, respectively, the amount of HDR correction induced dramatically tapered off. However, in the group where Cas9 mRNA and sgRNA were fixed at a ratio of 2:1, the amount of HDR achieved declined as the ratio of ssDNA HDR template increased beyond 3, but not nearly as substantially nor as sharply as in the 1:1 and 1:2 groups (FIG. 9E). When analyzed for the amount and type of editing achieved across the three Cas9 mRNA:sgRNA fixed groups, common trends emerged with a progressive increase in total editing efficiency, HDR, and NHEJ up until a ssDNA ratio of ˜4 wherein editing efficiency began to decline and the number of unedited cells started to increase. Notably, editing of all forms also appeared to decline more gradually after a ssDNA ratio of 4 in the 1:2 and 2:1 groups (FIG. 9F), which was surprising. In agreement with previous adenovirus (AAV) delivery data, these results indicate that Cas9 may not be the limiting factor for inducing high levels of HDR-mediated correction (Min et al., 2019b). Rather, the availability of the ssDNA template proximal to the cut site may be more important to increasing HDR, wherein an optimal ratio led to the highest HDR balancing the total amount of all three components (Cas9 mRNA, sgRNA, ssDNA). For all ratios, HDR dLNP size and polydispersity were measured to identify whether any correlation existed between HDR efficiency and either of these characteristics. No notable differences existed between formulations with all HDR dLNPs exhibiting uniformity and averaging ˜150 nm in diameter (FIG. 13).

TABLE 1
The ratios of nucleic acids within each formulation of HDR dLNPs.

| | Cas9 mRNA | | SSDNA HDR Temp. | Total Nucleic |
|---|---|---|---|---|
| Ratio | (ng) | sgRNA (ng) | (ng) | Acid (ng) |
| 1:1:0.5 | 250 | 250 | 125 | 625 |
| 1:1:1 | 250 | 250 | 250 | 750 |
| 1:1:2 | 250 | 250 | 500 | 1000 |
| 1:1:3 | 250 | 250 | 750 | 1250 |
| 1:1:4 | 250 | 250 | 1000 | 1500 |
| 1:1:6 | 250 | 250 | 1500 | 2000 |
| 1:1:8 | 250 | 250 | 2000 | 2500 |
| 1:1:10 | 250 | 250 | 2500 | 3000 |
| 1:2:0.5 | 250 | 500 | 125 | 875 |
| 1:2:1 | 250 | 500 | 250 | 1000 |
| 1:2:2 | 250 | 500 | 500 | 1250 |
| 1:2:3 | 250 | 500 | 750 | 1500 |
| 1:2:4 | 250 | 500 | 1000 | 1750 |
| 1:2:6 | 250 | 500 | 1500 | 2250 |
| 1:2:8 | 250 | 500 | 2000 | 2750 |
| 1:2:10 | 250 | 500 | 2500 | 3250 |
| 2:1:0.5 | 500 | 250 | 125 | 875 |
| 2:1:1 | 500 | 250 | 250 | 1000 |
| 2:1:2 | 500 | 250 | 500 | 1250 |
| 2:1:3 | 500 | 250 | 750 | 1500 |
| 2:1:4 | 500 | 250 | 1000 | 1750 |
| 2:1:6 | 500 | 250 | 1500 | 2250 |
| 2:1:8 | 500 | 250 | 2000 | 2750 |
| 2:1:10 | 500 | 250 | 2500 | 3250 |

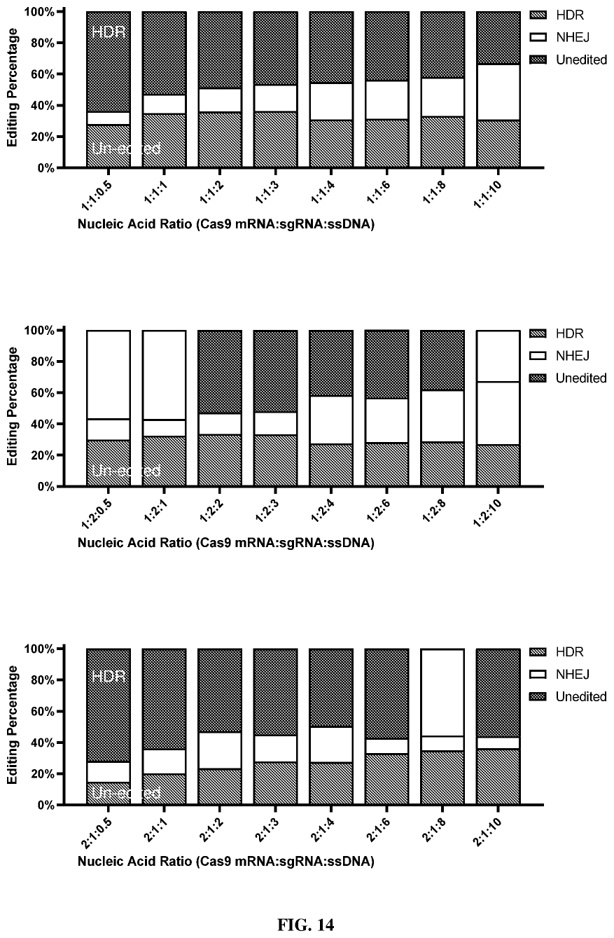

To confirm the results of DNA sequencing, HDR correction was also measured using flow cytometry. The amount of HDR achieved in the groups where Cas9 mRNA and sgRNA were fixed at ratios of 1:1 and 1:2 appeared to be somewhat independent of the amount of ssDNA HDR template included in the dLNPs with conditions inducing HDR at a rate of 30-35%, which could be related to the random viral integration of B/GFP DNA into the model HEK cells, although more work will be needed to study this further. When Cas9 mRNA and modified sgRNA were fixed at ratios of 2:1, there was a nearly linear increase the amount of HDR achieved in concordance with an increase in ssDNA HDR template. Notably, although there was not a large variance in the amount of HDR achieved in the 1:1 and 1:2 Cas9 mRNA:sgRNA groups, many of the formulations appeared to hit a similar asymptote that mirrored the maxima of ˜36% HDR achieved in the 2:1 Cas9 mRNA:sgRNA group with respect to amount of HDR induced (FIG. 14). To further confirm correction of the amino acid sequence and visualize the ability of the 4A3-SC8 dLNPs to induce HDR, nanoparticles were loaded Cas9 mRNA, modified sgRNA, and ssDNA HDR template at a ratio of 2:1:1 and cells were imaged using confocal microscopy (FIG. 9B). As expected, corrected cells were abundant and expressing bright GFP signal which was notably absent in the PBS control.

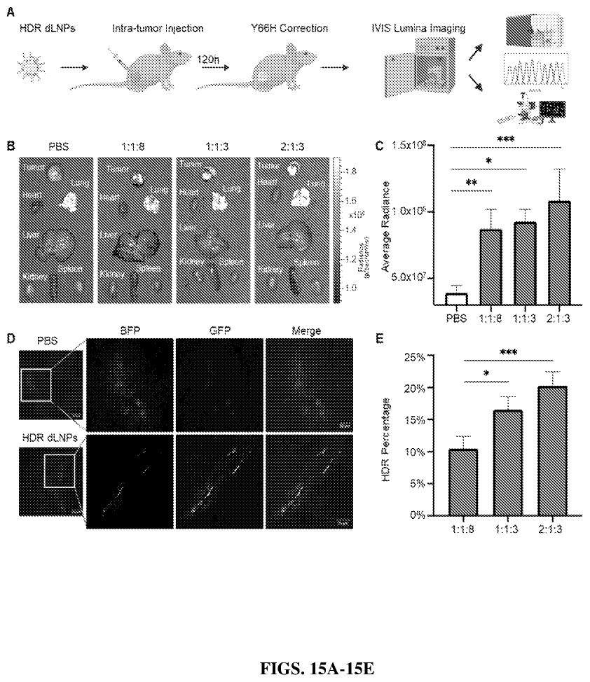

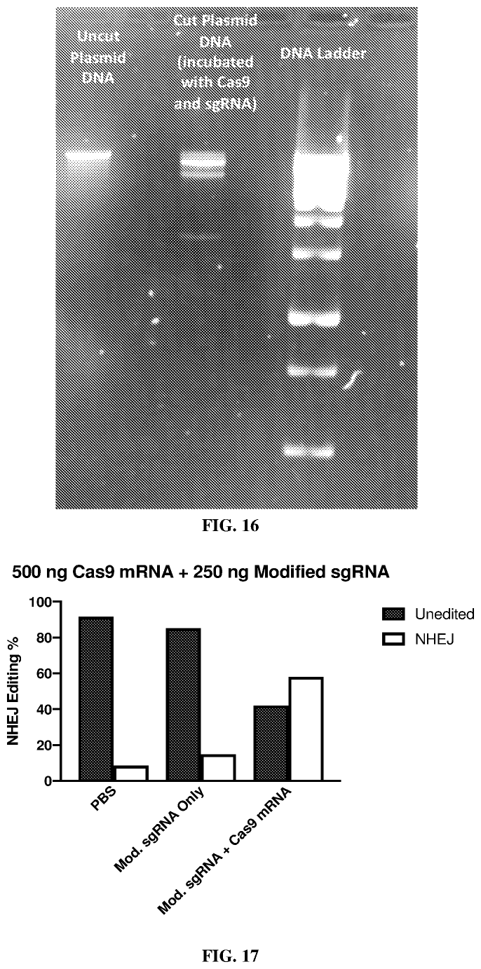

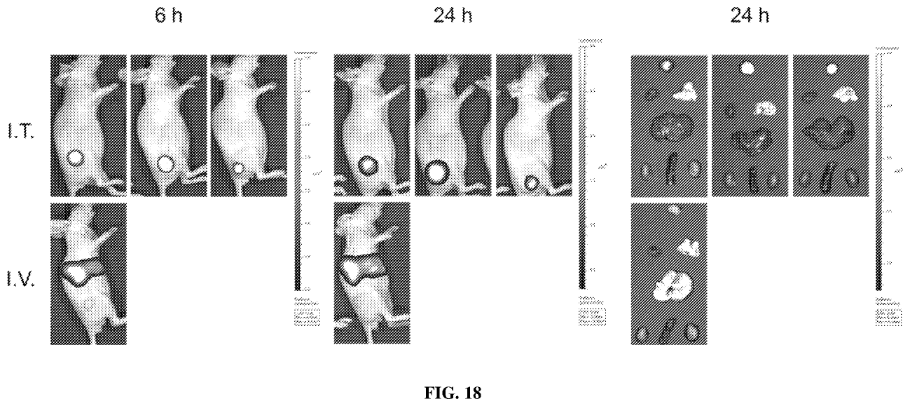

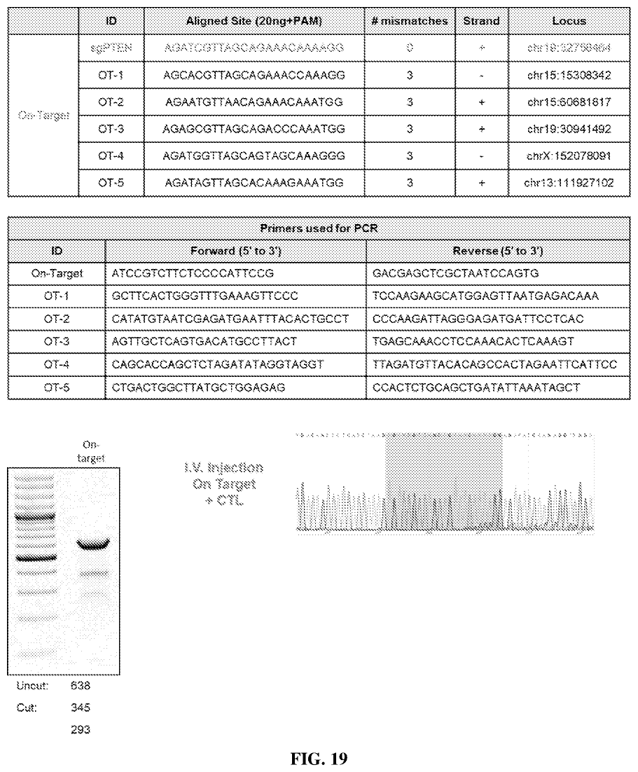

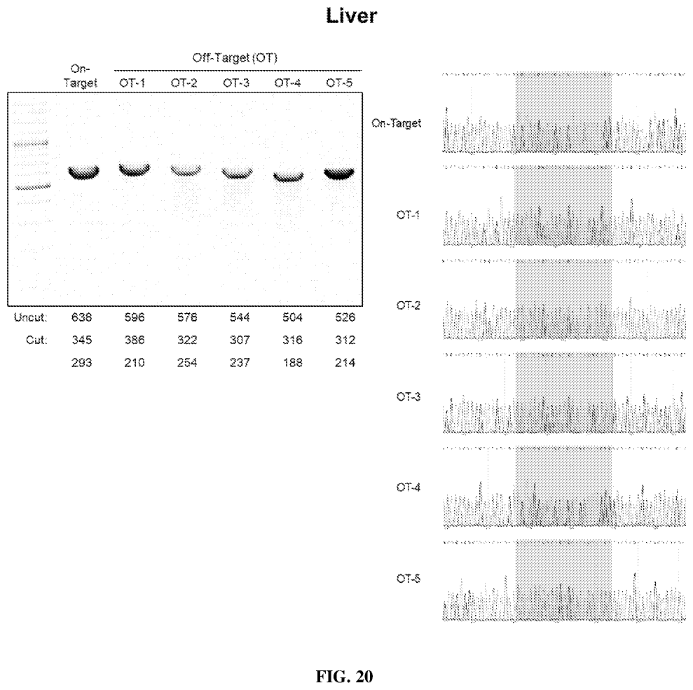

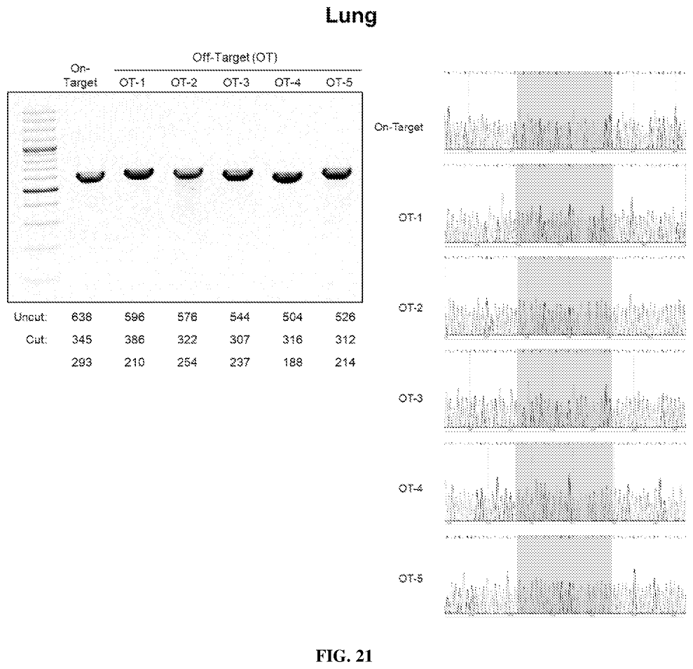

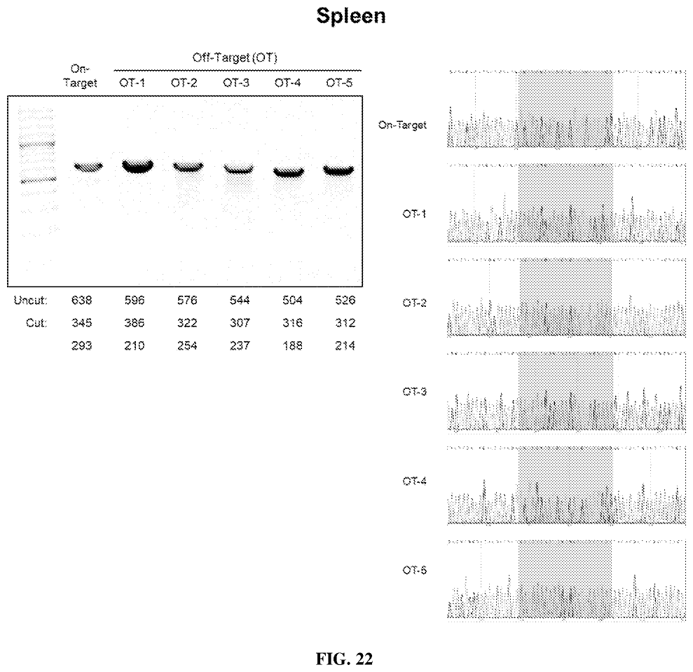

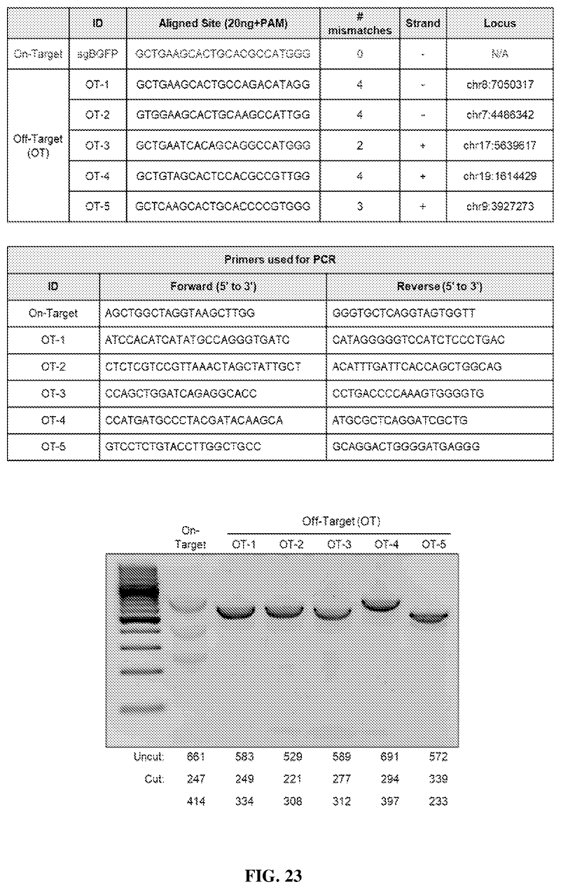

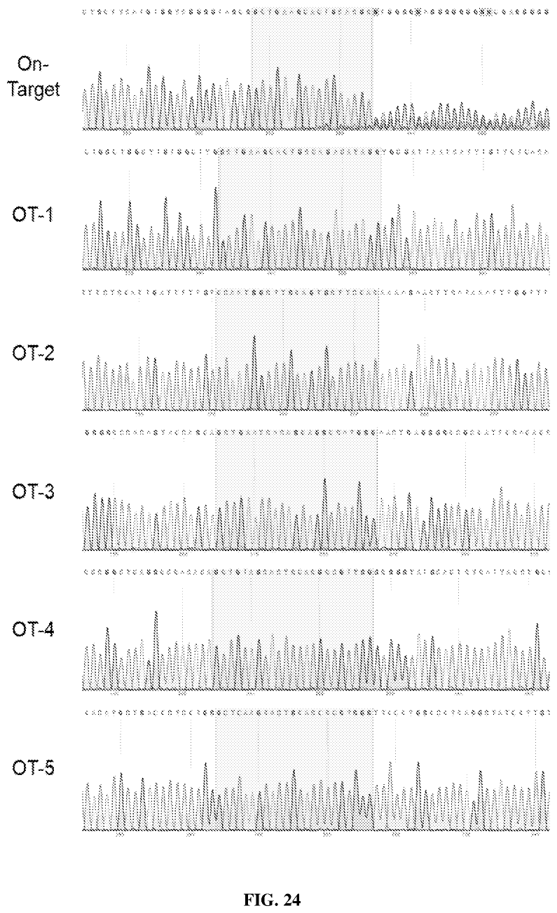

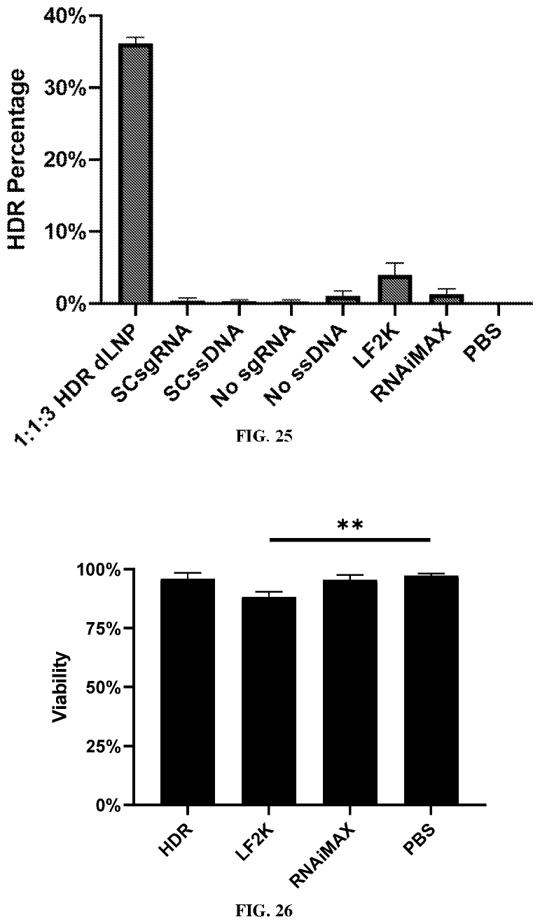

With an established system for accomplishing high levels of HDR in vitro, the ability of 4A3-SC8 dLNPs containing nucleic acid ratios of either 1:1:8, 1:1:3, or 2:1:3 Cas9 mRNA:sgRNA:ssDNA, respectively, were next evaluated to induce HDR in vivo in a proof-of-principle experiment to aid future translatable disease correction. Xenograft tumors were generated utilizing a 1:1 mixture of the HEK293 B/GFP cells and Matrigel, which was then injected subcutaneously in the right hind legs of athymic nude Foxn1^{nu} mice. Once the tumors were 25 mm³ in size, each set of 4A3-SC8 dLNPs containing one of the three ratios (1:1:8, 1:1:3, 2:1:3) of Cas9 mRNA, modified sgRNA, and ssDNA HDR template were formulated and injected intratumorally at a dose of 0.5 mg/kg total nucleic acids. After 5 days, the tumors along with internal organs were resected and imaged using IVIS for GFP signal (FIG. 15A). Bright green GFP signal, indicative of gene correction via HDR, was readily apparent in the tumors treated with 4A3-SC8 dLNPs containing HDR machinery, compared with no detectable GFP signal in the PBS control (FIGS. 15B & 15C). In addition to IVIS imaging, tumors were sectioned and imaged using confocal microscopy. As expected, the tumors again demonstrated bright GFP signal indicating that HDR had been achieved (FIG. 15D).

With visual confirmation that HDR had been accomplished in vivo, tumors were further analyzed via DNA sequencing to accurately quantify HDR correction throughout the tumor at the single nucleotide level. It was reasoned that DNA extraction from the entire tumor tissue would accurately reflect total editing in an unbiased fashion. Moreover, since the HEK293 (Y66H) cells were created via transduction with a lentivirus, the copy number and insertion site of the mutated GFP reporter sequence may vary between cells. Thus, DNA sequencing may eliminate these variables by quantifying all copies of the mutant GFP sequence in cells. DNA sequencing of genomic DNA extracted from tumors revealed HDR editing of up to 23% in the group treated with 2:1:3 HDR dLNPS after analysis using TIDER (Tracking of Insertions, DEletions, and Recombination events) for assessing gene editing (FIG. 15E). It is believed that in vivo HDR corrections rates have to date been limited to 1-5%, which in some cases required combination of viral and non-viral delivery or repeated local injections (Jo et al., 2019; Yin et al., 2014; Yin et al., 2016; Lee et al., 2017). Here it is shown that a single injection of HDR dLNPs into the tumor at a dose of 0.5 mg/kg yielded >20% HDR-mediated gene correction. Moreover, dLNPs are able to overcome the avascularity, large size, and stiffness of the tumors to mediate HDR in vivo.

TABLE 3
The p values associated with in vitro HDR percentage as
determined by TIDER analysis of DNA sanger sequencing are
reported above for each of the replicates (N = 3).

| Cas9 mRNA:sgRNA:ssDNA | N = 1 | N = 2 | N = 3 |
|---|---|---|---|
| 1:1:0.5 | 2.00E−120 | 7.60E−54 | 1.60E−191 |
| 1:1:1 | 1.90E−290 | 1.60E−70 | 6.20E−216 |
| 1:1:2 | 8.00E−220 | 0.00E+00 | 8.70E−295 |
| 1:1:3 | 0.00E+00 | 0.00E+00 | 0.00E+00 |
| 1:1:4 | 7.30E−246 | 1.30E−281 | 6.00E−222 |
| 1:1:6 | 2.20E−102 | 1.40E−41 | 7.30E−130 |
| 1:1:8 | 1.40E−59 | 6.50E−141 | 9.60E−18 |
| 1:1:10 | 3.70E−02 | 3.60E−29 | 0.00E+00 |
| 1:2:0.5 | 2.70E−98 | 6.10E−80 | 5.90E−166 |
| 1:2:1 | 1.40E−119 | 1.40E−137 | 1.40E−118 |
| 1:2:2 | 0.00E+00 | 0.00E+00 | 4.10E−171 |
| 1:2:3 | 1.50E−144 | 3.30E−117 | 1.80E−143 |
| 1:2:4 | 0.00E+00 | 0.00E+00 | 0.00E+00 |
| 1:2:6 | 2.00E−261 | 2.00E−88 | 5.70E−176 |
| 1:2:8 | 4.10E−16 | 1.30E−18 | 7.20E−120 |
| 1:2:10 | 6.00E−37 | 8.60E−91 | 1.50E−08 |
| 2:1:0.5 | 1.00E−122 | 2.70E−173 | 4.00E−32 |
| 2:1:1 | 2.30E−124 | 8.80E−142 | 4.10E−41 |
| 2:1:2 | 2.50E−198 | 2.70E−40 | 4.60E−235 |
| 2:1:3 | 2.00E−209 | 2.90E−246 | 5.80E−203 |
| 2:1:4 | 4.30E−123 | 0.00E+00 | 0.00E+00 |
| 2:1:6 | 0.00E+00 | 2.10E−80 | 3.80E−137 |
| 2:1:8 | 0.00E+00 | 0.00E+00 | 0.00E+00 |
| 2:1:10 | 7.50E−19 | 0.00E+00 | 7.20E−35 |

TABLE 4
The p values associated with in vivo HDR percentage as determined
by TIDER analysis of DNA sanger sequencing are reported above for
each of the replicates (N = 4 in all cases except for 1:1:3 where N = 3).

| Cas9 | | | | |
|---|---|---|---|---|
| mRNA:sgRNA:ssDNA | N = 1 | N = 2 | N = 3 | N = 4 |
| 1:1:8 | 0.012 | 0.009 | 0.043 | 0.01 |
| 1:1:3 | 5.70E−15 | 4.40E−09 | 1.50E−08 | |
| 2:1:3 | 3.30E−12 | 9.60E−14 | 3.20E−25 | 3.80E−19 |

In summary, a one-pot, non-viral delivery platform capable of inducing HDR-mediated correction of a single amino acid mutation in vivo has been developed. These results improve the fundamental understanding of non-viral gene editing with respect to the components necessary for achieving HDR and lay the foundation for accomplishing gene editing and correction in numerous genetic diseases that are present in cells and tissues with a high turnover rate. With further advancement, non-viral all-in-one nanoparticles may advance treatment options for those with many crippling genetic diseases and cancer.

All of the compositions and methods disclosed and claimed herein can be made and executed without undue experimentation in light of the present disclosure. While the compositions and methods of this invention have been described in terms of certain embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A composition comprising a lipid composition (e.g., nanoparticle) assembled with a gene or transcript editing composition, wherein:
(A) the gene or transcript editing composition comprises one or more of the following nucleic acids (1)-(3):
(1) a polynucleotide comprising a sequence encoding a polynucleotide-guided nuclease;
(2) a guide polynucleotide (e.g., configured to complex with at least a portion of a target gene or transcript, or a polynucleotide comprising a sequence that encodes the guide polynucleotide); and
(3) a donor polynucleotide (e.g., configured to repair a modified target gene or transcript), and
(B) the lipid composition comprises at least one ionizable lipid.

2. The composition according to claim 1, wherein the gene or transcript editing composition comprises two or more of the nucleic acids (1)-(3).

3. The composition according to claim 1, wherein the gene or transcript editing composition comprises at least the nucleic acids (1) and (2).

4. The composition according to claim 1, wherein the gene or transcript editing composition comprises all three of the nucleic acids (1)-(3).

5. The composition according to any one of claims 1-4, wherein, when assembled with the lipid composition, the one or more of the nucleic acids (1)-(3) are each encapsulated within the lipid composition.

6. The composition according to any one of claims 1-5, wherein the composition is capable of modifying the target gene or transcript in a cell.

7. The composition according to any one of claims 1-6, wherein the composition is capable of correcting an error (in the target gene) in the genome of a cell.

8. The composition according to claim 7, wherein the genome of the cell does not encode for the wild type gene.

9. The composition according to any one of claims 6-8, wherein the cell exhibits or is determined to exhibit an aberrant expression or activity of the target gene or transcript.

10. The composition according to any one of claims 1-9, wherein the polynucleotide-guided nuclease is a clustered regularly interspaced short palindromic repeats (CRISPR)-associated (Cas) nuclease.

11. The composition according to any one of claims 1-10, wherein the polynucleotide of (1) comprises from about 250 nucleotides to about 15,000 nucleotides.

12. The composition according to any one of claims 1-10, wherein the polynucleotide of (1) comprises from about 500 nucleotides to about 5,000 nucleotides.

13. The composition according to any one of claims 1-10, wherein the polynucleotide of (1) comprises from about 800 nucleotides to about 2,500 nucleotides.

14. The composition according to any one of claims 1-13, wherein the polynucleotide of (1) is a messenger ribonucleic acid (mRNA).

15. The composition according to claim 14, wherein the mRNA encodes a Cas (e.g., Cas9) protein.

16. The composition according to any one of claims 1-15, wherein the guide polynucleotide of (2) is a ribonucleic acid (RNA) (e.g., single guide RNA).

17. The composition according to any one of claims 1-16, wherein the guide polynucleotide of (2) comprises from about 25 nucleotides to about 500 nucleotides.

18. The composition according to any one of claims 1-16, wherein the guide polynucleotide of (2) comprises from about 50 nucleotides to about 300 nucleotides.

19. The composition according to any one of claims 1-16, wherein the guide polynucleotide of (2) comprises from about 80 nucleotides to about 200 nucleotides.

20. The composition according to any one of claims 1-19, wherein the donor polynucleotide of (3) is a deoxyribonucleic acid (DNA) (e.g., a single stranded DNA).

21. The composition according to any one of claims 1-20, wherein the donor polynucleotide of (3) comprises from about 25 nucleotides to about 2,500 nucleotides.

22. The composition according to any one of claims 1-20, wherein the donor polynucleotide of (3) comprises from about 25 nucleotides to about 500 nucleotides.

23. The composition according to any one of claims 1-20, wherein the donor polynucleotide of (3) comprises from about 50 nucleotides to about 300 nucleotides.

24. The composition according to any one of claims 1-20, wherein the donor polynucleotide of (3) comprises from about 80 nucleotides to about 200 nucleotides.

25. The composition according to any one of claims 1-24, wherein the donor polynucleotide of (3) comprises a donor template sequence having no more than 5, 4, 3, or 2 mismatches to at least a portion of the target gene or transcript (e.g., a portion of the genome of a cell).

26. The composition according to any one of claims 1-24, wherein the donor polynucleotide of (3) comprises a donor template sequence having no more than 3 or 2 mismatches to at least a portion of the target gene or transcript (e.g., a portion of the genome of a cell).

27. The composition according to any one of claims 1-26, wherein the DNA contains a modification relative to the genome of a cell.

28. The composition according to any one of claims 1-27, wherein the donor polynucleotide of (3) comprises a donor template sequence that is at least 80%, 81%, 82%, 83%, 84%, 85%, 86%, 87%, 88%, 89%, 90%, 91%, 92%, 93%, 94%, 95%, 96%, 97%, 98%, 99% complementary or identical to at least a portion of the target gene or transcript (e.g., a portion of the genome of a cell).

29. The composition according to any one of claims 1-28, wherein the donor polynucleotide of (3) comprises a donor template sequence that is complementary or identical to at least a portion of the target gene or transcript (e.g., a portion of the genome of a cell).

30. The composition according to claim 29, wherein the donor polynucleotide encodes for the wild type gene.

31. The composition according to any one of claims 1-30, wherein the donor polynucleotide of (3) is configured to correct an error in the genome of a cell.

32. The composition according to any one of claims 1-31, wherein the composition comprises a weight ratio of the nuclease-encoding polynucleotide (e.g., mRNA) to the guide polynucleotide from about 10:1 to about 1:5.

33. The composition according to claim 32, wherein the weight ratio of the nuclease-encoding polynucleotide (e.g., mRNA) to the guide polynucleotide is from about 5:1 to about 1:3.

34. The composition according to claim 33, wherein the weight ratio of the nuclease-encoding polynucleotide (e.g., mRNA) to the guide polynucleotide is from about 3:1 to about 1:2.

35. The composition according to claim 34, wherein the weight ratio of the nuclease-encoding polynucleotide (e.g., mRNA) to the guide polynucleotide is 2:1, 1:1, or 1:2.

36. The composition according to any one of claims 1-35, wherein the composition comprises a weight ratio of the nuclease-encoding polynucleotide (e.g., mRNA) to the donor polynucleotide from about 2:1 to about 1:20.

37. The composition according to claim 36, wherein the weight ratio of the nuclease-encoding polynucleotide (e.g., mRNA) to the donor polynucleotide is from about 1:1 to about 1:10.

38. The composition according to claim 37, wherein the weight ratio of the nuclease-encoding polynucleotide (e.g., mRNA) to the donor polynucleotide is from about 1:2 to about 1:8.

39. The composition according to claim 38, wherein the weight ratio of the nuclease-encoding polynucleotide (e.g., mRNA) to the donor polynucleotide is 1:3 or 1:4.

40. The composition according to any one of claims 1-39, wherein the composition comprises a weight ratio of the guide polynucleotide to the donor polynucleotide from about 4:1 to about 1:10.

41. The composition according to claim 40, wherein the weight ratio of the guide polynucleotide to the donor polynucleotide is from about 2:1 to about 1:8.

42. The composition according to claim 41, wherein the weight ratio of the guide polynucleotide to the donor polynucleotide is from about 1:1 to about 1:4.

43. The composition according to claim 42, wherein the weight ratio of the guide polynucleotide to the donor polynucleotide is 2:3 or 1:2.

44. The composition according to any one of claims 1-43, wherein the ionizable lipid is a cationic lipid.

45. The composition according to any one of claims 1-44, wherein the ionizable lipid is a dendron or dendrimer.

46. The composition according to any one of claims 1-45, wherein the ionizable lipid is a compound of the formula:
Core-Repeating Unit-Terminating Group (D-I)
wherein the core is linked to the repeating unit by removing one or more hydrogen atoms from the core and replacing the atom with the repeating unit and wherein:
the core has the formula: 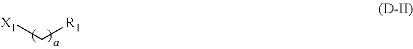
wherein:
X₁ is amino or alkylamino_{(C≤12)}, dialkylamino_{(C≤12)}, heterocycloalkyl_{(C≤12)}, heteroaryl_{(C≤12)}, or a substituted version thereof;
R₁ is amino, hydroxy, or mercapto, or alkylamino_{(C≤12)}, dialkylamino_{(C≤12)}, or a substituted version of either of these groups; and
a is 1, 2, 3, 4, 5, or 6; or
the core has the formula: 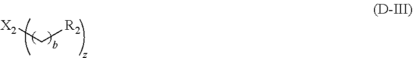
wherein:
X₂ is N(R₅)_{y};
R₅ is hydrogen, alkyl_{(C≤18)}, or substituted alkyl_{(C≤18)}; and
y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3; or
the core has the formula: 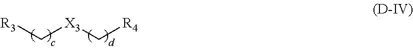
wherein:
X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d}, 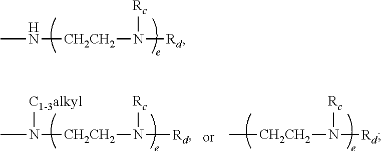
wherein:
e and f are each independently 1, 2, or 3; provided that the sum of e and f is 3;
R_{c}, R_{d}, and R_{f} are each independently hydrogen, alkyl_{(C≤6)}, or substituted alkyl_{(C≤6)};
c and d are each independently 1, 2, 3, 4, 5, or 6; or
the core is alkylamine_{(C≤18)}, dialkylamine_{(C≤36)}, heterocycloalkane_{(C≤12)}, or a substituted version of any of these groups;
wherein the repeating unit comprises a degradable diacyl and a linker;
the degradable diacyl group has the formula: 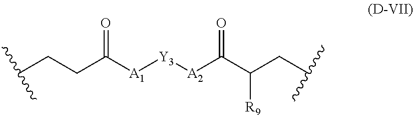
wherein:
A₁ and A₂ are each independently —O—, —S—, or —NRₐ—, wherein:
Rₐ is hydrogen, alkyl_{(C≤6)}, or substituted alkyl_{(C≤6)};
Y₃ is alkanediyl_{(C≤12)}, alkenediyl_{(C≤12)}, arenediyl_{(C≤12)}, or a substituted version of any of these groups; or a group of the formula: 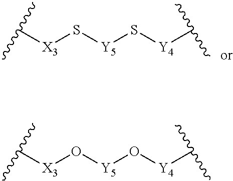
wherein:
X₃ and X₄ are alkanediyl_{(C≤12)}, alkenediyl_{(C≤12)}, arenediyl_{(C≤12)}, or a substituted version of any of these groups;
Y₅ is a covalent bond, alkanediyl_{(C≤12)}, alkenediyl_{(C≤12)}, arenediyl_{(C≤12)}, or a substituted version of any of these groups; and
R₉ is alkyl_{(C≤8)} or substituted alkyl_{(C≤8)};
the linker group has the formula: 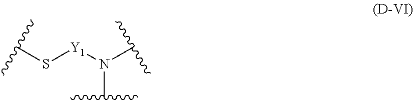
wherein:
Y₁ is alkanediyl_{(C≤12)}, alkenediyl_{(C≤12)}, arenediyl_{(C≤12)}, or a substituted version of any of these groups; and
wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
the terminating group has the formula: 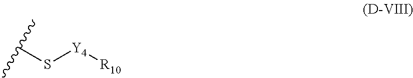
wherein:
Y₄ is alkanediyl_{(C≤24)}, alkanediyl_{(C≤24)}, or a substituted version thereof;
R₁₀ is hydrogen, amino, carboxy, hydroxy, or
aryl_{(C≤12)}, alkylamino_{(C≤12)}, dialkylamino_{(C≤12)}, N-heterocycloalkyl_{(C≤12)}, —C(O)N(R₁₁)-alkanediyl_{(C≤6)}-heterocycloalkyl_{(C≤12)}, —C(O)-alkyl-amino_{(C≤12)}, —C(O)-dialkylamino_{(C≤12)}, —C(O)—N-heterocyclo-alkyl_{(C≤12)}, wherein:
R₁₁ is hydrogen, alkyl_{(C≤6)}, or substituted alkyl_{(C≤6)};
wherein the final degradable diacyl in the chain is attached to a terminating group;
n is 0, 1, 2, 3, 4, 5, or 6;
or a pharmaceutically acceptable salt thereof.

47. The composition according to claim 46, wherein, in Formula (D-I), the core is further defined by the formula: 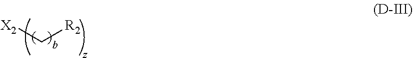
wherein:
X₂ is N(R₅)_{y};
R₅ is hydrogen or alkyl_{(C≤8)}, or substituted alkyl_{(C≤18)}; and
y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3.

48. The composition according to claim 46 or 47, wherein, in Formula (D-I), the core is further defined as: 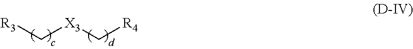
wherein:
X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d}, 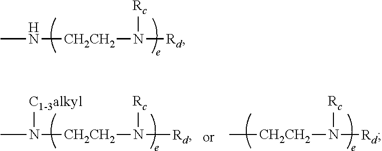
wherein:
e and f are each independently 1, 2, or 3; provided that the sum of e and f is 3;
R_{c}, R_{d}, and R_{f} are each independently hydrogen, alkyl_{(C≤6)}, or substituted alkyl_{(C≤6)};
c and d are each independently 1, 2, 3, 4, 5, or 6.

49. The composition according to any one of claims 46-48, wherein, in Formula (D-I), the core is further defined as:

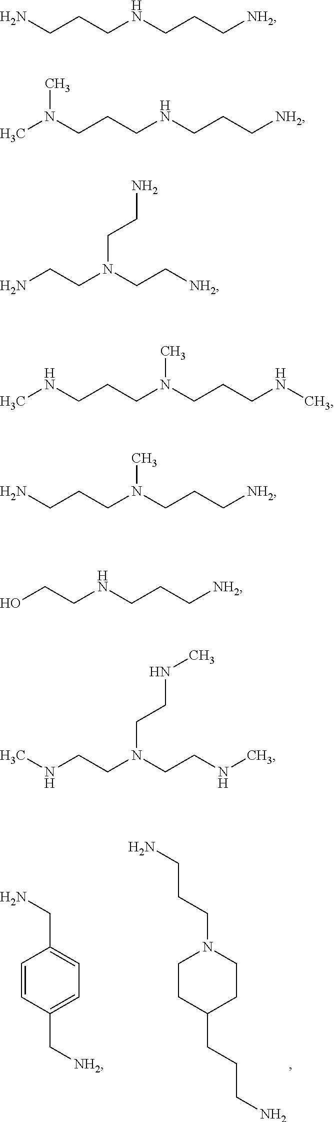

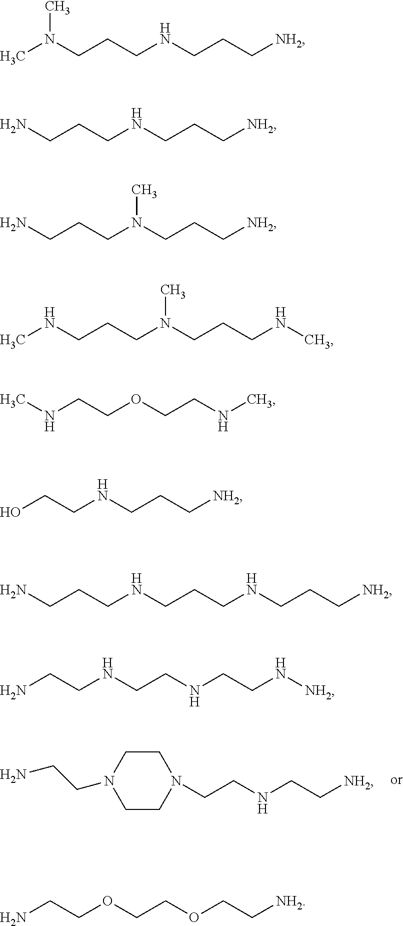

50. The composition according to any one of claims 46-49, wherein, in Formula (D-I), the core is further defined as:

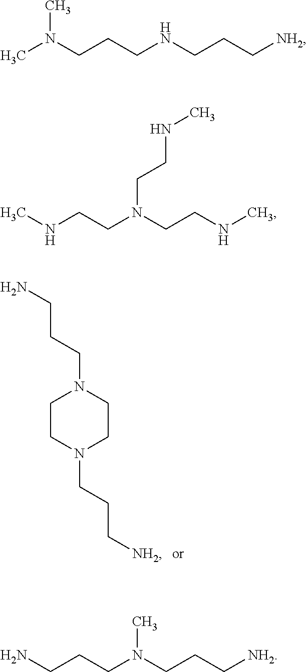

51. The composition according to any one of claims 46-50, wherein, in Formula (D-I), the core is further defined as:

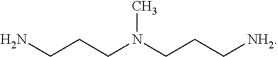

52. The composition according to any one of claims 46-51, wherein A₁ and A₂ are O.

53. The composition according to any one of claims 46-52, wherein Y₃ is alkanediyl_{(C≤12)} or substituted alkanediyl_{(C≤12)}.

54. The composition according to any one of claims 46-53, wherein Y₁ is alkanediyl_{(C≤12)} or substituted alkanediyl_{(C≤12)}.

55. The composition according to any one of claims 46-54, wherein the terminating group is further defined as: 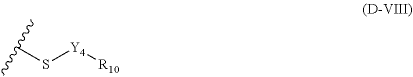
wherein:
Y₄ is alkanediyl_{(C≤18)}, or alkenediyl_{(C≤18)}; and
R₁₀ is hydrogen.

56. The composition according to any one of claims 46-55, wherein the terminating group is further defined as: 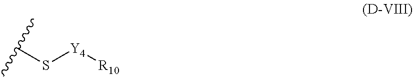
wherein:
Y₄ is alkanediyl_{(C≤18)}; and
R₁₀ is hydrogen.

57. The composition according to any one of claims 46-56, wherein the dendrimer or dendron is further defined as: 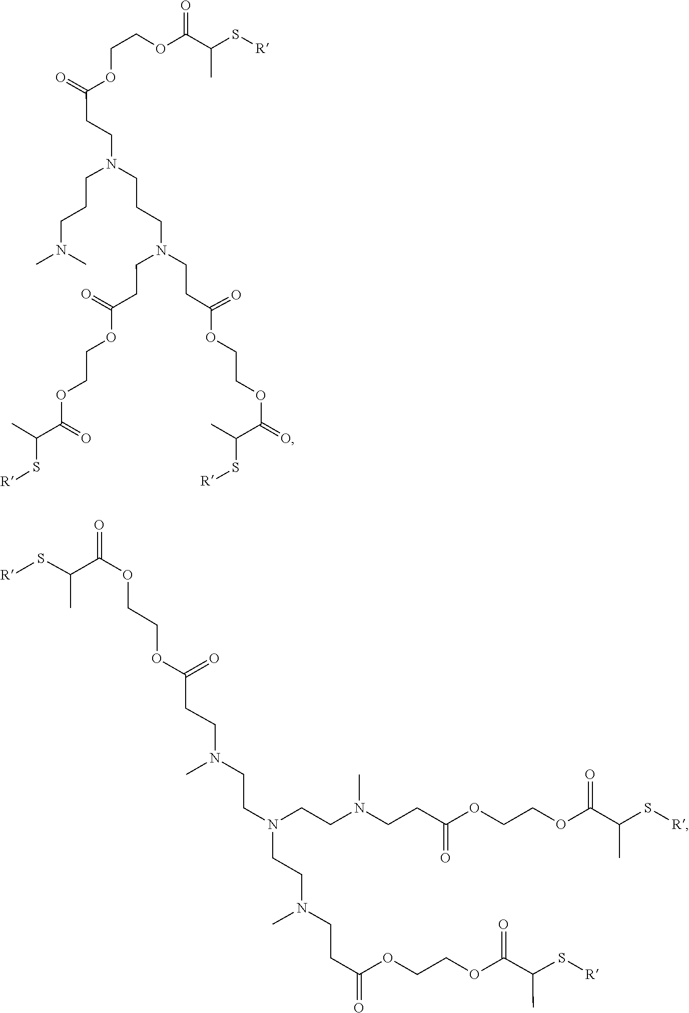 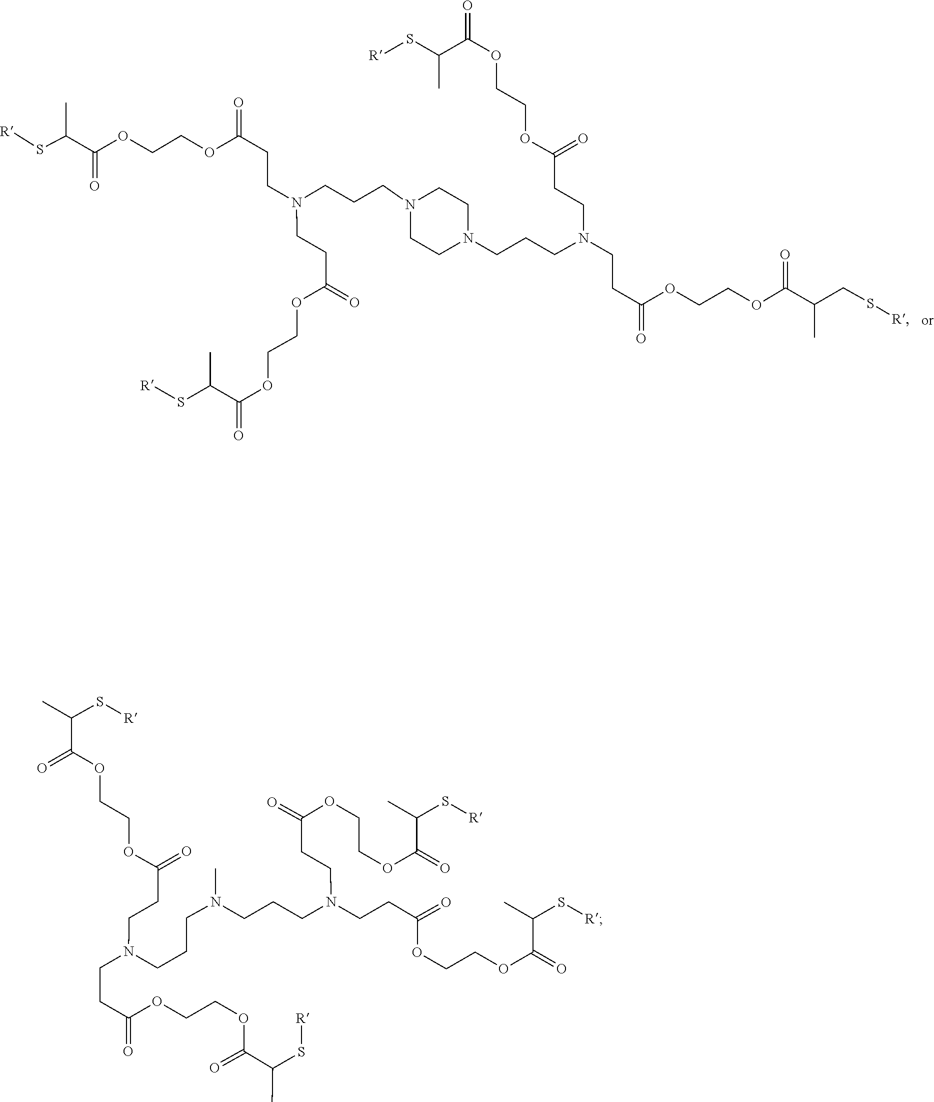
wherein:
R′ is alkyl_{(C≤18)}, alkenyl_{(C≤18)}, or a substituted version thereof.

58. The composition according to claim 46-57, wherein the dendrimer or dendron is further defined as: 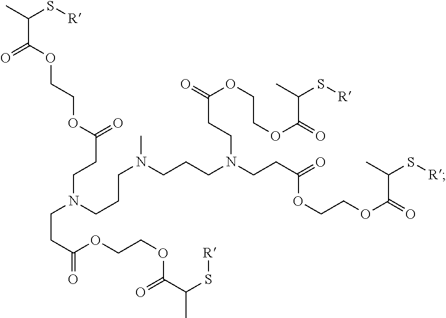
wherein:
R′ is alkyl_{(C≤18)}, alkenyl_{(C≤18)}, or a substituted version thereof.

59. The composition according to claim 58, wherein the dendrimer or dendron is further defined as: 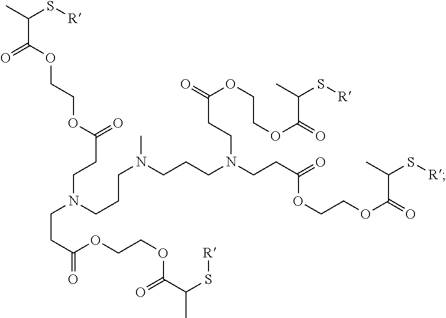
wherein:
R′ is alkyl_{(C6-18)}.

60. The composition according to any one of claims 1-45, wherein the ionizable cationic lipid is a dendrimer or dendron of a generation (g) having a structural formula: 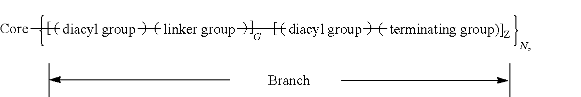
or a pharmaceutically acceptable salt thereof, wherein:
(a) the core comprises a structural formula (X_{Core}): 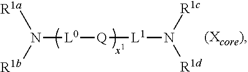
wherein:
Q is independently at each occurrence a covalent bond, —O—, —S—, —NR²—, or —CR^{3a}R^{3b}—;
R² is independently at each occurrence R^{1g} or -L²-NR^{1e}R^{1f};
R^{3a} and R^{3b} are each independently at each occurrence hydrogen or an optionally substituted (e.g., C₁-C₆, such as C₁-C₃) alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
x¹ is 0, 1, 2, 3, 4, 5, or 6; and
(b) each branch of the plurality (N) of branches independently comprises a structural formula (X_{Branch}): 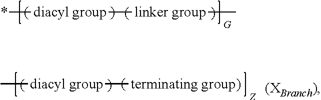
wherein:
* indicates a point of attachment of the branch to the core;
g is 1, 2, 3, or 4;
Z=2^{(g-1)};
G=0, when g=1; or G=Σᵢ₌₀^{i=g-2}2ⁱ, when g≠1;
(c) each diacyl group independently comprises a structural formula 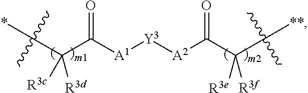
wherein:
* indicates a point of attachment of the diacyl group at the proximal end thereof;
** indicates a point of attachment of the diacyl group at the distal end thereof;
Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
A¹ and A² are each independently at each occurrence —O—, —S—, or —NR⁴—, wherein:
R⁴ is hydrogen or optionally substituted (e.g., C₁-C₆) alkyl;
m¹ and m² are each independently at each occurrence 1, 2, or 3; and
R^{3c}, R^{3d}, R^{3e} and R^{3f} are each independently at each occurrence hydrogen or an optionally substituted (e.g., C₁-C₅) alkyl; and
(d) each linker group independently comprises a structural formula 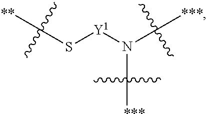
wherein:
** indicates a point of attachment of the linker to a proximal diacyl group;
*** indicates a point of attachment of the linker to a distal diacyl group; and
Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

61. The composition of claim 60, wherein x¹ is 0, 1, 2, or 3.

62. The composition of claim 60 or 61, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl 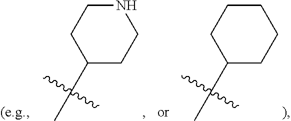 [SMILES: *C1CCNCC1 ; *C1CCCCC1]
N—(C₁-C₃ alkyl)-piperidinyl 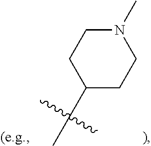 [SMILES: *C1CCN(C)CC1]
piperazinyl 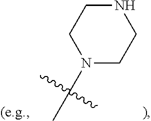 [SMILES: *N1CCNCC1]
N—(C₁-C₃ alkyl)-piperadizinyl 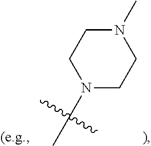 [SMILES: *N1CCN(C)CC1]
morpholinyl 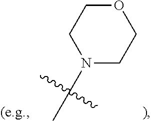 [SMILES: *N1CCOCC1]
N-pyrrolidinyl 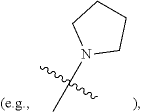 [SMILES: *N1CCCC1]
pyrrolidinyl 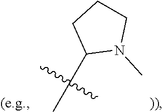 [SMILES: *C1CCCN1C]
or N—(C₁-C₃ alkyl)-pyrrolidinyl 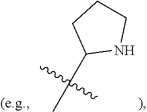 [SMILES: *C1CCCN1]
(e.g., C₆-C₁₀) aryl, and C₃-C₅ heteroaryl (e.g., imidazolyl 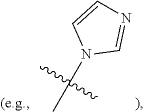 [SMILES: *n1ccnc1]
or pyridinyl

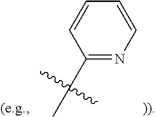 [SMILES: *c1ccccn1]

63. The composition of claim 62, wherein R₁ₐ, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

64. The composition of any one of claims 60-63, wherein R^{3a} and R^{3b} are each independently at each occurrence hydrogen.

65. The composition of any one of claims 60-64, wherein the plurality (N) of branches comprises at least 3 (e.g., at least 4, or at least 5) branches.

66. The composition of any one of claims 60-65, wherein g=1; G=0; and Z=1.

67. The composition of claim 66, wherein each branch of the plurality of branches comprises a structural formula

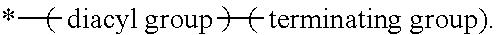

68. The composition of any one of claims 60-65, wherein g=2; G=1; and Z=2.

69. The composition of claim 68, wherein each branch of the plurality of branches comprises a structural formula

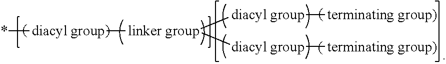

70. The composition of any one of claims 60-69, wherein the core comprises a structural formula:

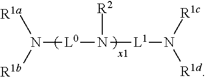

71. The composition of claim 70, wherein the core comprises a structural formula:

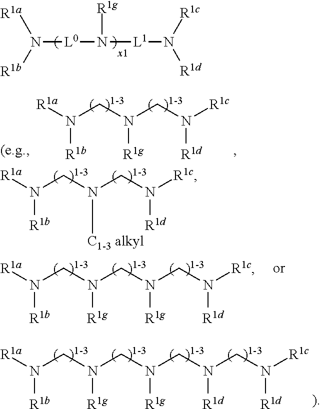

72. The composition of any one of claims 60-71, wherein the core comprises a structural formula selected from the group consisting of: 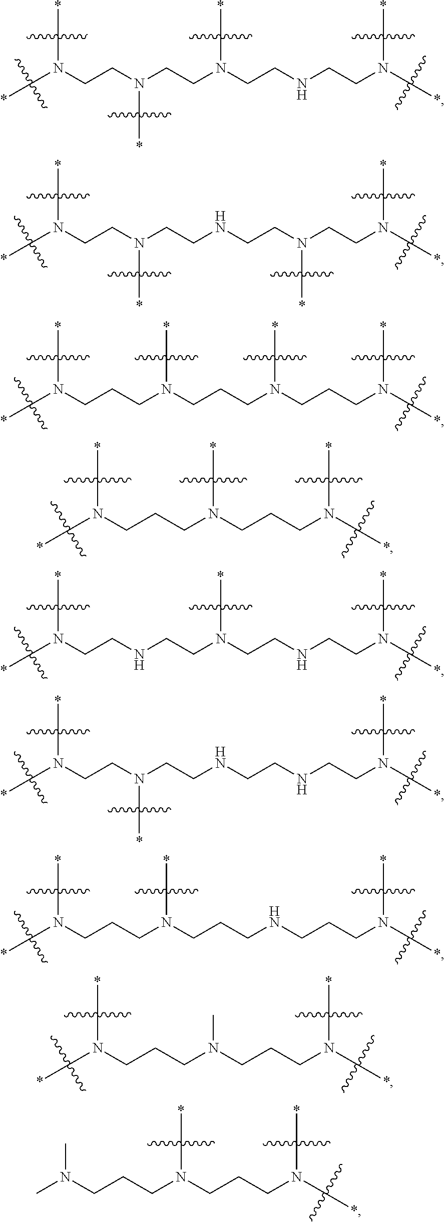
and pharmaceutically acceptable salts thereof, wherein * indicates a point of attachment of the core to a branch of the plurality of branches.

73. The composition of any one of claims 60-71, wherein the core has the structure 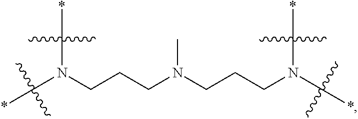
wherein * indicates a point of attachment of the core to a branch of the plurality of branches or H, wherein at least 2 (e.g., at least 3, or at least 4) branches are attached to the core.

74. The composition of any one of claims 60-71, wherein the core has the structure 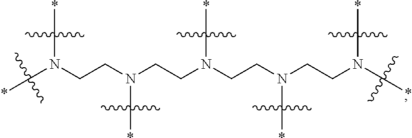
wherein * indicates a point of attachment of the core to a branch of the plurality of branches or H, wherein at least 4 (e.g., at least 5, or at least 6) branches are attached to the core.

75. The composition of any one of claims 60-74, wherein A¹ is —O— or —NH—.

76. The composition of any one of claims 60-75, wherein A² is —O— or —NH—.

77. The composition of any one of claims 60-76, wherein Y³ is C₁-C₁₂ (e.g., C₁-C₆, such as C₁-C₃) alkylene.

78. The composition of any one of claims 60-77, wherein the diacyl group independently at each occurrence comprises a structural formula 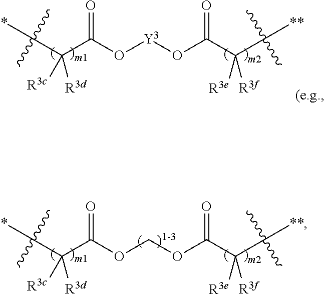
such as 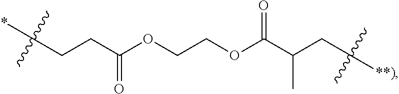
optionally wherein R^{3c}, R^{3d}, R^{3e}, and R^{3f} are each independently at each occurrence hydrogen or C₁-C₃ alkyl.

79. The composition of any one of claims 60-78, wherein each terminating group is independently C₁-C₁₈ (e.g., C₄-C₁₈) alkenylthiol or C₁-C₁₈ (e.g., C₄-C₁₈) alkylthiol.

80. The composition of any one of claims 60-79, wherein each terminating group is independently C₁-C₁₈ (e.g., C₄-C₁₈) alkenylthiol or C₁-C₁₈ (e.g., C₄-C₁₈) alkylthiol.

81. The composition according to any one of claims 1-80, wherein the composition comprises a molar ratio from about 15 to about 60 of the ionizable lipid relative to the total lipid composition.

82. The composition according to claim 81, wherein the molar ratio is from about 25 to about 50 of the ionizable lipid relative to the total lipid composition.

83. The composition according to claim 82, wherein the molar ratio is from about 30 to about 45 of the ionizable lipid relative to the total lipid composition.

84. The composition according to any one of claims 1-83, wherein the composition further comprises a phospholipid.

85. The composition according to claim 84, wherein the phospholipid comprises one or two long chain alkyl or alkenyl groups, a glycerol or a sphingosine, one or two phosphate groups, and a small organic molecule, wherein the small organic molecule is an amino acid, a sugar, or an amino substituted alkoxy group.

86. The composition according to claim 84 or 85, wherein the phospholipid is 1,2-distearoyl-sn-glycero-3-phosphocholine (DSPC) or 1,2-dioleoyl-sn-glycero-3-phosphoethanolamine (DOPE).

87. The composition according to any one of claims 84-86, wherein the phospholipid is DOPE.

88. The composition according to any one of claims 1-87, wherein the composition comprises a molar ratio from about 5 to about 50 of the phospholipid relative to the total lipid composition.

89. The composition according to claim 88, wherein the molar ratio is from about 10 to about 45 of the phospholipid relative to the total lipid composition.

90. The composition according to claim 89, wherein the molar ratio is from about 20 to about 40 of the phospholipid relative to the total lipid composition.

91. The composition according to any one of claims 1-90, wherein the composition further comprises a steroid.

92. The composition according to claim 91, wherein the steroid is cholesterol.

93. The composition according to claim 91 or 92, wherein the composition comprises a molar ratio from about 10 to about 60 of the steroid relative to the total lipid composition.

94. The composition according to claim 93, wherein the molar ratio is from about 15 to about 50 of the steroid relative to the total lipid composition.

95. The composition according to claim 94, wherein the molar ratio is from about 25 to about 50 of the steroid relative to the total lipid composition.

96. The composition according to any one of claims 1-95, wherein the composition further comprises a polymer-conjugated (e.g., PEGylated) lipid.

97. The composition according to claim 96, wherein the polymer-conjugated lipid comprises a polyethylene glycol (PEG) component from about 1000 to about 10,000 daltons.

98. The composition according to claim 96 or 97, wherein the polymer-conjugated lipid is a PEGylated diacylglycerol.

99. The composition according to claim 98, wherein the polymer-conjugated lipid is further defined by the formula: 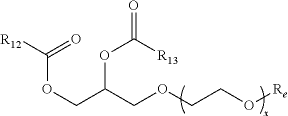
wherein:
R₁₂ and R₁₃ are each independently alkyl_{(C≤24)}, alkenyl_{(C≤24)}, or a substituted version of either of these groups;
Rₑ is hydrogen, alkyl_{(C≤8)}, or substituted alkyl_{(C≤8)}; and
x is 1-250.

100. The composition according to any one of claims 1-97, wherein the polymer-conjugated lipid is a PEGylated dimyristoyl-sn-glycerol or a compound of the formula: 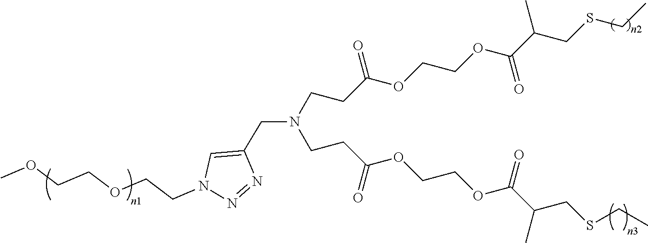
wherein:
n₁ is 5-250; and
n₂ and n₃ are each independently 2-25.

101. The composition according to any one of claims 1-100, wherein the composition comprises a molar ratio from about 0.25 to about 12.5 of the polymer-conjugated lipid relative to the total lipid composition.

102. The composition according to claim 101, wherein the molar ratio is from about 0.5 to about 10 of the polymer-conjugated lipid relative to the total lipid composition.

103. The composition according to claim 102, wherein the molar ratio is from about 1 to about 6 of the polymer-conjugated lipid relative to the total lipid composition.

104. The composition according to any one of claims 1-103, wherein the composition comprises a molar ratio of lipid components to nucleic acid components of from about 1,000:1 to about 5,000:1.

105. The composition according to claim 104, wherein the composition comprises a molar ratio of lipid components to nucleic acid components of from about 2,000:1 to about 4,000:1.

106. The composition according to claim 105, wherein the composition comprises a molar ratio of lipid components to nucleic acid components of about 2,500:1.

107. The composition according to any one of claims 1-106, wherein the composition comprises 4AC3-SC8, cholesterol, DOPE, and DMG-PEG2000.

108. The composition according to claim 107, wherein the composition comprises a molar ratio of 4AC3-SC8:cholesterol:DOPE:DMG-PEG2000 of from about 38.5:30:30:1.5.

109. The composition according to any one of claims 1-108, wherein the composition comprises an N:P ratio of from about 1:1 to about 20:1.

110. The composition according to claim 109, wherein the N:P ratio is from about 2:1 to about 10:1.

111. The composition according to claim 110, wherein the N:P ratio is from about 4:1 to about 8:1.

112. A pharmaceutical composition comprising:
(A) a composition according to any one of claims 1-111; and
(B) a pharmaceutically acceptable carrier.

113. The pharmaceutical composition of claim 112, wherein the pharmaceutical composition is formulated as a unit dose.

114. The pharmaceutical composition of claim 112 or 113, wherein the pharmaceutical composition is formulated for systemic administration.

115. The pharmaceutical composition of claim 112 or 113, wherein the pharmaceutical composition is formulated for local (e.g., injection).

116. The pharmaceutical composition of claim 112 or 113 formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

117. A composition according to any one of claims 1-116 for use in homology directed repair.

118. The composition for use according to claim 117, wherein the use comprises contacting a cell with an effective amount of a composition according to any one of claims 1-116.

119. The composition for use according to claim 117, wherein the use comprises contacting a plurality of cells with an effective amount of a composition according to any one of claims 1-116.

120. The composition for use according to claim 119, wherein said contacting provides a modified gene or transcript profile in at least 10% of the plurality of cells.

121. The composition for use according to any one of claims 118-120, wherein said contacting is in vitro.

122. The composition for use according to any one of claims 118-120, wherein said contacting is ex vivo.

123. The composition for use according to any one of claims 118-120, wherein said contacting is in vivo.

124. The composition for use according to any one of claims 118-120, wherein said contacting comprises administering the effective amount of the composition to a subject that comprises the cell or the plurality of cells.

125. The composition for use according to any one of claims 118-124, wherein said contacting provides a homology directed repair (HDR) rate of at least 1%, 5%, 10%, 15%, 20%, 25%, 30%, or 35% in the cell or the plurality of cells.

126. The composition for use according to any one of claims 118-124, wherein said contacting provides a homology directed repair (HDR) rate of at least 20%, 25%, 30%, or 35% in the cell or the plurality of cells.

127. The composition for use according to any one of claims 118-125, wherein said contacting provides an off-target or/and deletion (indel) rate of no more than 10%, 5%, 2%, or 1% in the cell or the plurality of cells.

128. The composition for use according to any one of claims 118-125, wherein said contacting provides an on-target repair rate of at least 15%, 20%, 25%, 30%, 35%, 40%, 45%, or 50% in the cell or the plurality of cells.

129. The composition for use according to any one of claims 118-128, wherein said contacting provides an on-target repair rate of at least 30%, 35%, 40%, 45%, or 50% in the cell or the plurality of cells.

130. A method for repairing a target gene or transcript in a cell, the method comprising contacting the cell with an effective amount of a composition according to any one of claims 1-129.

131. A method of performing homology directed repair (HDR) on the genome of a cell, the method comprising contacting the cell with an effective amount of a composition according to any one of claims 1-129.

132. The method of claim 130 or 131, wherein said contacting comprises contacting a plurality of cells that comprises the cell.

133. The method of any one of claims 130-132, wherein said contacting comprises administering the composition to a subject that comprises the cell.

134. A method of treating a disease or disorder in a subject in need thereof, the method comprising administering to the subject an effective amount of a composition according to any one of claims 1-129.

135. The method of claim 134, wherein the disease or disorder is a genetic disease or disorder.

136. The method of claim 134, wherein the disease or disorder is associated with an aberrant expression or activity of a target gene or transcript in the subject.

137. The method according to any one of claims 134-136, wherein the method further comprises administering a second therapy to the subject.

138. The method according to any one of claims 134-137, wherein the method further comprises administering the composition to the subject once.

139. The method according to any one of claims 134-137, wherein the method further comprises administering the composition to the subject two or more times.


SEQUENCE LISTING (13 sequences)

SEQ ID NO: 1 - DNA, 20 bp, Artificial sequence
  Synthetic nucleic acid
gctgaagcactgcacgccat

SEQ ID NO: 2 - DNA, 20 bp, Artificial sequence
  Synthetic nucleic acid
cagcatcttatctgagtgga

SEQ ID NO: 3 - DNA, 20 bp, Artificial sequence
  Synthetic nucleic acid
atggcgtgcagtgcttcagc

SEQ ID NO: 4 - DNA, 60 bp, Artificial sequence
  Synthetic primer
taatacgactcactataggggctgaagcactgcacgccatgttttagagctagaaatagc

SEQ ID NO: 5 - DNA, 4245 bp, Artificial sequence
  Synthetic nucleic acid
auggcccccaagaagaagcggaaggugggcauccacggcgugcccgccgccgacaagaaguacagcaucggccuggacaucggcaccaacagcgugggcugggccgugaucaccgacgaguacaaggugcccagcaagaaguucaaggugcugggcaacaccgaccggcacagcaucaagaagaaccugaucggcgcccugcuguucgacagcggcgagaccgccgaggccacccggcugaagcggaccgcccggcggcgguacacccggcggaagaaccggaucugcuaccugcaggagaucuucagcaacgagauggccaagguggacgacagcuucuuccaccggcuggaggagagcuuccugguggaggaggacaagaagcacgagcggcaccccaucuucggcaacaucguggacgagguggccuaccacgagaaguaccccaccaucuaccaccugcggaagaagcugguggacagcaccgacaaggccgaccugcggcugaucuaccuggcccuggcccacaugaucaaguuccggggccacuuccugaucgagggcgaccugaaccccgacaacagcgacguggacaagcuguucauccagcuggugcagaccuacaaccagcuguucgaggagaaccccaucaacgccagcggcguggacgccaaggccauccugagcgcccggcugagcaagagccggcggcuggagaaccugaucgcccagcugcccggcgagaagaagaacggccuguucggcaaccugaucgcccugagccugggccugacccccaacuucaagagcaacuucgaccuggccgaggacgccaagcugcagcugagcaaggacaccuacgacgacgaccuggacaaccugcuggcccagaucggcgaccaguacgccgaccuguuccuggccgccaagaaccugagcgacgccauccugcugagcgacauccugcgggugaacaccgagaucaccaaggccccccugagcgccagcaugaucaagcgguacgacgagcaccaccaggaccugacccugcugaaggcccuggugcggcagcagcugcccgagaaguacaaggagaucuucuucgaccagagcaagaacggcuacgccggcuacaucgacggcggcgccagccaggaggaguucuacaaguucaucaagcccauccuggagaagauggacggcaccgaggagcugcuggugaagcugaaccgggaggaccugcugcggaagcagcggaccuucgacaacggcagcaucccccaccagauccaccugggcgagcugcacgccauccugcggcggcaggaggacuucuaccccuuccugaaggacaaccgggagaagaucgagaagauccugaccuuccggauccccuacuacgugggcccccuggcccggggcaacagccgguucgccuggaugacccggaagagcgaggagaccaucacccccuggaacuucgaggaggugguggacaagggcgccagcgcccagagcuucaucgagcggaugaccaacuucgacaagaaccugcccaacgagaaggugcugcccaagcacagccugcuguacgaguacuucaccguguacaacgagcugaccaaggugaaguacgugaccgagggcaugcggaagcccgccuuccugagcggcgagcagaagaaggccaucguggaccugcuguucaagaccaaccggaaggugaccgugaagcagcugaaggaggacuacuucaagaagaucgagugcuucgacagcguggagaucagcggcguggaggaccgguucaacgccagccugggcaccuaccacgaccugcugaagaucaucaaggacaaggacuuccuggacaacgaggagaacgaggacauccuggaggacaucgugcugacccugacccuguucgaggaccgggagaugaucgaggagcggcugaagaccuacgcccaccuguucgacgacaaggugaugaagcagcugaagcggcggcgguacaccggcuggggccggcugagccggaagcugaucaacggcauccgggacaagcagagcggcaagaccauccuggacuuccugaagagcgacggcuucgccaaccggaacuucaugcagcugauccacgacgacagccugaccuucaaggaggacauccagaaggcccaggugagcggccagggcgacagccugcacgagcacaucgccaaccuggccggcagccccgccaucaagaagggcauccugcagaccgugaaggugguggacgagcuggugaaggugaugggccggcacaagcccgagaacaucgugaucgagauggcccgggagaaccagaccacccagaagggccagaagaacagccgggagcggaugaagcggaucgaggagggcaucaaggagcugggcagccagauccugaaggagcaccccguggagaacacccagcugcagaacgagaagcuguaccuguacuaccugcagaacggccgggacauguacguggaccaggagcuggacaucaaccggcugagcgacuacgacguggaccacaucgugccccagagcuuccugaaggacgacagcaucgacaacaaggugcugacccggagcgacaagaaccggggcaagagcgacaacgugcccagcgaggagguggugaagaagaugaagaacuacuggcggcagcugcugaacgccaagcugaucacccagcggaaguucgacaaccugaccaaggccgagcggggcggccugagcgagcuggacaaggccggcuucaucaagcggcagcugguggagacccggcagaucaccaagcacguggcccagauccuggacagccggaugaacaccaaguacgacgagaacgacaagcugauccgggaggugaaggugaucacccugaagagcaagcuggugagcgacuuccggaaggacuuccaguucuacaaggugcgggagaucaacaacuaccaccacgcccacgacgccuaccugaacgccguggugggcaccgcccugaucaagaaguaccccaagcuggagagcgaguucguguacggcgacuacaagguguacgacgugcggaagaugaucgccaagagcgagcaggagaucggcaaggccaccgccaaguacuucuucuacagcaacaucaugaacuucuucaagaccgagaucacccuggccaacggcgagauccggaagcggccccugaucgagaccaacggcgagaccggcgagaucgugugggacaagggccgggacuucgccaccgugcggaaggugcugagcaugccccaggugaacaucgugaagaagaccgaggugcagaccggcggcuucagcaaggagagcauccugcccaagcggaacagcgacaagcugaucgcccggaagaaggacugggaccccaagaaguacggcggcuucgacagccccaccguggccuacagcgugcuggugguggccaagguggagaagggcaagagcaagaagcugaagagcgugaaggagcugcugggcaucaccaucauggagcggagcagcuucgagaagaaccccaucgacuuccuggaggccaagggcuacaaggaggugaagaaggaccugaucaucaagcugcccaaguacagccuguucgagcuggagaacggccggaagcggaugcuggccagcgccggcgagcugcagaagggcaacgagcuggcccugcccagcaaguacgugaacuuccuguaccuggccagccacuacgagaagcugaagggcagccccgaggacaacgagcagaagcagcuguucguggagcagcacaagcacuaccuggacgagaucaucgagcagaucagcgaguucagcaagcgggugauccuggccgacgccaaccuggacaaggugcugagcgccuacaacaagcaccgggacaagcccauccgggagcaggccgagaacaucauccaccuguucacccugaccaaccugggcgcccccgccgccuucaaguacuucgacaccaccaucgaccggaagcgguacaccagcaccaaggaggugcuggacgccacccugauccaccagagcaucaccggccuguacgagacccggaucgaccugagccagcugggcggcgacagcggcggcaagcggcccgccgccaccaagaaggccggccaggccaagaagaagaagggcagcuaccccuacgacgugcccgacuacgccuga

SEQ ID NO: 6 - DNA, 120 bp, Artificial sequence
  Synthetic nucleic acid
ccaggcctctgattcctcactgattgctcttaggtctggcccctcctcagcatcttatccgcgttgaaggaaatttgcgtgtggagtatttggatgacagaaacacttttcggcatagtg

SEQ ID NO: 7 - DNA, 127 bp, Artificial sequence
  Synthetic nucleic acid
gccacctacggcaagctgaccctgaagttcatctgcaccaccggcaagctgcccgtgccctggcccaccctcgtgaccaccctgacgtacggcgtgcagtgcttcagccgctaccccgaccacatga

SEQ ID NO: 8 - DNA, 20 bp, Artificial sequence
  Synthetic primer
agctggctaggtaagcttgg

SEQ ID NO: 9 - DNA, 22 bp, Artificial sequence
  Synthetic primer
tgggtggagactgaagttaggc

SEQ ID NO: 10 - DNA, 20 bp, Artificial sequence
  Synthetic primer
cttgtacagctcgtccatgc

SEQ ID NO: 11 - DNA, 19 bp, Artificial sequence
  Synthetic primer
gggtgctcaggtagtggtt

SEQ ID NO: 12 - DNA, 17 bp, Artificial sequence
  Synthetic primer
ccctgacgtacggcgtg

SEQ ID NO: 13 - DNA, 17 bp, Artificial sequence
  Synthetic primer
cacgccgtacgtcaggg